(19) United States
(12) Patent Application Publication
(10) Pub. No.: US 2024/0294634 A1
(43) Pub. Date: Sep. 5, 2024

(54) TREATMENT OF CANCER WITH ILT-2 INHIBITORS

(71) Applicant: INNATE PHARMA, MARSEILLE (FR)

(72) Inventors: OLIVIER BENAC, MARSEILLE (FR); STÉPHANIE CHANTEUX, MARSEILLE (FR); IVAN PERROT, MARSEILLE (FR); BENJAMIN ROSSI, MARSEILLE (FR); NICOLAS VIAUD, AUBAGNE (FR)

(21) Appl. No.: 17/768,484

(22) PCT Filed: Oct. 13, 2020
(86) PCT No.: PCT/EP2020/078792

§ 371 Date: Apr. 13, 2022

Related U.S. Application Data
(60) Provisional application No. 62/914,595, filed on Oct. 14, 2019.

(51) Int. Cl. C07K 16/28; A61K 39/00; A61P 35/00

(52) U.S. Cl. CPC C07K 16/2803; A61P 35/00; A61K 2039/507

(57) ABSTRACT

This invention relates to the use of a NKG2A-neutralizing agent and an antibody that inhibits human ILT2 to treat cancer, particularly a head and neck squamous cell carcinoma (HNSCC), a lung cancer, optionally an NSCLC, a renal cell carcinoma, a colorectal carcinoma, a urothelial cancer or an ovarian cancer.

### CROSS-REFERENCE TO RELATED APPLICATIONS

This application claims the benefit of U.S. Provisional Application No. 62/914,595 filed 14 Oct. 2019; which is incorporated herein by reference in its entirety; including any drawings.

### REFERENCE TO SEQUENCE LISTING

The present application is being filed along with a Sequence Listing in electronic format. The Sequence Listing is provided as a file entitled “LILRB1-NKG2A_ST25”, created 12 Oct. 2020, which is 147 KB in size. The information in the electronic format of the Sequence Listing is incorporated herein by reference in its entirety.

### FIELD OF THE INVENTION

This invention relates to the use of a NKG2A-neutralizing agent and an antibody that inhibits human ILT2 to treat cancer.

### BACKGROUND OF THE INVENTION

CD94/NKG2A is an inhibitory receptor found on subsets of lymphocytes, including NK cells and CD8 T cells. CD94/NKG2A restricts cytokine release and cytotoxic responses of certain lymphocytes towards cells expressing the CD94/NKG2A-ligand HLA-E. HLA-E has also been found to be secreted in soluble form by certain tumor cells and activated endothelial cells. Antibodies that inhibit CD94/NKG2A signaling may increase the cytokine release and cytolytic activity of lymphocytes towards HLA-E positive target cells, such as responses of CD94/NKG2A-positive NK cells towards HLA-E expressing tumor cells or virally infected cells. Neutralizing anti-NKG2A antibodies may therefore be useful in the treatment of cancer.

Ig-like transcripts (ILTs), also called lymphocyte inhibitory receptors or leukocyte immunoglobulin- (Ig-) like receptors (LIR/LILRs) that correspond to CD85. This family of proteins is encoded by more than 10 genes located in the 19q13.4 chromosome, and includes both activating and inhibitory members. Inhibitory LILRs transmit signals through their long cytoplasmic tails, which contain between two and four immunoreceptor tyrosine-based inhibitory domains (ITIMs) that, upon phosphorylation, recruit SHP-1 and SHP-2 phosphatases which mediate inhibition of various intracellular signal pathways. ILT-2 is a receptor for class I MHC antigens and recognizes a broad spectrum of HLA-A, HLA-B, HLA-C and HLA-G alleles. ILT-2 (LILRB1) is also a receptor for H301/UL18, a human cytomegalovirus class I MHC homolog. Ligand binding results in inhibitory signals and down-regulation of the immune response. In addition to expression on dendritic cells (DCs), ILT2 proteins have also been reported to be expressed in NK cells.

The interactions of HLA class I molecules with ILT proteins is complex. HLA-G binds not only to ILT2 but also to ILT4 and other receptor (e.g. of the KIR family). Furthermore, many isoforms of HLA-G exist, and only the form HLA-G1 that associates with beta-2-microglobulin (and its soluble/secreted form HLA-G7) associate with bind to ILT2, whereas all forms HLA-G1, -G2, -G3, -G4, -G5, -G6 and -G7 associate with ILT4. Likewise, ILT2 and ILT4 bind not only HLA-G, but also to other MHC class I molecules. ILT2 and ILT4 use their two membrane distal domains (D1 and D2) to recognize the α3 domain and β2m subunit of MHC molecules, both of which are conserved among classical and non-classical MHC class I molecules.

NK cells are mononuclear cells that develop in the bone marrow from lymphoid progenitors, and morphological features and biological properties typically include the expression of the cluster determinants (CDs) CD16, CD56, and/or CD57; the absence of the alpha/beta or gamma/delta TCR complex on the cell surface; the ability to bind to and kill target cells that fail to express “self” major histocompatibility complex (MHC)/human leukocyte antigen (HLA) proteins; and the ability to kill tumor cells or other diseased cells that express ligands for activating NK receptors. NK cells are characterized by their ability to bind and kill several types of tumor cell lines without the need for prior immunization or activation. NK cells can also release soluble proteins and cytokines that exert a regulatory effect on the immune system; and can undergo multiple rounds of cell division and produce daughter cells with similar biologic properties as the parent cell. Normal, healthy cells are protected from lysis by NK cells. Based on their biological properties, various therapeutic strategies have been proposed in the art that rely on a modulation of NK cells. However, NK cell activity is regulated by a complex mechanism that involves both stimulating and inhibitory signals. Briefly, the lytic activity of NK cells is regulated by various cell surface receptors that transduce either positive or negative intracellular signals upon interaction with ligands on the target cell. The balance between positive and negative signals transmitted via these receptors determines whether or not a target cell is lysed (killed) by a NK cell. Several distinct NK-specific receptors have been identified that play an important role in the NK cell mediated recognition and killing of HLA Class I deficient target cells. NK cell stimulatory signals can be mediated by Natural Cytotoxicity Receptors (NCR) such as NKp30, NKp44, and NKp46; as well as NKG2C receptors, NKG2D receptors, certain activating Killer Ig-like Receptors (KIRs), and other activating NK receptors (Lanier, Annual Review of Immunology 2005; 23:225-74). Cross-linking of activating receptor proteins leads to NK cell activation resulting in increased intracellular Ca++ levels, triggering of cytotoxicity, and lymphokine release, and an activation of NK cytotoxicity against many types of target cells.

While immunotherapeutic agents have recently provided important advances in the treatment of cancer, many patients do not experience a complete and/or durable response. There remains a need for improvement immunotherapies, including improved methods of enhancing the ability of NK cells to mediate the elimination of cancer cells.

### SUMMARY OF THE INVENTION

Herein we studied neutralizing, non-depleting and non-FcγR-binding, specific anti-ILT2 antibodies that are able to induce an increase in the cytotoxic activity of primary NK cells from human donors. We observed that the combined use of neutralizing anti-NKG2A-antibodies and neutralizing anti-ILT2 antibodies resulted in yet further strongly enhanced anti-tumor activity by human NK cells. The combination was particularly effective and/or synergistic in causing NK cells to lyse cancer cells. Although the anti-ILT2 antibodies (as single agent) were able to enhance NK cell cytotoxicity towards tumor target cells bearing both HLA-E and HLA-G, the addition of NKG2A-neutralizing antibodies caused a strong potentiation of the anti-ILT2 antibodies' effect on NK cell cytotoxicity.

Provided herein is a combination treatment comprising a NKG2A-neutralizing agent (e.g. an NKG2A-neutralizing antibody) and an ILT2-neutralizing agent (e.g. an ILT2-neutralizing antibody). Such a combination treatment can be useful to relieve the inhibition of NK and CD8 T cell cytotoxicity, and/or to potentiate and/or enhance NK and CD8 T cell cytotoxicity towards tumor cells. In one embodiment, the combination treatment of the disclosure can be particularly advantageous when further combined with administration of an agent that enhances the activity of NK and/or CD8 T cells, for example an antibody that neutralizes PD-1 such as an antibody that binds PD-1 or an antibody that binds PD-L1. In one aspect, the combination can be particularly effect in the treatment of patients in combination with a PD-1-neutralizing agent in situations where a PD-1 neutralizing agent lacks or has limited anti-cancer activity, for example for treatment of an individual having low or no detectable expression of PD-L1 on tumor cells (e.g. tumor cell membrane), optionally wherein less than 10%, 5% or 1% of tumor cells express detectable PD-L1 at the cell membrane.

In another aspect, the combination treatment that comprises a neutralizing NKG2A agent and ILT2-neutralizing agent can be particularly advantageous when further combined in treatment with antibodies (e.g. that bind tumor-associated antigens) and that mediate ADCC.

In one aspect, the present invention provides methods of treating and/or preventing a cancer, methods for potentiating (or enhancing) NK and CD8 T cell cytotoxicity towards tumor cells, and/or methods for eliciting an anti-tumor immune response in an individual in need thereof, the method comprising treating the individual with an agent (e.g. an antibody) that neutralizes the inhibitory activity of NKG2A in combination with an agent (e.g. an antibody) that neutralizes the inhibitory activity of ILT-2.

In one embodiment, provided is an agent that neutralizes the inhibitory activity of NKG2A (e.g. an antibody), for use as a medicament, wherein the agent that neutralizes NKG2A is administered in combination with an agent (e.g. an antibody) that neutralizes the inhibitory activity of ILT-2. In one embodiment, the medicament is for eliciting an anti-tumor immune response in an individual in need thereof. In one embodiment, the medicament is for potentiating (or enhancing) NK and CD8 T cell cytotoxicity towards tumor cells. In one embodiment, the medicament is for increasing the activity and/or numbers of tumor-infiltrating CD8+ T cells and/or NK cells in an individual.

In one embodiment, provided is an agent that neutralizes the inhibitory activity of ILT2 (e.g. an antibody), for use in the treatment of cancer, wherein the agent that neutralizes ILT-2 is used in combination with an agent (e.g. an antibody) that neutralizes the inhibitory activity of NKG2A.

In one embodiment, provided is an agent that neutralizes the inhibitory activity of NKG2A (e.g. an antibody), for use in the treatment of cancer, wherein the agent that neutralizes NKG2A is used in combination with an agent (e.g. an antibody) that neutralizes the inhibitory activity of ILT-2.

In any aspect, the agent that neutralizes the inhibitory activity of ILT-2 and the agent that neutralizes the inhibitory activity of NKG2A are used to treat an individual in further combination with an agent that neutralizes the inhibitory activity of PD-1, e.g., an anti-PD-1 or anti-PDL1 antibody that inhibits the interaction between PD-1 and PDL1. In one embodiment, provided is an agent that neutralizes the inhibitory activity of ILT2 (e.g. an antibody), for use in the treatment of cancer, wherein the agent that neutralizes ILT-2 is used in combination with an agent (e.g. an antibody) that neutralizes the inhibitory activity of NKG2A and an agent (e.g. an antibody) that neutralizes the inhibitory activity of PD-1. In one embodiment, provided is an agent that neutralizes the inhibitory activity of NKG2A (e.g. an antibody), for use in the treatment of cancer, wherein the agent that neutralizes NKG2A is used in combination with an agent (e.g. an antibody) that neutralizes the inhibitory activity of ILT-2 and an agent (e.g. an antibody) that neutralizes the inhibitory activity of PD-1. In one embodiment, provided is an agent (e.g. an antibody) that neutralizes the inhibitory activity of PD-1, for use in the treatment of cancer, wherein the agent that neutralizes the inhibitory activity of PD-1 is used in combination with an agent (e.g. an antibody) that neutralizes the inhibitory activity of ILT-2 and an agent (e.g. an antibody) that neutralizes the inhibitory activity of NKG2A. In any embodiment, the individual has a cancer characterized by low or no detectable expression of PD-L1 on tumor cells (e.g. tumor cell membrane).

In any aspect, the agent that neutralizes the inhibitory activity of ILT-2 and the agent that neutralizes the inhibitory activity of NKG2A are used in further combination with an antibody that binds to an antigen present on a cell present in tumor or tumor adjacent tissue (e.g. tumor cell, immunosuppressive cell) and comprises an Fc domain or portion thereof that binds to a human CD16A polypeptide, wherein such antibody is capable of mediating ADCC toward a cell that expresses the antigen.

In one aspect, the present invention provides methods for treating and/or preventing a cancer, methods for potentiating (or enhancing) NK and CD8 T cell cytotoxicity towards tumor cells, and/or methods for eliciting an anti-tumor immune response in an individual in need thereof, wherein said individual has a tumor environment (e.g. tumor tissue, tumor adjacent tissue, tumor cells) characterized by the presence of HLA-E and/or HLA-G polypeptides, the method comprising treating an individual having a cancer with an agent (e.g. an antibody) that neutralizes the inhibitory activity of NKG2A in combination with an agent (e.g. an antibody) that neutralizes the inhibitory activity of ILT-2.

In one aspect, the present invention provides methods for treating and/or preventing a cancer, methods for potentiating (or enhancing) NK and CD8 T cell cytotoxicity towards tumor cells, and/or methods for eliciting an anti-tumor immune response in an individual in need thereof, the method comprising: (i) identifying an individual who has a cancer characterized by low or no detectable expression of PD-L1 on tumor cells (e.g. tumor cell membrane), and (ii) administering to the individual an agent that neutralizes the inhibitory receptor NKG2A, an agent (e.g. an antibody or antibody fragment) that neutralizes the inhibitory activity of ILT-2, and optionally further an agent that neutralizes the inhibitory activity of PD-1.

In one embodiment, provided is a method of increasing the activity and/or numbers of tumor-infiltrating CD8+ T cells and/or NK cells in an individual, the method comprising administering to the individual an effective amount of an agent that neutralizes the inhibitory receptor NKG2A, and an effective amount of an agent that neutralizes the inhibitory activity of ILT-2.

Among the agents (e.g., antibodies) that neutralize the inhibitory activity of ILT-2 are included, inter alia, molecules (e.g. an antibody or antibody fragment) that bind ILT-2. The agent that neutralizes ILT2 can be characterized by its ability to potentiate the activity of cytotoxic NK lymphocytes and/or CD8 T cells. The agents that neutralize ILT2 can in another aspect optionally be characterized by its ability to promote the development of an adaptive anti-tumor immune response, notably via the differentiation and/or proliferation of CD8 T cells into cytotoxic CD8 T cells.

In one embodiment, an anti-ILT2 antibody, e.g., an antibody or antibody fragment, comprises an immunoglobulin antigen binding domain, optionally hypervariable region, that specifically binds to a human ILT2 protein. The antibody neutralizes the inhibitory signaling of the ILT2 protein. In any embodiment, the antigen binding domain (or antibody or other protein that comprises such) can be specified as not binding to a human ILT1 protein. In any embodiment, the antigen binding domain (or antibody or other protein that comprises such) can be specified as not binding to a human ILT4 protein. In any embodiment, the antigen binding domain (or antibody or other protein that comprises such) can be specified as not binding to a human ILT5 protein. In any embodiment, the antigen binding domain (or antibody or other protein that comprises such) can be specified as not binding to a human ILT6 protein. In any embodiment, the antigen binding domain (or antibody or other protein that comprises such) can be specified as not binding to any one or more of (e.g., lacking binding to each of) ILT-1, ILT-3, ILT-5, ILT-6, ILT-7, ILT-8, ILT-9, ILT-10 and/or IL-T11 proteins; in one embodiment, the antigen binding domain (or antibody or other protein that comprises such) does not bind to any of the human ILT-1, -4, -5 or -6 proteins (e.g., the wild type proteins, the proteins having the amino acid sequences of SEQ ID NOS: 3, 5, 6 and 7 respectively). In any embodiment herein, any ILT protein (e.g., ILT-2) can be specified to be a protein expressed at the surface of a cell (e.g., a primary or donor cell, an NK cell, a T cell, a DC, a macrophage, a monocyte, a recombinant host cell made to express the protein). In another embodiment herein, any ILT protein (e.g., ILT-2) can be specified to be an isolated, recombinant and/or membrane-bound protein.

Optionally, an anti-ILT2 antibody can be specified as being an antibody fragment, a full-length antibody, a multi-specific or bi-specific antibody, that specifically binds to a human ILT2 polypeptide and neutralizes the inhibitory activity of the ILT2 polypeptide. Optionally, the ILT2 polypeptide is expressed at the surface of a cell, optionally an effector lymphocyte, an NK cell, a T cell, e.g., a primary NK cell, an NK cell or population of NK cells derived obtained, purified or isolated from a human individual (e.g. without further modification of the cells).

In one aspect, antibodies that specifically bind human ILT2 enhance the activity (e.g., cytotoxicity) of NK cells (e.g., primary NK cells) towards a target cell bearing at its surface a ligand (e.g., a natural ligand; an HLA class I protein) of ILT2, optionally an HLA-A protein, an HLA-B protein, an HLA-F protein, an HLA-G protein. Optionally the target cell additionally bears HLA-E protein at its surface.

In one embodiment, an antibody that neutralizes the inhibitory activity of ILT-2 is an antibody (e.g., an antibody fragment or a protein that comprises such a fragment) that specifically binds human ILT2 and that enhances and/or restores the cytotoxicity of NK cells (primary NK cells) in a standard 4-hour in vitro cytotoxicity assay in which NK cells that express ILT2 are incubated with target cells that express a ligand (e.g., a natural ligand; an HLA protein, HLA-G protein) of ILT2. In one embodiment the target cells are labeled with ⁵¹Cr prior to addition of NK cells, and then the killing (cytotoxicity) is estimated as proportional to the release of ⁵¹Cr from the cells to the medium. In one embodiment, the antibody or antibody fragment is capable of restoring cytotoxicity of NK cells that express ILT2 to at least the level observed with NK cells that do not express ILT2 (e.g., as determined according to the methods of the Examples herein). In one embodiment, the target cells are K562 cells made to express HLA-G, optionally further K562 cells made to express both HLA-G and HLA-E.

In any aspect herein, NK cells (e.g., primary NK cells) can be specified as being fresh NK cells purified from human donors, optionally incubated overnight at 37° C. before use. In any aspect herein, NK cells or primary NK cells can be specified as being ILT2 expressing, e.g., for use in assays the cells can be gated on ILT2 by flow cytometry.

In another embodiment, an antibody or antibody fragment (or a protein that comprises such a fragment) that specifically binds human ILT2 can be characterized by the ability to neutralize the inhibitory activity of the ILT2 polypeptide in a human macrophage. In one embodiment, the antibody increases macrophage-mediated ADCC. In one embodiment, the antibody increases activation or signaling in a human macrophage. In one embodiment, the antibody neutralizes the inhibitory activity of the ILT2 polypeptide in the presence of cells bearing natural ligands of ILT2 (e.g., HLA proteins).

In another aspect of any embodiment herein, the antibodies that bind ILT2 can be characterized as being capable of inhibiting (decreasing) the interactions between ILT2 and a HLA class I ligand(s) thereof, particularly a HLA-A, HLA-B, HLA-F and/or HLA-G protein. In one embodiment, the antibodies that bind ILT2 can be characterized as being capable of inhibiting (decreasing) the interactions between ILT2 and a target cell (e.g., tumor cell) that expresses an HLA ligand(s) of ILT-2, particularly a HLA-A, HLA-B, and/or HLA-G protein.

In one embodiment, the agent that neutralizes the activity of a human NKG2A polypeptide is an antibody that reduces the inhibitory activity of NKG2A by blocking binding of its ligand, HLA-E, i.e., the NKG2A-neutralizing antibody interferes with the binding of NKG2A by HLA-E. The anti-NKG2A antibody having the heavy chain variable regions of any one of SEQ ID NOS: 68-72 and a light chain variable region of SEQ ID NO: 73 is an example of such an antibody. In one embodiment, the antibody reduces the inhibitory activity of NKG2A without blocking binding of its ligand, HLA-E, i.e., the agent is a non-competitive antagonist and does not interfere with the binding of NKG2A by HLA-E. The anti-NKG2A antibody having the heavy and light chain variable regions of SEQ ID NOS: 110 and 111 respectively is an example of such an antibody.

In one embodiment, the anti-NKG2A agent is an antibody which binds with a significantly higher affinity to NKG2A than to one or more activating NKG2 receptors. For example, in one embodiment, the agent is an antibody which binds with a significantly higher affinity to NKG2A than to NKG2C. In an additional or alternative embodiment, the agent is an antibody which binds with a significantly higher affinity to NKG2A than to NKG2E. In an additional or alternative embodiment, the agent is an antibody which binds with a significantly higher affinity to NKG2A than to NKG2H. The antibody having the heavy chain variable region of any one of SEQ ID NOS: 68-72 and light chain variable region of SEQ ID NO: 73, respectively, binds NKG2A without binding to NKG2C, NKG2E or NKG2H.

In an additional or alternative embodiment, the anti-NKG2A agent competes with the antibody having the heavy chain variable region of any one of SEQ ID NOS: 68-72 and light chain variable region of SEQ ID NO: 73, and/or the antibody having the heavy and light chain variable regions of SEQ ID NOS: 110 and 111 respectively, in binding to CD94/NKG2A. The agent can be, e.g., a human or humanized anti-NKG2A antibody.

In one embodiment, the anti-NKG2A antibody is a humanized antibody having the heavy chain variable region of any one of SEQ ID NOS: 68-72 and light chain variable region of SEQ ID NO: 73. In one embodiment, the anti-NKG2A antibody is monalizumab.

These aspects are more fully described in, and additional aspects, features, and advantages will be apparent from, the description of the invention provided herein.

### BRIEF DESCRIPTION OF THE DRAWINGS

FIG. 1 shows the percent of ILT2 expressing cells in healthy individuals. Almost all B lymphocytes and monocytes express ILT2, conventional CD4 T cells and CD4 Treg cells do not express ILT2, but a significant fraction of CD8 T cells (about 25%), CD3+CD56+ lymphocytes (about 50%) and NK cells (about 30%) expressed ILT2.

FIGS. 2A to 2F shows the percent of ILT2 expressing cells in cancer patients compared to healthy individuals, showing monocytes (FIG. 2A), B cells (FIG. 2B), CD8 T cells (FIG. 2C), CD4 γδ T cells (FIG. 2D), CD16⁺ NK cells (FIG. 2E) and CD16⁻ NK cells (FIG. 2F). As can be seen, ILT2 was once again expressed on all monocytes and B cells. However on NK cells and CD8 T cell subsets, ILT2 was expressed more frequently with statistical significance on cells from three types of cancers, HNSCC, NSCLC and RCC, compared to the healthy individuals.

FIG. 3 shows % increase in lysis of K562-HLA-G/HLA-E tumor target cells by ILT2-expressing NK cell lines, in presence of antibodies, compared to isotype controls. Antibodies 12D12, 19F10a and commercial 292319 were significantly more effective than other antibodies in the ability to enhance NK cell cytotoxicity.

FIG. 4 shows ability of three exemplary anti-ILT2 antibodies to block the interactions between HLA-G or HLA-A2 expressed at the surface of cell lines and recombinant ILT2 protein was assessed by flow cytometry. 12D12, 18E1 and 26D8 each blocked the interaction of ILT2 with each of HLA-G or HLA-A2.

FIG. 5A is a representative figure showing the increase of % of total NK cells expressing CD137 mediated by anti-ILT2 antibodies using primary NK cells (from two human donors) and K562 tumor target cells made to express HLA-E and HLA-G. FIG. 5B is a representative figure showing the increase of % of ILT2-positive (left hand panel) and ILT2-negative (right hand panel) NK cells expressing CD137 mediated anti-ILT2 antibodies using NK cells from two human donors and HLA-A2-expressing B cell line. In each assay with ILT2-positive NK cells, 12D12, 18E1 and 26D8 potentiated NK cell cytotoxicity to a greater extent that antibody 292319. Each of FIGS. 5A and 5B shows the first donor on the top two panels and the second donor on the bottom two panels.

FIGS. 6A and 6B shows the ability of antibodies to enhance cytotoxicity of primary NK cells toward tumor target cells in terms of fold-increase of cytotoxicity marker CD137. FIG. 6A shows the ability of antibodies to enhance NK cell activation in presence of HLA-G-expressing target cells using primary NK cells from 5-12 different donors against HLA-G and HLA-E expressing K562 target cells. FIG. 6A shows the ability of antibodies to enhance NK cell activation in presence of HLA-G-expressing target cells using primary NK cells from 3-14 different donors against HLA-A2 expressing target B cells. In each case 12D12, 18E1 and 26D8 had greater enhancement of NK cytotoxicity.

FIG. 7 shows a representative example binding of the antibodies to a subset of the ILT2 domain fragment proteins anchored to the cell surface, as assessed by flow cytometry.

FIG. 8A shows a representative example of titration of antibodies 3H5, 12D12 and 27H5 for binding to mutant ILT2 proteins (mutants 1 and 2) anchored to cells, by flow cytometry, showing the these antibodies lost binding to mutants 2. FIG. 8B shows titration of antibodies 26D8, 18E1 and 27C10 for binding to D4 domain mutants 4-1, 4-1b, 4-2, 4-4 and 4-5 by flow cytometry. Antibodies 26D8 and 18E1 lost binding to mutants 4-1 and 4-2, and 26D8 furthermore lost binding to mutant 4-5, while antibody 18E1 had a decrease in binding (but not complete loss of binding) to mutant 4-5. In contrast, antibody 27C10 which did not potentiate the cytotoxicity of primary NK cells lost binding to mutant 4-5 but retained binding to 4-1 or 4-2.

FIG. 9A shows a model representing a portion of the ILT2 molecule that includes domain 1 (top portion, shaded in dark gray) and domain 2 (bottom, shaded in light gray). FIG. 9B shows a model representing a portion of the ILT2 molecule that includes domain 3 (top portion, shaded in dark gray) and domain 4 (bottom, shaded in light gray).

FIG. 10 shows the effect of the anti-ILT2 antibodies on activation of ILT2-positive NK cells and ILT2-negative NK cells from human urothelial cancer patients. Each of the anti-ILT2 antibodies 12D12, 18E1 and 26D8 caused a more than 2-fold increase in NK cell cytotoxicity toward target cells.

FIG. 11A shows the fold increase (compared to medium) in activation of NK cells following incubation with anti-ILT2 antibodies, anti-NKG2A antibodies, or the combination of anti-ILT2 and anti-NKG2A antibodies, and K562 tumor target cells, in two human donors. The combination of ILT2 and NKG2A blockade led to significantly higher NK cell cytotoxicity of each of the anti-ILT2 or anti-NKG2A agents alone. FIG. 11B shows results of NK cell phenotyping for LILRB1 and NKG2A expression in the two human donors. FIG. 11C shows the fold increase (compared to medium) in activation of NK cells following incubation with anti-ILT2 antibodies, anti-NKG2A antibodies, or the combination of anti-ILT2 and anti-NKG2A antibodies, and the WIL-2NS tumor target cells. The combination of anti-ILT2 and anti-NKG2A led to significantly higher NK cytotoxicity that of the NK cell cytotoxicity of each of the anti-ILT2 or anti-NKG2A agents alone. FIG. 11D shows the phenotyping of WIL-2NS and K562 tumor target cells for expression of ILT2 ligands.

FIG. 12 shows correlation of ILT2 expression levels in the tumor bed with survival in CCRCC patients. CCRCC patients were divided in 3 groups (high, mid and low ILT2 gene expression) according to the p-value of the Cox regression (each group must contain at least 10% of patients), and Survival probability curves were drawn for each of the 3 groups. Higher ILT2 correlated with lower probably of survival.

### DETAILED DESCRIPTION

As used in the specification, “a” or “an” may mean one or more. As used in the claim(s), when used in conjunction with the word “comprising”, the words “a” or “an” may mean one or more than one. As used herein “another” may mean at least a second or more.

Where “comprising” is used, this can optionally be replaced by “consisting essentially of” or by “consisting of”.

NKG2A (OMIM 161555, the entire disclosure of which is herein incorporated by reference) is a member of the NKG2 group of transcripts (Houchins, et al. (1991) J. Exp. Med. 173:1017-1020). NKG2A is encoded by 7 exons spanning 25 kb, showing some differential splicing. Together with CD94, NKG2A forms the heterodimeric inhibitory receptor CD94/NKG2A, found on the surface of subsets of NK cells, α/β T cells, γ/δ T cells, and NKT cells. Similar to inhibitory KIR receptors, it possesses an ITIM in its cytoplasmic domain. As used herein, “NKG2A” refers to any variant, derivative, or isoform of the NKG2A gene or encoded protein. Human NKG2A comprises 233 amino acids in 3 domains, with a cytoplasmic domain comprising residues 1-70, a transmembrane region comprising residues 71-93, and an extracellular region comprising residues 94-233, of the following sequence:

(SEQ ID NO: 67)
MDNQGVIYSDLNLPPNPKRQQRKPKGNKSSILATEQEITYAELNLQKAS
QDFQGNDKTYHCKDLPSAPEKLIVGILGIICLILMASVVTIVVIPSTLI
QRHNNSSLNTRTQKARHCGHCPEEWITYSNSCYYIGKERRTWEESLLAC
TSKNSSLLSIDNEEEMKFLSIISPSSWIGVFRNSSHHPWVTMNGLAFKH
EIKDSDNAELNCAVLQVNRLKSAQCGSSIIYHCKHKL.

NKG2C (OMIM 602891, the entire disclosure of which is herein incorporated by reference) and NKG2E (OMIM 602892, the entire disclosure of which is herein incorporated by reference) are two other members of the NKG2 group of transcripts (Gilenke, et al. (1998) Immunogenetics 48:163-173). The CD94/NKG2C and CD94/NKG2E receptors are activating receptors found on the surface of subsets of lymphocytes such as NK cells and T-cells.

HLA-E (OMIM 143010, the entire disclosure of which is herein incorporated by reference) is a nonclassical MHC molecule that is expressed on the cell surface and regulated by the binding of peptides, e.g. such as fragments derived from the signal sequence of other MHC class I molecules. Soluble versions of HLA-E have also been identified. In addition to its T-cell receptor binding properties, HLA-E binds subsets of natural killer (NK) cells, natural killer T-cells (NKT) and T cells (α/β and γ/δ), by binding specifically to CD94/NKG2A, CD94/NKG2B, and CD94/NKG2C (see, e.g., Braud et al. (1998) Nature 391:795-799, the entire disclosure of which is herein incorporated by reference). Surface expression of HLA-E protects target cells from lysis by CD94/NKG2A+NK, T, or NKT cell clones. As used herein, “HLA-E” refers to any variant, derivative, or isoform of the HLA-E gene or encoded protein.

In the context of the present invention, “NKG2A-”, or “CD94/NKG2A-”, “positive lymphocyte”, or “restricted lymphocyte”, refers to cells of the lymphoid lineage (e.g. NK-, NKT- and T-cells) expressing CD94/NKG2A on the cell-surface, which can be detected by e.g. flow-cytometry using antibodies that specifically recognize a combined epitope on CD94 and NKG2A or and epitope on NKG2A alone. “NKG2A positive lymphocyte” also includes immortal cell lines of lymphoid origin (e.g. NKL, NK-92).

In the context of the present invention, “reduces the inhibitory activity of NKG2A”, “neutralizes NKG2A” or “neutralizes the inhibitory activity of NKG2A” refers to a process in which CD94/NKG2A is inhibited in its capacity to negatively affect intracellular processes leading to lymphocyte responses such as cytokine release and cytotoxic responses. This can be measured for example in a NK- or T-cell based cytotoxicity assay, in which the capacity of a therapeutic compound to stimulate killing of HLA-E positive cells by CD94/NKG2A positive lymphocytes is measured. In one embodiment, an NKG2A-neutralizing antibody preparation causes at least a 10% augmentation in the cytotoxicity of a CD94/NKG2A-restricted lymphocyte, optionally at least a 40% or 50% augmentation in said lymphocyte cytotoxicity, optionally at least a 70% augmentation in said lymphocyte cytotoxicity”, optionally at least a 70% augmentation of NK cytotoxicity, and referring to the cytotoxicity assays described herewith. If an anti-NKG2A antibody reduces or blocks CD94/NKG2A interactions with HLA-E, it may increase the cytotoxicity of CD94/NKG2A-restricted lymphocytes. This can be evaluated, for example, in a standard 4-hour in vitro cytotoxicity assay using, e.g., NK cells that express CD94/NKG2A, and target cells that express HLA-E. Such NK cells do not efficiently kill targets that express HLA-E because CD94/NKG2A recognizes HLA-E, leading to initiation and propagation of inhibitory signaling that prevents lymphocyte-mediated cytolysis. Such an in vitro cytotoxicity assay can be carried out by standard methods that are well known in the art, as described for example in Coligan et al., eds., Current Protocols in Immunology, Greene Publishing Assoc. and Wiley Interscience, N.Y., (1992, 1993). Chromium release and/or other parameters to assess the ability of the antibody to stimulate lymphocytes to kill target cells such as P815, K562 cells, or appropriate tumor cells are also disclosed in Sivori et al., J. Exp. Med. 1997; 186:1129-1136; Vitale et al., J. Exp. Med. 1998; 187:2065-2072; Pessino et al. J. Exp. Med. 1998; 188:953-960; Neri et al. Clin. Diag. Lab. Immun. 2001; 8:1131-1135; Pende et al. J. Exp. Med. 1999; 190:1505-1516, the entire disclosures of each of which are herein incorporated by reference. The target cells are labeled with ⁵¹Cr prior to addition of NK cells, and then the killing is estimated as proportional to the release of ⁵¹Cr from the cells to the medium, as a result of killing. The addition of an antibody that prevents CD94/NKG2A from binding to HLA-E results in prevention of the initiation and propagation of inhibitory signaling via CD94/NKG2A. Therefore, addition of such agents results in increases in lymphocyte-mediated killing of the target cells. This step thereby identifies agents that prevent CD94/NKG2A-induced negative signaling by, e.g., blocking ligand binding. In a particular ⁵¹Cr-release cytotoxicity assay, CD94/NKG2A-expressing NK effector-cells can kill HLA-E-negative LCL 721.221 target cells, but less well HLA-E-expressing LCL 721.221-Cw3 control cells. In contrast, YTS effector-cells that lack CD94/NKG2A kill both cell-lines efficiently. Thus, NK effector cells kill less efficiently HLA-E⁺ LCL 721.221-Cw3 cells due to HLA-E-induced inhibitory signaling via CD94/NKG2A. When NK cells are pre-incubated with blocking anti-CD94/NKG2A antibodies described herewith in such a ⁵¹Cr-release cytotoxicity assay, HLA-E-expressing LCL 721.221-Cw3 cells are more efficiently killed, in an antibody-concentration-dependent fashion. The inhibitory activity (i.e. cytotoxicity enhancing potential) of an anti-NKG2A antibody can also be assessed in any of a number of other ways, e.g., by its effect on intracellular free calcium as described, e.g., in Sivori et al., J. Exp. Med. 1997; 186:1129-1136, the disclosure of which is herein incorporated by reference. Activation of NK cell cytotoxicity can be assessed for example by measuring an increase in cytokine production (e.g. IFN-γ production) or cytotoxicity markers (e.g. CD107 or CD137 mobilization). In an exemplary protocol, IFN-γ production from PBMC is assessed by cell surface and intracytoplasmic staining and analysis by flow cytometry after 4 days in culture. Briefly, Brefeldin A (Sigma Aldrich) is added at a final concentration of 5 μg/ml for the last 4 hours of culture. The cells are then incubated with anti-CD3 and anti-CD56 monoclonal antibody prior to permeabilization (IntraPrep™; Beckman Coulter) and staining with PE-anti-IFN-γ or PE-IgG1 (Pharmingen). GM-CSF and IFN-γ production from polyclonal activated NK cells are measured in supernatants using ELISA (GM-CSF: DuoSet Elisa, R&D Systems, Minneapolis, MN, IFN-γ: OptEIA set, Pharmingen).

Human ILT2 is a member of the lymphocyte inhibitory receptor or leukocyte immunoglobulin- (Ig-) like receptor (LIRJLILRs) family. ILT-2 includes 6 isoforms. Uniprot identifier number Q8NHL6, the entire disclosure of which is incorporated herein by reference, is referred to as the canonical sequence, comprises 650 amino acids, and has the following amino acid sequence (including the signal sequence of residues 1-23):

(SEQ ID NO: 1)
MTPILTVLIC LGLSLGPRTH VQAGHLPKPT LWAEPGSVIT QGSPVTLRCQ GGQETQEYRL
YREKKTALWI TRIPQELVKK GQFPIPSITW EHAGRYRCYY GSDTAGRSES SDPLELVVTG
AYIKPTLSAQ PSPVVNSGGN VILQCDSQVA FDGFSLCKEG EDEHPQCLNS QPHARGSSRA
IFSVGPVSPS RRWWYRCYAY DSNSPYEWSL PSDLLELLVL GVSKKPSLSV QPGPIVAPEE
TLTLQCGSDA GYNRFVLYKD GERDFLQLAG AQPQAGLSQA NFTLGPVSRS YGGQYRCYGA
HNLSSEWSAP SDPLDILIAG QFYDRVSLSV QPGPTVASGE NVTLLCQSQG WMQTFLLTKE
GAADDPWRLR STYQSQKYQA EFPMGPVTSA HAGTYRCYGS QSSKPYLLTH PSDPLELVVS
GPSGGPSSPT TGPTSTSGPE DQPLTPTGSD PQSGLGRHLG VVIGILVAVI LLLLLLLLLF
LILRHRRQGK HWTSTQRKAD FQHPAGAVGP EPTDRGLQWR SSPAADAQEE NLYAAVKHTQ
PEDGVEMDTR SPHDEDPQAV TYAEVKHSRP RREMASPPSP LSGEFLDTKD RQAEEDRQMD
TEAAASEAPQ DVTYAQLHSL TLRREATEPP PSQEGPSPAV PSIYATLAIH.

The ILT2 amino acid sequence without the leader sequence is shown below:

(SEQ ID NO: 2)
GHLPKPTLWA EPGSVITQGS PVTLRCQGGQ ETQEYRLYRE KKTALWITRI PQELVKK
GQFPIPSITW EHAGRYRCYY GSDTAGRSES SDPLELVVTG AYIKPTLSAQ PSPVVNSGGN
VILQCDSQVA FDGFSLCKEG EDEHPQCLNS QPHARGSSRA IFSVGPVSPS RRWWYRCYAY
DSNSPYEWSL PSDLLELLVL GVSKKPSLSV QPGPIVAPEE TLTLQCGSDA GYNRFVLYKD
GERDFLQLAG AQPQAGLSQA NFTLGPVSRS YGGQYRCYGA HNLSSEWSAP SDPLDILIAG
QFYDRVSLSV QPGPTVASGE NVTLLCQSQG WMQTFLLTKE GAADDPWRLR STYQSQKYQA
EFPMGPVTSA HAGTYRCYGS QSSKPYLLTH PSDPLELVVS GPSGGPSSPT TGPTSTSGPE
DQPLTPTGSD PQSGLGRHLG VVIGILVAVI LLLLLLLLLF LILRHRRQGK HWTSTQRKAD
FQHPAGAVGP EPTDRGLQWR SSPAADAQEE NLYAAVKHTQ PEDGVEMDTR SPHDEDPQAV
TYAEVKHSRP RREMASPPSP LSGEFLDTKD RQAEEDRQMD TEAAASEAPQ DVTYAQLHSL
TLRREATEPP PSQEGPSPAV PSIYATLAIH.

In the context of the present invention, “neutralize” or “neutralize the inhibitory activity of ILT2 refers to a process in which an ILT2 protein is inhibited in its capacity to negatively affect intracellular processes leading to immune cell responses (e.g., cytotoxic responses). For example, neutralization of ILT-2 can be measured for example in a standard NK- or T-cell based cytotoxicity assay, in which the capacity of a therapeutic compound to stimulate killing of HLA positive cells by ILT positive lymphocytes is measured. In one embodiment, an antibody preparation causes at least a 10% augmentation in the cytotoxicity of an ILT-2-restricted lymphocyte, optionally at least a 40% or 50% augmentation in lymphocyte cytotoxicity, or optionally at least a 70% augmentation in NK cytotoxicity, and referring to the cytotoxicity assays described. In one embodiment, an antibody preparation causes at least a 10% augmentation in cytokine release by a ILT-2-restricted lymphocyte, optionally at least a 40% or 50% augmentation in cytokine release, or optionally at least a 70% augmentation in cytokine release, and referring to the cytotoxicity assays described. In one embodiment, an antibody preparation causes at least a 10% augmentation in cell surface expression of a marker of cytotoxicity (e.g., CD107 and/or CD137) by a ILT-2-restricted lymphocyte, optionally at least a 40% or 50% augmentation, or optionally at least a 70% augmentation in cell surface expression of a marker of cytotoxicity (e.g., CD107 and/or CD137).

The ability of an anti-ILT2 antibody to “block” or “inhibit” the binding of an ILT2 molecule to a natural ligand thereof (e.g., an HLA molecule) means that the antibody, in an assay using soluble or cell-surface associated ILT2 and natural ligand (e.g., HLA molecule, for example HLA-A, HLA-B, HLA-F, HLA-G), can detectably reduce the binding of a ILT2 molecule to the ligand (e.g., an HLA molecule) in a dose-dependent fashion, where the ILT2 molecule detectably binds to the ligand (e.g., HLA molecule) in the absence of the antibody.

The term “agent” is used herein to denote a chemical compound, a mixture of chemical compounds, a biological macromolecule, or an extract made from biological materials. The term “therapeutic agent” refers to an agent that has biological activity.

The term “antibody-dependent cell-mediated cytotoxicity” or “ADCC” is a term well understood in the art, and refers to a cell-mediated reaction in which non-specific cytotoxic cells that express Fc receptors (FcRs) recognize bound antibody on a target cell and subsequently cause lysis of the target cell. Non-specific cytotoxic cells that mediate ADCC include natural killer (NK) cells, macrophages, monocytes, neutrophils, and eosinophils.

As used herein, “treatment” and “treating” and the like generally mean obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it such as a preventive early asymptomatic intervention; (b) inhibiting the disease, e.g., arresting its development; or relieving the disease, e.g., causing regression of the disease and/or its symptoms or conditions such as improvement or remediation of damage, for example in a subject who has been diagnosed as having the disease. Optionally, treatment may cause (e.g. may be characterized as a method of causing) a decrease in tumor burden, a decrease in the size and/or number of lesions, a decrease or delay in the progression of cancer (e.g., an increase in progression-free survival), a delay or prevention of cancer metastasis and/or an increase in survival. Optionally, treatment may cause or provide (e.g. may be characterized as a method of causing or providing) stable disease, a partial response or a complete response in a subject, e.g. according to standard criteria, optionally RECIST criteria.

It will be appreciated that when “treatment of cancer” or the like is mentioned with reference to an ILT2-neutralizing agent and a NKG2A-neutralizing agent (e.g., antibody), embodiments can include: (a) method of treatment of cancer, said method comprising the step of administering (for at least one treatment) an ILT2-neutralizing agent and a NKG2A-neutralizing agent, (preferably in a pharmaceutically acceptable carrier material) to an individual, a mammal, especially a human, in need of such treatment, in respective doses that allows for the treatment of cancer, (a therapeutically effective amount), preferably in doses (amount) as specified herein; (b) the use of an ILT2-neutralizing agent and a NKG2A-neutralizing agent for the treatment of cancer, or an ILT2-neutralizing agent and a NKG2A-neutralizing agent, for use in said treatment (especially in a human); (c) the use of an ILT2-neutralizing agent and a NKG2A-neutralizing agent for the manufacture of a pharmaceutical preparation for the treatment of cancer, a method of using an ILT2-neutralizing agent and a NKG2A-neutralizing agent for the manufacture of a kit or pharmaceutical preparation for the treatment of cancer, comprising admixing each of an ILT2-neutralizing agent and a NKG2A-neutralizing agent with a pharmaceutically acceptable carrier, or a kit or pharmaceutical preparation comprising an effective dose of an ILT2-neutralizing agent and a pharmaceutical preparation comprising an effective dose of an NKG2A-neutralizing agent that is appropriate for the treatment of cancer; or (d) any combination of a), b), and c), in accordance with the subject matter allowable for patenting in a country where this application is filed.

As used herein, the term “antigen binding domain” refers to a domain comprising a three-dimensional structure capable of immunospecifically binding to an epitope. Thus, in one embodiment, said domain can comprise a hypervariable region, optionally a VH and/or VL domain of an antibody chain, optionally at least a VH domain. In another embodiment, the binding domain may comprise at least one complementarity determining region (CDR) of an antibody chain. In another embodiment, the binding domain may comprise a polypeptide domain from a non-immunoglobulin scaffold.

The term “antibody,” as used herein, refers to polyclonal and monoclonal antibodies. Depending on the type of constant domain in the heavy chains, antibodies are assigned to one of five major classes: IgA, IgD, IgE, IgG, and IgM. Several of these are further divided into subclasses or isotypes, such as IgG1, IgG2, IgG3, IgG4, and the like. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids that is primarily responsible for antigen recognition. The terms variable light chain (V_{L}) and variable heavy chain (V_{H}) refer to these light and heavy chains respectively. The heavy-chain constant domains that correspond to the different classes of immunoglobulins are termed “alpha,” “delta,” “epsilon,” “gamma” and “mu,” respectively. The subunit structures and three-dimensional configurations of different classes of immunoglobulins are well known. IgG are the exemplary classes of antibodies employed herein because they are the most common antibodies in the physiological situation and because they are most easily made in a laboratory setting. Optionally the antibody is a monoclonal antibody. Particular examples of antibodies are humanized, chimeric, human, or otherwise-human-suitable antibodies. “Antibodies” also includes any fragment or derivative of any of the herein described antibodies.

The term “hypervariable region” when used herein refers to the amino acid residues of an antibody that are responsible for antigen binding. The hypervariable region generally comprises amino acid residues from a “complementarity-determining region” or “CDR” (e.g., residues 24-34 (L1), 50-56 (L2) and 89-97 (L3) in the light-chain variable domain and 31-35 (H1), 50-65 (H2) and 95-102 (H3) in the heavy-chain variable domain; Kabat et al. 1991) and/or those residues from a “hypervariable loop” (e.g., residues 26-32 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol 1987; 196:901-917), or a similar system for determining essential amino acids responsible for antigen binding. Typically, the numbering of amino acid residues in this region is performed by the method described in Kabat et al., supra. Phrases such as “Kabat position”, “variable domain residue numbering as in Kabat” and “according to Kabat” herein refer to this numbering system for heavy chain variable domains or light chain variable domains. Using the Kabat numbering system, the actual linear amino acid sequence of a peptide may contain fewer or additional amino acids corresponding to a shortening of, or insertion into, a FR or CDR of the variable domain. For example, a heavy chain variable domain may include a single amino acid insert (residue 52a according to Kabat) after residue 52 of CDR H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

By “framework” or “FR” residues as used herein is meant the region of an antibody variable domain exclusive of those regions defined as CDRs. Each antibody variable domain framework can be further subdivided into the contiguous regions separated by the CDRs (FR1, FR2, FR3 and FR4).

The term “specifically binds to” means that an antibody can bind preferably in a competitive binding assay to the binding partner, e.g. NKG2A for an anti-NKG2A agent or antibody, ILT-2 for an anti-ILT-2 antibody, as assessed using either recombinant forms of the proteins, epitopes therein, or native proteins present on the surface of isolated target cells. Competitive binding assays and other methods for determining specific binding are well known in the art. For example binding can be detected via radiolabels, physical methods such as mass spectrometry, or direct or indirect fluorescent labels detected using, e.g., cytofluorometric analysis (e.g. FACScan). Binding above the amount seen with a control, non-specific agent indicates that the agent binds to the target. An agent that specifically binds NKG2A may bind NKG2A alone or NKG2A as a dimer with CD94.

When an antibody is said to “compete with” a particular monoclonal antibody, it means that the antibody competes with the monoclonal antibody in a binding assay using either recombinant molecules (e.g., NKG2A, ILT-2) or surface expressed molecules (e.g., NKG2A, ILT-2). For example, if a test antibody reduces the binding of an antibody having a heavy chain variable region of any of SEQ ID NOS: 68-72 and a light chain variable region of SEQ ID NO: 73 to a NKG2A polypeptide or NKG2A-expressing cell in a binding assay, the antibody is said to “compete” respectively with such antibody. An antibody can for example be referred to as competing with a particular reference antibody for binding to the epitope on an antigen (e.g. NKG2A or ILT-2) bound by the reference antibody.

The term “affinity”, as used herein, means the strength of the binding of an antibody to an epitope. The affinity of an antibody is given by the dissociation constant Kd, defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant Kₐ is defined by 1/Kd. Methods for determining the affinity of monoclonal antibodies can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One standard method well known in the art for determining the affinity of monoclonal antibodies is the use of surface plasmon resonance (SPR) screening (such as by analysis with a BIAcore™ SPR analytical device).

Within the context herein a “determinant” designates a site of interaction or binding on a polypeptide.

The term “epitope” refers to an antigenic determinant, and is the area or region on an antigen to which an antibody binds. A protein epitope may comprise amino acid residues directly involved in the binding as well as amino acid residues which are effectively blocked by the specific antigen binding antibody or peptide, i.e., amino acid residues within the “footprint” of the antibody. It is the simplest form or smallest structural area on a complex antigen molecule that can combine with e.g., an antibody or a receptor. Epitopes can be linear or conformational/structural. The term “linear epitope” is defined as an epitope composed of amino acid residues that are contiguous on the linear sequence of amino acids (primary structure). The term “conformational or structural epitope” is defined as an epitope composed of amino acid residues that are not all contiguous and thus represent separated parts of the linear sequence of amino acids that are brought into proximity to one another by folding of the molecule (secondary, tertiary and/or quaternary structures). A conformational epitope is dependent on the 3-dimensional structure. The term ‘conformational’ is therefore often used interchangeably with ‘structural’.

The terms “Fc domain,” “Fc portion,” and “Fc region” refer to a C-terminal fragment of an antibody heavy chain, e.g., from about amino acid (aa) 230 to about aa 450 of human γ (gamma) heavy chain or its counterpart sequence in other types of antibody heavy chains (e.g., α, δ, ε and μ for human antibodies), or a naturally occurring allotype thereof. Unless otherwise specified, the commonly accepted Kabat amino acid numbering for immunoglobulins is used throughout this disclosure (see Kabat et al. (1991) Sequences of Protein of Immunological Interest, 5th ed., United States Public Health Service, National Institute of Health, Bethesda, MD).

The terms “isolated”, “purified” or “biologically pure” refer to material that is substantially or essentially free from components which normally accompany it as found in its native state. Purity and homogeneity are typically determined using analytical chemistry techniques such as polyacrylamide gel electrophoresis or high performance liquid chromatography. A protein that is the predominant species present in a preparation is substantially purified.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (nonrecombinant) form of the cell or express native genes that are otherwise abnormally expressed, under expressed or not expressed at all.

Within the context herein, the term antibody that “binds” a polypeptide or epitope designates an antibody that binds said determinant with specificity and/or affinity.

The term “identity” or “identical”, when used in a relationship between the sequences of two or more polypeptides, refers to the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

Methods for determining identity are designed to give the largest match between the sequences tested. Methods of determining identity are described in publicly available computer programs. Computer program methods for determining identity between two sequences include the GCG program package, including GAP (Devereux et al., Nucl. Acid. Res. 12, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well-known Smith Waterman algorithm may also be used to determine identity.

### NKG2A-Neutralizing Therapeutic Agents

Example of NKG2A neutralizing agents include in particular protein agents (e.g. antibodies and antibody fragments, as proteins that comprise such) that bind an extra-cellular portion of human CD94/NKG2A receptor or its ligand HLA-E and reduces the inhibitory activity of human CD94/NKG2A receptor expressed on the surface of a CD94/NKG2A positive lymphocyte. In one embodiment the agent competes with HLA-E in binding to CD94/NKG2A, i.e. the agent blocks the interaction between CD94/NKG2A and its ligand HLA-E. In another embodiment the agent binds NKG2A but does not compete with HLA-E in binding to CD94/NKG2A; i.e. the agent is capable of binding CD94/NKG2A simultaneously with HLA-E.

In one embodiment, the agent (e.g. an antibody or antibody fragment) comprises an antigen binding domain that binds to NKG2A. The antibody may bind a combined epitope on CD94 and NKG2A or and epitope on NKG2A alone. In another embodiment, the agent (e.g. an antibody or antibody fragment) comprises an antigen binding domain that binds to HLA-E and inhibits the interaction between human HLA-E and human NKG2A proteins.

In one aspect the NKG2A neutralizing agent comprises an antibody selected from a fully human antibody, a humanized antibody, and a chimeric antibody. In one aspect, the agent comprises a constant domain derived from a human IgG1, IgG2, IgG3 or IgG4 antibody. In one aspect, the agent is a fragment of an antibody selected from IgA, an IgD, an IgG, an IgE and an IgM antibody. In one aspect, the agent is an antibody fragment selected from a Fab fragment, a Fab′ fragment, a Fab′-SH fragment, a F(ab)2 fragment, a F(ab′)2 fragment, an Fv fragment, a Heavy chain Ig (a llama or camel Ig), a V_{HH} fragment, a single domain FV, and a single-chain antibody fragment. In one aspect, the agent is a synthetic or semisynthetic antibody-derived molecule selected from a scFV, a dsFV, a minibody, a diabody, a triabody, a kappa body, an IgNAR, and a multispecific antibody.

Generally, the anti-NKG2A antibodies will not demonstrate substantial specific binding (e.g., via their Fc domains) to human Fcγ receptors, e.g. CD16. Optionally, the anti-NKG2A antibodies lack substantial specific binding or have low or decreased specific binding to one or more, or all of, human CD16, CD32A, CD32B or CD64. Exemplary antibodies may comprise constant regions of various heavy chains that are known not to bind or to have low binding to Fcγ receptors. One such example is a human IgG4 constant region. In one embodiment, the IgG4 antibody comprises a modification to prevent the formation of half antibodies (fab arm exchange) in vivo, e.g., the antibody comprises an IgG4 heavy chain comprising a serine to proline mutation in residue 241, corresponding to position 228 according to the EU-index (Kabat et al., “Sequences of proteins of immunological interest”, 5^{th} ed., NIH, Bethesda, M L, 1991). Such modified IgG4 antibodies will remain intact in vivo and maintain a bivalent (high affinity) binding to NKG2A, as opposed to native IgG4 that will undergo fab arm exchange in vivo such that they bind to NKG2A in monovalent manner which can alter binding affinity. Alternatively, antibody fragments that do not comprise constant regions, such as Fab or F(ab)2 fragments, can be used to avoid Fc receptor binding. Fc receptor binding can be assessed according to methods known in the art, including for example testing binding of an antibody to Fc receptor protein in a BIACORE assay. Also, any human antibody type (e.g. IgG1, IgG2, IgG3 or IgG4) can be used in which the Fc portion is modified to minimize or eliminate binding to Fc receptors (see, e.g., WO03101485, the disclosure of which is herein incorporated by reference). Assays such as, e.g., cell based assays, to assess Fc receptor binding are well known in the art, and are described in, e.g., WO03101485.

In one aspect of the invention, the agent reduces CD94/NKG2A-mediated inhibition of a CD94/NKG2A-expressing lymphocyte by interfering with CD94/NKG2A signaling by, e.g., interfering with the binding of HLA-E by NKG2A, preventing or inducing conformational changes in the CD94/NKG2A receptor, and/or affecting dimerization and/or clustering of the CD94/NKG2A receptor.

In one embodiment, an anti-NKG2A antibody will not bind to human NKG2C, NKG2E and/or NKG2H (e.g. when tested at a concentration of 10 μg/ml), or will bind to NKG2C and E with substantially decreased affinity compared to its ability to bind NKG2A. In one aspect, the antibody binds to NKG2A with a KD at least 100-fold lower than to human NKG2C. In one aspect, the antibody binds to NKG2A with a KD at least 100-fold lower than to human NKG2E.

In one aspect of the invention, the agent binds to an extracellular portion of NKG2A with a KD at least 100 fold lower than to NKG2C. In a further preferred aspect, the agent binds to an extracellular portion of NKG2A with a KD at least 150, 200, 300, 400, or 10,000 fold lower than to NKG2C. In another aspect of the invention, the agent binds to an extracellular portion of NKG2A with a KD at least 100 fold lower than to NKG2C, NKG2E and/or NKG2H molecules. In a further preferred aspect, the agent binds to an extracellular portion of NKG2A with a KD at least 150, 200, 300, 400, or 10,000 fold lower than to NKG2C, NKG2C and/or NKG2H molecules. This can be measured, for instance, in BiaCore experiments, in which the capacity of agents to bind the extracellular portion of immobilized CD94/NKG2A (e.g. purified from CD94/NKG2 expressing cells, or produced in a bio-system) is measured and compared to the binding of agents to similarly produced CD94/NKG2C and/or other CD94/NKG2 variants in the same assay. Alternatively, the binding of agents to cells that either naturally express, or over-express (e.g. after transient or stable transfection), CD94/NKG2A can be measured and compared to binding of cells expressing CD94/NKG2C and/or other CD94/NKG2 variants. Anti-NKG2A antibodies may optionally bind NKG2B, which is an NKG2A splice variant forming an inhibitory receptor together with CD94. In one embodiment, affinity can be measured using the methods disclosed in U.S. Pat. No. 8,206,709, for example by assessing binding to covalently immobilized NKG2A-CD94-Fc fusion protein by Biacore as shown in Example 8 of U.S. Pat. No. 8,206,709, the disclosure of which is incorporated herein by reference.

In any aspect, comparative binding to NKG2A, NKG2C, NKG2E and/or NKG2H is assessed at a concentration of 5-10 μg/ml, optionally about 10 μg/ml.

The anti-NKG2A antibody can be a humanized antibody, for example comprising a VH human acceptor framework from a human acceptor sequence selected from, e.g., VH1_18, VH5_a, VH5_51, VH1_f, and VH1_46, and a JH6 J-segment, or other human germline VH framework sequences known in the art. The VL region human acceptor sequence may be, e.g., VKI_O2/JK4.

In one embodiment, the antibody is a humanized antibody based on antibody Z270. Different humanized Z270 heavy chain variable regions are shown in SEQ ID NOS: 68-72, with optionally further comprising a C-terminal serine (S) residue. The HumZ270VH6 variable region of SEQ ID NO: 68 is based on a human VH5_51 gene; the HumZ270VH1 variable region of SEQ ID NO: 69 is based on a human VH1_18 gene; the humZ270VH5 variable region of SEQ ID NO: 70 is based on a human VH5_a gene; the humZ270VH7 variable region of SEQ ID NO: 71 is based on a human VH1_f gene; and the humZ270VH8 variable region of SEQ ID NO: 72 is based on a human VH1_46 gene; all with a human JH6 J-segment. Each of these antibodies retains high affinity binding to NKG2A, with low likelihood of a host immune response against the antibody as the 6 C-terminal amino acid residues of the Kabat H-CDR2 of each of the humanized constructs are identical to the human acceptor framework. Using the alignment program VectorNTI, the following sequence identities between humZ270VH1 and humZ270VH5, -6, -7, and -8 were obtained: 78.2% (VH1 vs. VH5), 79.0% (VH1 vs. VH6), 88.7% (VH1 vs. VH7), and 96.0% (VH1 vs. VH8).

In one aspect, the agent comprises (i) a heavy chain variable region amino acid sequence of SEQ ID NOS: 68-72, or an amino acid sequence at least 50%, 60%, 70%, 80%, 90%, 95%, 98% or 99% identical thereto, and (ii) the light chain variable region amino acid sequence of SEQ ID NO: 73, or an amino acid sequence at least 50%, 60%, 70%, 80%, 90%, 95%, 98% or 99% identical thereto. In one aspect, the agent comprises (i) a heavy chain amino acid sequence of SEQ ID NOS: 74-78, or an amino acid sequence at least 50%, 60%, 70%, 80%, 90%, 95%, 98% or 99% identical thereto, and (ii) a light chain amino acid sequence of SEQ ID NO: 79, or an amino acid sequence at least 50%, 60%, 70%, 80%, 90%, 95%, 98% or 99% identical thereto.

The antibody having a heavy chain variable region amino acid sequence of any of SEQ ID NOS: 68-72 and a light chain variable region amino acid sequence of SEQ ID NO: 73 neutralizes the inhibitory activity of NKG2A, but does not substantially bind the activating receptors NKG2C, NKG2E or NKG2H. This antibody furthermore competes with HLA-E for binding to NKG2A on the surface of a cell. In one aspect, the agent comprises H-CDR1, H-CDR2 and/or H-CDR3 sequences derived from the heavy chain variable region amino acid sequence of SEQ ID NOS: 68-72. In one aspect of the invention, the agent comprises L-CDR1, L-CDR2 and/or L-CDR3 sequences derived from the light chain variable region amino acid sequence of SEQ ID NO: 73.

Heavy chain variable regions
VH6:
(SEQ ID NO: 68)
EVQLVQSGAEVKKPGESLKISCKGSGYSFTSYWMNWVRQMPGKGLEWMGRIDPYD
SETHYSPSFQGQVTISADKSISTAYLQWSSLKASDTAMYYCARGGYDFDVGTLY
WFFDVWGQGTTVTVS
VH1:
(SEQ ID NO: 69)
QVQLVQSGAEVKKPGASVKVSCKASGYTFTSYWMNWVRQAPGQGLEWMGRIDPYDSETHYA
QKLQGRVTMTTDTSTSTAYMELRSLRSDDTAVYYCARGGYDFDVGTLYWFFDVWGQGTTVTVS
VH5:
(SEQ ID NO: 70)
EVQLVQSGAEVKKPGESLRISCKGSGYSFTSYWMNWVRQMPGKGLEWMGRIDPYD
SETHYSPSFQGHVTISADKSISTAYLQWSSLKASDTAMYYCARGGYDFDVGTLY
WFFDVWGQGTTVTVS
VH7:
(SEQ ID NO: 71)
EVQLVQSGAEVKKPGATVKISCKVSGYTFTSYWMNWVQQAPGKGLEWMGRIDPYDSETHY
AEKFQGRVTITADTSTDTAYMELSSLRSEDTAVYYCATGGYDFDVGTLYWFFDVWGQGTTVTVS
VH8:
(SEQ ID NO: 72)
QVQLVQSGAEVKKPGASVKVSCKASGYTFTSYWMNWVRQAPGQGLEWMGRIDPYDSETHY
AQKFQGRVTMTRDTSTSTVYMELSSLRSEDTAVYYCARGGYDFDVGTLYWFFDVWGQGTTVTVS
Light chain variable region
(SEQ ID NO: 73)
DIQMTQSPSSLSASVGDRVTITCRASENIYSYLAWYQQKPGKAPKLLIYNAKTLAEGVPSRFSGS
GSGTDFTLTISSLQPEDFATYYCQHHYGTPRTFGGGTKVEIK
Heavy Chains (variable region domain amino acids underlined)
VH6:
(SEQ ID NO: 74)
EVQLVQSGAEVKKPGESLKISCKGSGYSFTSYWMNWVRQMPGKGLEWMGRIDPYD
SETHYSPSFQGQVTISADKSISTAYLQWSSLKASDTAMYYCARGGYDFDVGTLY
WFFDVWGQGTTVTVSSASTKGPSVFPLAPCSRSTSESTAALGCLVKDYFPEPVTVSWNSGALTSGV
HTFPAVLQSSGLYSLSSVVTVPSSSLGTKTYTCNVDHKP
SNTKVDKRVESKYGPPCPPCPAPEFLGGPSVFLFPPKPKDTLMIS
RTPEVTCVVVDVSQEDPEVQFNWYVDGVEVHNAK
TKPREEQFNSTYRVVSVLTVLHQDWLNGKEYKCKVSNKGLPSSIEK
TISKAKGQPREPQVYTLPPSQEEMTKNQVSLTCLVKGFYPSDIAVEWESNGQPEN
NYKTTPPVLDSDGSFFLYSRLTVDKSRWQEGNVFSCSVMHEALHNHYTQKSLSLSLGK
VH1:
(SEQ ID NO: 75)
QVQLVQSGAEVKKPGASVKVSCKASGYTFTSYWMNWVRQAPGQGLEWMGRIDPYDSETHYA
QKLQGRVTMTTDTSTSTAYMELRSLRSDDTAVYYCARGGYDFDVGTLYWFFDVWGQGTTVTVS
SASTKGPSVFPLAPCSRSTSESTAALGCLVKDYFPEPVTVSWNSGALTSGVHTFPAVLQSSGLYS
LSSVVTVPSSSLGTKTYTCNVDHKPSNTKVDKRVESKYGPPCPPCPAPEFLGGPSVFLFPPKPKD
TLMISRTPEVTCVVVDVSQEDPEVQFNWYVDGVEVHNAKTKPREEQFNSTYRVVSVLTVLHQDW
LNGKEYKCKVSNKGLPSSIEKTISKAKGQPREPQVYTLPPSQEEMTKNQVSLTCLVKGFYPSDIAV
EWESNGQPENNYKTTPPVLDSDGSFFLYSRLTVDKSRWQEGNVFSCSVMHEALHNHYTQKSLS
LSLGK
VH5:
(SEQ ID NO: 76)
EVQLVQSGAEVKKPGESLRISCKGSGYSFTSYWMNWVRQMPGKGLEWMGRIDPYD
SETHYSPSFQGHVTISADKSISTAYLQWSSLKASDTAMYYCARGGYDFDVGTLY
WFFDVWGQGTTVTVSSASTKGPSVFPLAPCSRSTSESTAALGCLVKDYFPEPVTVSWNSGALTSGV
HTFPAVLQSSGLYSLSSVVTVPSSSLGTKTYTCNVDHKP
SNTKVDKRVESKYGPPCPPCPAPEFLGGPSVFLFPPKPKDTLMIS
RTPEVTCVVVDVSQEDPEVQFNWYVDGVEVHNAKTKPREEQFNSTYRVVSVLTVLHQDW
LNGKEYKCKVSNKGLPSSIEKTISKAKGQPREPQVYTLPPSQEEMTKNQVSLTCLVKGFYPSDIAV
EWESNGQPENNYKTTPPVLDSDGSFFLYSRLTVDKSRWQEGNVFSCSVMHEALHNHYTQKSLS
LSLGK
VH7:
(SEQ ID NO: 77)
EVQLVQSGAEVKKPGATVKISCKVSGYTFTSYWMNWVQQAPGKGLEWMGRIDPYDSETHY
AEKFQGRVTITADTSTDTAYMELSSLRSEDTAVYYCATGGYDFDVGTLY
WFFDVWGQGTTVTVSSASTKGPSVFPLAPCSRSTSESTAALGCLVKDYFPEPVTVSWNSGALTSGV
HTFPAVLQSSGLYSLSSVVTVPSSSLGTKTYTCNVDHKP
SNTKVDKRVESKYGPPCPPCPAPEFLGGPSVFLFPPKPKDTLMIS
RTPEVTCVVVDVSQEDPEVQFNWYVDGVEVHNAKTKPREEQFNSTYRVVSVLTVLHQDW
LNGKEYKCKVSNKGLPSSIEKTISKAKGQPREPQVYTLPPSQEEMTKNQVSLTCLVKGFYPSDIAV
EWESNGQPENNYKTTPPVLDSDGSFFLYSRLTVDKSRWQEGNVFSCSVMHEALHNHYTQKSLS
LSLGK
VH8:
(SEQ ID NO: 78)
QVQLVQSGAEVKKPGASVKVSCKASGYTFTSYWMNWVRQAPGQGLEWMGRIDPYDSETHY
AQKFQGRVTMTRDTSTSTVYMELSSLRSEDTAVYYCARGGYDFDVGTLYWFFDVWGQGTTVTVS
SASTKGPSVFPLAPCSRSTSESTAALGCLVKDYFPEPVTVSWNSGALTSGVHT
FPAVLQSSGLYSLSSVVTVPSSSLGTKTYTCNVDHKPSNTKVDKRVESKYGPPCPPCPAPE
FLGGPSVFLFPPKPKDTLMISRTPEVTCVVVDVSQEDPEVQFNWYVDGVEVHNAK
TKPREEQFNSTYRVVSVLTVLHQDWLNGKEYKCKVSNKGLPSSIEK
TISKAKGQPREPQVYTLPPSQEEMTKNQVSLTCLVKGFYPSDIAVEWESNGQPEN
NYKTTPPVLDSDGSFFLYSRLTVDKSRWQEGNVFSCSVMHEALHNHYTQKSLSLSLGK
Light chain (variable region domain amino acids underlined)
(SEQ ID NO: 79)
DIQMTQSPSSLSASVGDRVTITCRASENIYSYLAWYQQKPGKAPKLLIYNAKTLAEGVPSRFSGS
GSGTDFTLTISSLQPEDFATYYCQHHYGTPRTFGGGTKVEIKRTVAAPSVFIFPPSDEQLKSGTASVVC
LLNNFYPREAKVQWKVDNALQSGNSQESVTEQDSKDSTYSLSSTLTLSKADYEKHKVYACEVTHQG
LSSPVTKSFNRGEC
Monalizumab heavy and light chain CDRs
Heavy chain CDRs, according to Kabat numbering scheme:
H-CDR1:
(SEQ ID NO: 80)
SYWMN
H-CDR2:
(SEQ ID NO: 81)
RIDPYDSETHYAQKLQG
H-CDR3:
(SEQ ID NO: 82)
GGYDFDVGTLYWFFDV
Light chain CDRs according to Kabat numbering scheme:
L-CDR1:
(SEQ ID NO: 83)
RASENIYSYLA
L-CDR2:
(SEQ ID NO: 84)
NAKTLAE
L-CDR3:
(SEQ ID NO: 85)
QHHYGTPRT

In one aspect, the anti-NKG2A antibody is an antibody comprising a H-CDR1 corresponding to residues 31-35 of SEQ ID NOS: 68-72 (or of SEQ ID NOS: 74-78), a H-CDR2 corresponding to residues 50-60 (optionally 50-66 when including amino acids of human origin) of SEQ ID NOS: 68-72 (or of SEQ ID NOS: 74-78), and a H-CDR3 corresponding to residues 99-114 (95-102 according to Kabat) of SEQ ID NOS: 68-72 (or of SEQ ID NOS: 74-78). In one embodiment, the H-CDR2 corresponding to residues 50-66 of SEQ ID NOS: 68-72 (or of SEQ ID NOS: 74-78). Optionally, a CDR may comprise one, two, three, four, or more amino acid substitutions.

In one aspect, the anti-NKG2A antibody is an antibody comprising a L-CDR1 corresponding to residues 24-34 of SEQ ID NOS: 73 or 79, a L-CDR2 corresponding to residues 50-56 of SEQ ID NOS: 73 or 79, and an L-CDR3 corresponding to residues 89-97 of SEQ ID NOS: 73 or 79. Optionally, a CDR may comprise one, two, three, four, or more amino acid substitutions.

In one aspect, the anti-NKG2A antibody is an antibody comprising a H-CDR1 corresponding to residues 31-35 of SEQ ID NOS: 68-72, a H-CDR2 corresponding to residues 50-60 (optionally 50-66) of SEQ ID NOS: 68-72, and a H-CDR3 corresponding to residues 99-114 (95-102 according to Kabat) of SEQ ID NOS: 68-72, a L-CDR1 corresponding to residues 24-34 of SEQ ID NO: 73, a L-CDR2 corresponding to residues 50-56 of SEQ ID NO: 73, and an L-CDR3 corresponding to residues 89-97 of SEQ ID NO: 73.

In one aspect, the anti-NKG2A antibody is an antibody comprising the heavy chain H-CDR1, H-CDR2 and H-CDR3 domains having the amino acid sequences of SEQ ID NOS: 80-82, and the light chain L-CDR1, L-CDR2 and L-CDR3 domains having the amino acid sequences of SEQ ID NOS: 83-85, respectively.

In one aspect, the agent is monalizumab, an anti-NKG2A antibody having the heavy chain variable region amino acid sequence of SEQ ID NO: 69 and the light chain variable region amino acid sequence of SEQ ID NO: 73. In one aspect, the agent is monalizumab, an anti-NKG2A antibody having the heavy chain amino acid sequence of SEQ ID NO: 75 and the light chain amino acid sequence of SEQ ID NO: 79.

In one aspect, the agent is BMS-986315 (Bristol Myers Squibb Corp., New York, NY), or an antibody disclosed in PCT publication no. WO2020/102501 the disclosure of which is incorporated herein by reference, an antibody blocking the interaction between NKG2A and HLA-E. In one aspect, the agent comprises the heavy and light chain CDR1, CDR2 and/or CDR3 of BMS-986315. In one aspect, the anti-NKG2A antibody comprises the following heavy and light chain CDR amino acid sequences:

HCDR1:
(SEQ ID NO: 86)
SHSMN
HCDR2:
(SEQ ID NO: 87)
AISSSSSYIYYADSVKG
HCDR3:
(SEQ ID NO: 88)
EEWGLPFDY
LCDR1:
(SEQ ID NO: 89)
RASQGISSALA,
(SEQ ID NO: 90)
RASQGIPSALA,
or
(SEQ ID NO: 91)
RASQGINSALA
LCDR2:
(SEQ ID NO: 92)
DASSLKS
LCDR3:
(SEQ ID NO: 93)
QQFNSYPLT

In one aspect, the agent is an antibody disclosed in PCT publication no. WO2020/094071 the disclosure of which is incorporated herein by reference, or an antibody comprising the heavy and light chain CDRs thereof. In one aspect, the agent comprises the heavy and light chain CDR1, CDR2 and/or CDR3 of M15-5, Mpb416, Mab031, Mab032 or Mab033.

In one aspect, the anti-NKG2A antibody comprises the following M15-5 heavy and light chain CDR amino acid sequences:

HCDR1:
(SEQ ID NO: 94)
NTYIH
HCDR2:
(SEQ ID NO: 95)
IDPANADTKYAPTFQG
HCDR3:
(SEQ ID NO: 96)
YRDYLFYYALGY
LCDR1:
(SEQ ID NO: 97)
RSSKSLLHSNANTYLY
LCDR2:
(SEQ ID NO: 98)
RMSNLAS
LCDR3:
(SEQ ID NO: 99)
MQHLEYPYT

In one aspect, the anti-NKG2A antibody comprises the following Mpb416 heavy and light chain CDR amino acid sequences:

HCDR1:
(SEQ ID NO: 94)
NTYIH
HCDR2:
(SEQ ID NO: 100)
IDPANGDTKYAPTFQG
HCDR3:
(SEQ ID NO: 96)
YRDYLFYYALGY
LCDR1:
(SEQ ID NO: 101)
RSSKSLLHSNGNTYLY
LCDR2:
(SEQ ID NO: 98)
RMSNLAS
LCDR3:
(SEQ ID NO: 99)
MQHLEYPYT.

In one aspect, the anti-NKG2A antibody comprises the following Mab031 heavy and light chain CDR amino acid sequences:

HCDR1:
(SEQ ID NO: 94)
NTYIH
HCDR2:
(SEQ ID NO: 102)
IDPANGDTKYAPKFQG
HCDR3:
(SEQ ID NO: 103)
YGNYLYYYSLDY
LCDR1:
(SEQ ID NO: 101)
RSSKSLLHSNGNTYLY
LCDR2:
(SEQ ID NO: 98)
RMSNLAS
LCDR3:
(SEQ ID NO: 99)
MQHLEYPYT

In one aspect, the anti-NKG2A antibody comprises the following Mab032 heavy and light chain CDR amino acid sequences:

HCDR1:
(SEQ ID NO: 104)
NTYMH
HCDR2:
(SEQ ID NO: 105)
IDPADGDTQYAPKFQG
HCDR3:
(SEQ ID NO: 106)
YGNYLFYYSMDY
LCDR1:
(SEQ ID NO: 101)
RSSKSLLHSNGNTYLY
LCDR2:
(SEQ ID NO: 98)
RMSNLAS
LCDR3:
(SEQ ID NO: 99)
MQHLEYPYT.

In one aspect, the anti-NKG2A antibody comprises the following Mab033 heavy and light chain CDR amino acid sequences:

HCDR1:
(SEQ ID NO: 94)
NTYIH
HCDR2:
(SEQ ID NO: 107)
IDPANGDTQYDPKFQG
HCDR3:
(SEQ ID NO: 108)
YGDYLFYYSLKY
LCDR1:
(SEQ ID NO: 101)
RSSKSLLHSNGNTYLY
LCDR2:
(SEQ ID NO: 98)
RMSNLAS
LCDR3:
(SEQ ID NO: 109)
MQHLESPYT.

In one aspect, the agent is Z199, an antibody that neutralizes NKG2A without blocking the interaction between NKG2A and HLA-E. In one aspect, the agent comprises the heavy and light chain CDR1, CDR2 and/or CDR3 of Z199.

In one aspect, the agent comprises H-CDR1, H-CDR2 and/or H-CDR3 sequences derived from the Z199 VH having the amino acid sequence of SEQ ID NO: 110, e.g. according to Kabat numbering (see CDRs underlined in SEQ ID NO: 110, below). In one aspect of the invention, the agent comprises L-CDR1, L-CDR2 and/or L-CDR3 sequences derived from the Z199 VL having the amino acid sequence of SEQ ID NO: 111, e.g. according to Kabat numbering (see CDRs underlined in SEQ ID NO: 111, below). In one aspect, the agent comprises H-CDR1, H-CDR2 and/or H-CDR3 sequences derived from the VH having the amino acid sequence of SEQ ID NO: 110, and L-CDR1, L-CDR2 and/or L-CDR3 sequences derived from the VL having the amino acid sequence of SEQ ID NO: 111. The antibody having the heavy chain variable region of SEQ ID NO: 110 and a light chain variable region of SEQ ID NO: 111 neutralizes the inhibitory activity of NKG2A, and also binds the activating receptors NKG2C, NKG2E and NKG2H. This antibody does not compete with HLA-E for binding to NKG2A on the surface of a cell (i.e. it is a non-competitive antagonist of NKG2A).

(SEQ ID NO: 110)
EVQLVESGGGLVKPGGSLKLSCAASGFTFSSYAMSWVRQS
PEKRLEWVAEISSGGSYTYYPDTVTGRFTISRDNAKNTLY
LEISSLRSEDTAMYYCTRHGDYPRFFDVWGAGTTVTVSS
(SEQ ID NO: 111)
QIVLTQSPALMSASPGEKVTMTCSASSSVSYIYWYQQKPR
SSPKPWIYLTSNLASGVPARFSGSGSGTSYSLTISSMEAE
DAATYYCQQWSGNPYTFGGGTKLEIK

In one aspect, the agent comprises amino acid residues 31-35, 50-60, 62, 64, 66, and 99-108 of the variable-heavy (V_{H}) domain of SEQ ID NO: 110 and amino acid residues 24-33, 49-55, and 88-96 of the variable-light (V_{L}) domain of SEQ ID NO: 111, optionally with one, two, three, four, or more amino acid substitutions. In one aspect, the agent is a humanized antibody, for example an agent comprising heavy and light chain variable regions as disclosed in PCT publication no. WO2009/092805, the disclosure of which is incorporated herein by reference.

In one aspect, the agent is a fully human antibody which has been raised against the CD94/NKG2A epitope to which any of the aforementioned antibodies bind.

It will be appreciated that, while the aforementioned antibodies can be used, other antibodies can recognize and be raised against any part of the NKG2A polypeptide so long as the antibody causes the neutralization of the inhibitory activity of NKG2A. For example, any fragment of NKG2A, preferably but not exclusively human NKG2A, or any combination of NKG2A fragments, can be used as immunogens to raise antibodies, and the antibodies can recognize epitopes at any location within the NKG2A polypeptide, so long as they can do so on NKG2A expressing NK cells as described herein. Optionally, the epitope is the epitope specifically recognized by an antibody having a heavy chain variable region of SEQ ID NOS: 68-72 and a light chain variable region of SEQ ID NO: 73.

In one aspect, the agent is an antibody that is a function-conservative variant of humZ270 or of an antibody having a heavy chain of SEQ ID NO: 75 and a light chain of SEQ ID NO: 79. “Function-conservative variants” are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide which has at least 60% amino acid identity as determined by BLAST or FASTA algorithms, preferably at least 75%, more preferably at least 85%, still preferably at least 90%, and even more preferably at least 95%, and which has the same or substantially similar properties or functions as the native or parent protein to which it is compared.

In one aspect, the agent competes with humZ270 antibody disclosed in U.S. Pat. No. 8,206,709 (the disclosure of which is incorporated herein by reference) in binding to the extra-cellular portion of human CD94/NKG2A receptor. Competitive binding can be measured, for instance, in BiaCore experiments, in which the capacity of agents is measured, for binding the extracellular portion of immobilized CD94/NKG2A receptor (e.g. purified from CD94/NKG2 expressing cells, or produced in a bio-system) saturated with humZ270. Alternatively, the binding of agents to cells is measured that either naturally express, or over-express (e.g. after transient or stable transfection), CD94/NKG2A receptor, and which have been pre-incubated with saturating doses of Z270. In one embodiment, competitive binding can be measured using the methods disclosed in U.S. Pat. No. 8,206,709, for example by assessing binding to Ba/F3-CD94-NKG2A cells by flow cytometry as shown in Example 15 of U.S. Pat. No. 8,206,709, the disclosure of which is incorporate herein by reference.

### Anti-ILT2 Antibodies

An anti-ILT-2 antibody that neutralizes the inhibitory activity of ILT-2 binds an extra-cellular portion of human ILT-2 receptor and reduces the inhibitory activity of human ILT2 receptor expressed on the surface of an ILT2 positive cell, e.g. an NK cell. In one embodiment the agent competes with HLA-G in binding to ILT-2, i.e. the agent blocks the interaction between ILT-2 and an HLA ligand thereof (e.g. HLA-G).

The starting point for anti-ILT2 antibodies that can then be tested for ILT-2 neutralization activity can include any known antibodies, e.g. GHI/75, 292319, HP-F1, 586326 or 292305, or new antibodies produced by classical immunization protocols (e.g. in mice or rats) or selected from libraries of immunoglobulins or immunoglobulin sequences, as disclosed for instance in (Ward et al. Nature, 341 (1989) p. 544). Antibodies can be titrated on ILT2 proteins for the concentration required to achieve maximal binding to a ILT2 polypeptide. Once antibodies are identified that are capable of binding ILT2 and/or having other desired properties, they will also typically be assessed, using standard methods including those described herein, for their ability to bind to other polypeptides, including other ILT2 polypeptides and/or unrelated polypeptides. Ideally, the antibodies only bind with substantial affinity to ILT2 and do not bind at a significant level to unrelated polypeptides or to other ILT proteins, notably ILT-1, -3, -4, -5, -6, -7, and/or -8). However, it will be appreciated that, as long as the affinity (e.g., KD as determined by SPR) for ILT2 is substantially greater (e.g., 10×, 100×, 1000×, 10,000×, or more) than it is for other ILTs and/or other, unrelated polypeptides), then the antibodies are suitable for use in the present methods.

Ideally, the antibodies only bind with substantial affinity to ILT2 and do not bind at a significant level to unrelated polypeptides or to other ILT proteins, notably ILT-1, -3, -4, -5, -6, -7, and/or -8). However, it will be appreciated that, as long as the affinity (e.g., KD as determined by SPR) for ILT2 is substantially greater (e.g., 10×, 100×, 1000×, 10,000×, or more) than it is for other ILTs and/or other, unrelated polypeptides), then the antibodies are suitable for use in the present methods.

In any embodiment herein, an antibody can be characterized by a KD for binding affinity of less than 1×10⁻⁸ M, optionally less than 1×10⁻⁹ M, or of about 1×10⁻⁸ M to about 1×10⁻¹⁰ M, or about 1×10⁻⁹ M to about 1×10⁻¹¹ M, for binding to a human a human ILT2 polypeptide. In one embodiment, affinity is monovalent binding affinity. In one embodiment, affinity is bivalent binding affinity.

In any embodiment herein, an antibody can be characterized by a monovalent KD for binding affinity of less than 2 nM, optionally less than 1 nM.

In any embodiment herein, an antibody can be characterized by a 1:1 Binding fit, as determined by SPR. In any embodiment herein, an antibody can be characterized by dissociation or off rate (kd (1/s)) of less than about 1E-2, optionally less than about 1E-3.

The anti-ILT2 antibodies can be prepared as non-depleting antibodies such that they have reduced, or substantially lack, specific binding to human Fcγ receptors. Such antibodies may comprise constant regions of various heavy chains that are known not to bind, or to have low binding affinity for CD16 and optionally further other Fcγ receptors. One such example is a wild-type human IgG4 constant region which naturally has lowered CD16 binding but retains significant binding to other receptors such as CD64. Alternatively, antibody fragments that do not comprise constant regions, such as Fab or F(ab′)2 fragments, can be used to avoid Fc receptor binding. Fc receptor binding can be assessed according to methods known in the art, including for example testing binding of an antibody to Fc receptor protein in a BIACORE assay. Also, any antibody isotype (e.g. human IgG1, IgG2, IgG3 or IgG4) can be used in which the Fc portion is modified to decrease, minimize or eliminate binding to Fc receptors (see, e.g., WO03101485). Assays such as, e.g., cell based assays, to assess Fc receptor binding are well known in the art, and are described in, e.g., WO03101485.

Cross-blocking assays can also be used to evaluate whether a test antibody affects the binding of the HLA class I ligand for human ILT2. For example, to determine whether an anti-ILT2 antibody preparation reduces or blocks ILT2 interactions with an HLA class I molecule, the following test can be performed: A dose-range of anti-human ILT2 Fab is co-incubated 30 minutes at room temperature with the human ILT2-Fc at a fixed dose, then added on HLA class I-ligand expressing cell lines for 1 h. After washing cells two times in staining buffer, a PE-coupled goat anti-mouse IgG Fc fragment secondary antibodies diluted in staining buffer is added to the cells and plates are incubated for 30 additional minutes at 4° C. Cells are washed two times and analyzed on an Accury C6 flow cytometer equipped with an HTFC plate reader. In the absence of test antibodies, the ILT2-Fc binds to the cells. In the presence of an antibody preparation pre-incubated with ILT2-Fc that blocks ILT2-binding to HLA class I, there is a reduced binding of ILT2-Fc to the cells.

In one aspect, the antibodies lack binding to an ILT2 protein modified to lack the D1 domain. In one aspect, the antibodies bind full-length wild-type ILT2 polypeptide but lack binding to an ILT2 protein modified to lack the segment of residues 24 to 121 of the amino acid sequence of SEQ ID NO: 1. In another aspect, the antibodies bind full-length wild-type ILT2 polypeptide but have reduced binding to an ILT2 protein modified to lack the D4 domain. In one aspect, the antibodies bind full-length wild-type ILT2 polypeptide but lack binding to an ILT2 protein modified to lack the segment of residues 322 to 458 of the amino acid sequence of SEQ ID NO: 1.

Binding of anti-ILT2 antibody to cells transfected to express a ILT2 mutant can be measured and compared to the ability of anti-ILT2 antibody to bind cells expressing wild-type ILT2 polypeptide (e.g., SEQ ID NO: 1). A reduction in binding between an anti-ILT2 antibody and a mutant ILT2 polypeptide means that there is a reduction in binding affinity (e.g., as measured by known methods such FACS testing of cells expressing a particular mutant, or by Biacore™ (SPR) testing of binding to mutant polypeptides) and/or a reduction in the total binding capacity of the anti-ILT antibody (e.g., as evidenced by a decrease in Bmax in a plot of anti-ILT2 antibody concentration versus polypeptide concentration). A significant reduction in binding indicates that the mutated residue is directly involved in binding to the anti-ILT2 antibody or is in close proximity to the binding protein when the anti-ILT2 antibody is bound to ILT2.

In some embodiments, a significant reduction in binding means that the binding affinity and/or capacity between an anti-ILT2 antibody and a mutant ILT2 polypeptide is reduced by greater than 40%, greater than 50%, greater than 55%, greater than 60%, greater than 65%, greater than 70%, greater than 75%, greater than 80%, greater than 85%, greater than 90% or greater than 95% relative to binding between the antibody and a wild type ILT2 polypeptide. In certain embodiments, binding is reduced below detectable limits. In some embodiments, a significant reduction in binding is evidenced when binding of an anti-ILT2 antibody to a mutant ILT2 polypeptide is less than 50% (e.g., less than 45%, 40%, 35%, 30%, 25%, 20%, 15% or 10%) of the binding observed between the anti-ILT2 antibody and a wild-type ILT2 polypeptide.

Once an antigen-binding compound having the desired binding for ILT2 is obtained it may be assessed for its ability to inhibit ILT2. For example, if an anti-ILT2 antibody reduces or blocks ILT2 activation induced by a HLA ligand (e.g., as present on a cell), it can increase the cytotoxicity of ILT2-restricted lymphocytes. This can be evaluated by a typical cytotoxicity assay, examples of which are described below.

The ability of an antibody to reduce ILT2-mediated signaling can be tested in a standard 4-hour in vitro cytotoxicity assay using, e.g., NK cells that express ILT2, and target cells that express an HLA ligand of the ILT2. Such NK cells do not efficiently kill targets that express the ligand because ILT2 recognizes the HLA ligand, leading to initiation and propagation of inhibitory signaling that prevents lymphocyte-mediated cytolysis. Such an assay can be carried out using primary NK cells, e.g., fresh NK cells purified from donors, incubated overnight at 37° C. before use. Such an in vitro cytotoxicity assay can be carried out by standard methods that are well known in the art, as described for example in Coligan et al., eds., Current Protocols in Immunology, Greene Publishing Assoc. and Wiley Interscience, N.Y., (1992, 1993). The target cells are labeled with ⁵¹Cr prior to addition of NK cells, and then the killing is estimated as proportional to the release of ⁵¹Cr from the cells to the medium, as a result of killing. The addition of an antibody that prevents ILT2 protein from binding to the HLA class I ligand (e.g. HLA-G) results in prevention of the initiation and propagation of inhibitory signaling via the ILT2 protein. Therefore, addition of such agents results in increases in lymphocyte-mediated killing of the target cells. This step thereby identifies agents that prevent ILT2-mediated negative signaling by, e.g., blocking ligand binding. In a particular ⁵¹Cr-release cytotoxicity assay, ILT2-expressing NK effector-cells can kill HLA ligand-negative target cells, but less well HLA ligand-expressing control cells. Thus, NK effector cells kill less efficiently HLA ligand positive cells due to HLA-induced inhibitory signaling via ILT2. When NK cells are pre-incubated with blocking anti-ILT2 antibodies in such a ⁵¹Cr-release cytotoxicity assay, HLA ligand-expressing cells are more efficiently killed, in an antibody-concentration-dependent fashion.

The inhibitory activity (i.e., cytotoxicity enhancing potential) of an antibody can also be assessed in any of a number of other ways, e.g., by its effect on intracellular free calcium as described, e.g., in Sivori et al., J. Exp. Med. 1997; 186:1129-1136, the disclosure of which is herein incorporated by reference, or by the effect on markers of NK cell cytotoxicity activation, such as degranulation marker CD107 or CD137 expression. NK or CD8 T cell activity can also be assessed using any cell based cytotoxicity assays, e.g., measuring any other parameter to assess the ability of the antibody to stimulate NK cells to kill target cells such as P815, K562 cells, or appropriate tumor cells as disclosed in Sivori et al., J. Exp. Med. 1997; 186:1129-1136; Vitale et al., J. Exp. Med. 1998; 187:2065-2072; Pessino et al. J. Exp. Med. 1998; 188:953-960; Neri et al. Clin. Diag. Lab. Immun. 2001; 8:1131-1135; Pende et al. J. Exp. Med. 1999; 190:1505-1516, the entire disclosures of each of which are herein incorporated by reference.

In one embodiment, an antibody preparation causes at least a 10% augmentation in the cytotoxicity of an ILT2-restricted lymphocyte, preferably at least a 30%, 40% or 50% augmentation in NK cytotoxicity, or more preferably at least a 60% or 70% augmentation in NK cytotoxicity.

The activity of a cytotoxic lymphocyte can also be addressed using a cytokine-release assay, wherein NK cells are incubated with the antibody to stimulate the cytokine production of the NK cells (for example IFN-γ and TNF-α production). In an exemplary protocol, IFN-γ production from PBMC is assessed by cell surface and intracytoplasmic staining and analysis by flow cytometry after 4 days in culture. Briefly, Brefeldin A (Sigma Aldrich) is added at a final concentration of 5 μg/ml for the last 4 hours of culture. The cells are then incubated with anti-CD3 and anti-CD56 mAb prior to permeabilization (IntraPrep™; Beckman Coulter) and staining with PE-anti-IFN-γ or PE-IgG1 (Pharmingen). GM-CSF and IFN-γ production from polyclonal activated NK cells are measured in supernatants using ELISA (GM-CSF: DuoSet Elisa, R&D Systems, Minneapolis, MN, IFN-γ: OptEIA set, Pharmingen).

In one approach, antibodies can optionally be identified and selected based on binding to the same region or epitope on the surface of the ILT2 polypeptide as any known antibody, for example any of the exemplary antibodies described herein, e.g., 48F12, 3F5, 2H2A, 12D12, 26D8 or 18E1 (e.g. an epitope- or binding region-directed screen). In one aspect, the antibodies bind substantially the same epitope as any of antibodies 48F12, 3F5, 2H2A, 12D12, 26D8 or 18E1. In one embodiment, the antibodies bind to an epitope of ILT2 that at least partially overlaps with, or includes at least one residue in, the epitope bound by antibody 48F12, 3F5, 2H2A, 12D12, 26D8 or 18E1. The residues bound by the antibody can be specified as being present on the surface of the ILT2 polypeptide, e.g., on an ILT2 polypeptide expressed on the surface of a cell.

Binding of anti-ILT2 antibody to a particular site on ILT2 can be assessed by measuring binding of an anti-ILT2 antibody to cells transfected with ILT2 mutants, as compared to the ability of anti-ILT2 antibody to bind wild-type ILT2 polypeptide (e.g., SEQ ID NO: 1). A reduction in binding between an anti-ILT2 antibody and a mutant ILT2 polypeptide (e.g., a mutant of Table 5) means that there is a reduction in binding affinity (e.g., as measured by known methods such FACS testing of cells expressing a particular mutant, or by Biacore testing of binding to mutant polypeptides) and/or a reduction in the total binding capacity of the anti-ILT2 antibody (e.g., as evidenced by a decrease in Bmax in a plot of anti-ILT2 antibody concentration versus polypeptide concentration). A significant reduction in binding indicates that the mutated residue is directly involved in binding to the anti-ILT2 antibody or is in close proximity to the binding protein when the anti-ILT2 antibody is bound to ILT2.

In some embodiments, a significant reduction in binding means that the binding affinity and/or capacity between an anti-ILT2 antibody and a mutant ILT2 polypeptide is reduced by greater than 40%, greater than 50%, greater than 55%, greater than 60%, greater than 65%, greater than 70%, greater than 75%, greater than 80%, greater than 85%, greater than 90% or greater than 95% relative to binding between the antibody and a wild type ILT2 polypeptide. In certain embodiments, binding is reduced below detectable limits. In some embodiments, a significant reduction in binding is evidenced when binding of an anti-ILT2 antibody to a mutant ILT2 polypeptide is less than 50% (e.g., less than 45%, 40%, 35%, 30%, 25%, 20%, 15% or 10%) of the binding observed between the anti-ILT2 antibody and a wild-type ILT2 polypeptide.

In some embodiments, anti-ILT2 antibodies are provided that exhibit significantly lower binding for a mutant ILT2 polypeptide in which a residue in a segment comprising an amino acid residue bound by any of the exemplary antibodies 48F12, 3F5, 2H2A, 12D12, 26D8 or 18E1 is substituted with a different amino acid, compared to a binding to a wild-type ILT2 polypeptide not comprising such substitution(s) (e.g. a polypeptide of SEQ ID NO: 1).

In one aspect, an anti-ILT2 antibody binds an epitope positioned on or within the D1 domain (domain 1) of the human ILT2 protein. In one aspect, an anti-ILT2 antibody competes with antibody 12D12 for binding to an epitope on the D1 domain (domain 1) of the human ILT2 protein.

The D1 domain can be defined as corresponding or having the amino acid sequence as follows:

(SEQ ID NO: 121)
GHLPKPTLWAEPGSVITQGSPVTLRCQGGQETQEYRLYR
EKKTALWITRIPQELVKKGQFPIPSITWEHAGRYRCYYG
SDTAGRSESSDPLELVVTGA

In one aspect, the anti-ILT2 antibody has reduced binding, optionally loss of binding, to an ILT2 polypeptide having a mutation at a residue selected from the group consisting of: E34, R36, Y76, A82 and R84 (with reference to SEQ ID NOS 2 or 121; in bold in SEQ ID NO 55, above); optionally, the mutant ILT2 polypeptide has the mutations: E34A, R36A, Y76I, A82S, R84L. In one embodiment, an antibody furthermore has reduced binding to a mutant ILT2 polypeptide comprising a mutation at one or more (or all of) residues selected from the group consisting of G29, Q30, Q33, T32 and D80 (with reference to SEQ ID NOS: 2 or 121; underlined in SEQ ID NO121, above), optionally, the mutant ILT2 polypeptide has the mutations: G29S, Q30L, Q33A, T32A, D80H. In each case, a decrease or loss of binding can be specified as being relative to binding between the antibody and a wild-type ILT2 polypeptide comprising the amino acid sequence of SEQ ID NO: 2.

In one aspect, the anti-ILT2 antibody binds an epitope on ILT2 comprising an amino acid residue (e.g., one, two, three, four or five of the residues) selected from the group consisting of E34, R36, Y76, A82 and R84 (with reference to SEQ ID NO: 2). In one aspect, the anti-ILT2 antibody binds an epitope on ILT2 comprising an amino acid residue (e.g., one, two, three, four or five of the residues) selected from the group consisting of G29, Q30, Q33, T32 and D80 (with reference to SEQ ID NO: 2). In one aspect, the anti-ILT2 antibody binds an epitope on ILT2 comprising: (i) an amino acid residue (e.g., one, two, three, four or five of the residues) selected from the group consisting of E34, R36, Y76, A82 and R84, and (ii) an amino acid residue (e.g., one, two, three, four or five of the residues) selected from the group consisting of G29, Q30, Q33, T32 and D80.

In one aspect, an anti-ILT2 antibody binds an epitope positioned on or within the D4 domain (domain 4) of the human ILT2 protein. In one aspect the anti-ILT2 antibody binds residues in the segment of residues 1-83 of SEQ ID NO: 122. In one aspect, an anti-ILT2 antibody competes with antibody 26D8 and/or 18E1 for binding to an epitope on the D4 domain (domain 4) of the human ILT2 protein.

The D4 domain can be defined as corresponding or having the amino acid sequence as follows:

(SEQ ID NO: 122)
FYDRVSLSVQPGPTVASGENVTLLCQSQGWMQTFLLTKEG
AADDPWRLRSTYQSQKYQAEFPMGPVTSAHAGTYRCYGSQ
SSKPYLLTHPSDPLELVVSGPSGGPSSPTTGPTSTSGPED
QPLTPTGSDPQSGLGRH

In one aspect, the anti-ILT2 antibody has reduced binding, optionally loss of binding, to an ILT2 polypeptide having a mutation at a residue selected from the group consisting of: F299, Y300, D301, W328, Q378 and K381 (with reference to SEQ ID NO: 2; in bold in SEQ ID NO 122, above); optionally, the mutant ILT2 polypeptide has the mutations: F299I, Y300R, D301A, W328G, Q378A, K381N. In one embodiment, an antibody furthermore has reduced binding to a mutant ILT2 polypeptide comprising a mutation at one or more (or all of) residues selected from the group consisting of W328, Q330, R347, T349, Y350 and Y355 (with reference to SEQ ID NO: 2), optionally, the mutant ILT2 polypeptide has the mutations: W328G, Q330H, R347A, T349A, Y350S, Y355A. In one embodiment, an antibody furthermore has reduced binding to a mutant ILT2 polypeptide comprising a mutation at one or more (or all of) residues selected from the group consisting of D341, D342, W344, R345 and R347 (with reference to SEQ ID NO: 2), optionally, the mutant ILT2 polypeptide has the mutations: D341A, D342S, W344L, R345A, R347A. In each case, a decrease or loss of binding can be specified as being relative to binding between the antibody and a wild-type ILT2 polypeptide comprising the amino acid sequence of SEQ ID NO: 2.

In one aspect, the anti-ILT2 antibody binds an epitope on ILT2 comprising an amino acid residue (e.g., one, two, three, four or five of the residues) selected from the group consisting of F299, Y300, D301, W328, Q378 and K381 (with reference to SEQ ID NO: 2). In one aspect, the anti-ILT2 antibody binds an epitope on ILT2 comprising an amino acid residue (e.g., one, two, three, four or five of the residues) selected from the group consisting of W328, Q330, R347, T349, Y350 and Y355 (with reference to SEQ ID NO: 2). In one aspect, the anti-ILT2 antibody binds an epitope on ILT2 comprising an amino acid residue (e.g., one, two, three, four or five of the residues) selected from the group consisting of D341, D342, W344, R345 and R347 (with reference to SEQ ID NO: 2).

In one aspect, the anti-ILT2 antibody binds an epitope on ILT2 comprising: (i) an amino acid residue (e.g., one, two, three, four or five of the residues) selected from the group consisting of F299, Y300, D301, W328, Q378 and K381, and (ii) an amino acid residue (e.g., one, two, three, four or five of the residues) selected from the group consisting of W328, Q330, R347, T349, Y350 and Y355. In one aspect, the anti-ILT2 antibody binds an epitope on ILT2 comprising: (i) an amino acid residue (e.g., one, two, three, four or five of the residues) selected from the group consisting of F299, Y300, D301, W328, Q378 and K381, (ii) an amino acid residue (e.g., one, two, three, four or five of the residues) selected from the group consisting of W328, Q330, R347, T349, Y350 and Y355, and (iii) an amino acid residue (e.g., one, two, three, four or five of the residues) selected from the group consisting of D341, D342, W344, R345 and R347.

Antibody CDR Sequences Antibodies 48F12, 3F5, 2H2A, 12D12, 26D8 and 18E1 The amino acid sequence of the heavy and light chain variable regions of antibodies 48F12, 3F5, 2H2A, 12D12, 26D8 and 18E1 are listed in Table A, below. In a specific embodiment, provided is an antibody that binds essentially the same epitope or determinant as monoclonal antibody 48F12, 3F5, 2H2A, 12D12, 26D8 or 18E1; optionally the antibody comprises the hypervariable region of antibody 48F12, 3F5, 2H2A, 12D12, 26D8 or 18E1. In any of the embodiments herein, antibody 48F12, 3F5, 2H2A, 12D12, 26D8 or 18E1 can be characterized by the amino acid sequences and/or nucleic acid sequences encoding it. In one embodiment, the monoclonal antibody comprises the Fab or F(ab′)₂ portion of 48F12, 3F5, 2H2A, 12D12, 26D8 or 18E1. Also provided is a monoclonal antibody that comprises the heavy chain variable region of 48F12, 3F5, 2H2A, 12D12, 26D8 or 18E1. According to one embodiment, the monoclonal antibody comprises the three CDRs of the heavy chain variable region of 48F12, 3F5, 2H2A, 12D12, 26D8 or 18E1. Also provided is a monoclonal antibody that further comprises the variable light chain variable region of 48F12, 3F5, 2H2A, 12D12, 26D8 or 18E1 or one, two or three of the CDRs of the light chain variable region of 48F12, 3F5, 2H2A, 12D12, 26D8 or 18E1. The HCDR1, 2, 3 and LCDR1, 2, 3 sequences can optionally be specified as all (or each, independently) being those of the Kabat numbering system, those of the Chotia numbering system, those of the IMGT numbering, or any other suitable numbering system. Optionally any one or more of said light or heavy chain CDRs may contain one, two, three, four or five or more amino acid modifications (e.g. substitutions, insertions or deletions).

In one aspect, provided is an antibody, wherein the antibody comprises: a HCDR1 region of 26D8 comprising an amino acid sequence EHTIH (SEQ ID NO: 14), or a sequence of at least 3, 4 or 5 contiguous amino acids thereof, optionally wherein one or more of these amino acids may be substituted by a different amino acid; a HCDR2 region of 26D8 comprising an amino acid sequence WFYPGSGSMKYNEKFKD (SEQ ID NO: 15), or a sequence of at least 4, 5, 6, 7, 8, 9 or 10 contiguous amino acids thereof, optionally wherein one or more of these amino acids may be substituted by a different amino acid; a HCDR3 region of 26D8 comprising an amino acid sequence HTNWDFDY (SEQ ID NO: 16), or a sequence of at least 4, 5, 6, 7, 8, 9 or 10 contiguous amino acids thereof, optionally wherein one or more of these amino acids may be substituted by a different amino acid; a LCDR1 region of 26D8 comprising an amino acid sequence KASQSVDYGGDSYMN (SEQ ID NO: 17), or a sequence of at least 4, 5, 6, 7, 8, 9 or 10 contiguous amino acids thereof, optionally wherein one or more of these amino acids may be substituted by a different amino acid; a LCDR2 region of 26D8 comprising an amino acid sequence AASNLES (SEQ ID NO: 18), or a sequence of at least 4, 5, or 6 contiguous amino acids thereof, optionally wherein one or more of these amino acids may be substituted by a different amino acid; a LCDR3 region of 26D8 comprising an amino acid sequence QQSNEEPWT (SEQ ID NO: 19), or a sequence of at least 4, 5, 6, 7, or 8 contiguous amino acids thereof, optionally wherein one or more of these amino acids may be deleted or substituted by a different amino acid.

In one aspect, provided is an antibody, wherein the antibody comprises: a HCDR1 region of 18E1 comprising an amino acid sequence AHTIH (SEQ ID NO: 22), or a sequence of at least 3 or 4 contiguous amino acids thereof, optionally wherein one or more of these amino acids may be substituted by a different amino acid; a HCDR2 region of 18E1 comprising an amino acid sequence WLYPGSGSIKYNEKFKD (SEQ ID NO: 23), or a sequence of at least 4, 5, 6, 7, 8, 9 or 10 contiguous amino acids thereof, optionally wherein one or more of these amino acids may be substituted by a different amino acid; a HCDR3 region of 18E1 comprising an amino acid sequence HTNWDFDY (SEQ ID NO: 24), or a sequence of at least 4, 5, 6, 7, 8, 9 or 10 contiguous amino acids thereof, optionally wherein one or more of these amino acids may be substituted by a different amino acid; a LCDR1 region of 18E1 comprising an amino acid sequence KASQSVDYGGASYMN (SEQ ID NO: 25), or a sequence of at least 4, 5, 6, 7, 8, 9 or 10 contiguous amino acids thereof, optionally wherein one or more of these amino acids may be substituted by a different amino acid; a LCDR2 region of 18E1 comprising an amino acid sequence AASNLES (SEQ ID NO: 26), or a sequence of at least 4, 5 or 6 10 contiguous amino acids thereof, optionally wherein one or more of these amino acids may be substituted by a different amino acid; a LCDR3 region of 18E1 comprising an amino acid sequence QQSNEEPWT (SEQ ID NO: 27), or a sequence of at least 4, 5, 6 or 7 contiguous amino acids thereof, optionally wherein one or more of these amino acids may be deleted or substituted by a different amino acid.

In one aspect, provided is an antibody, wherein the antibody comprises: a HCDR1 region of 12D12 comprising an amino acid sequence SYWVH (SEQ ID NO: 30), or a sequence of at least 3 or 4 contiguous amino acids thereof, optionally wherein one or more of these amino acids may be substituted by a different amino acid; a HCDR2 region of 12D12 comprising an amino acid sequence VIDPSDSYTSYNQNFKG (SEQ ID NO: 31), or a sequence of at least 4, 5, 6, 7, 8, 9 or 10 contiguous amino acids thereof, optionally wherein one or more of these amino acids may be substituted by a different amino acid; a HCDR3 region of 12D12 comprising an amino acid sequence GERYDGDYFAMDY (SEQ ID NO: 32), or a sequence of at least 4, 5, 6, 7, 8, 9 or 10 contiguous amino acids thereof, optionally wherein one or more of these amino acids may be substituted by a different amino acid; a LCDR1 region of 12D12 comprising an amino acid sequence RASENIYSNLA (SEQ ID NO: 33), or a sequence of at least 4, 5, 6, 7, 8, 9 or 10 contiguous amino acids thereof, optionally wherein one or more of these amino acids may be substituted by a different amino acid; a LCDR2 region of 12D12 comprising an amino acid sequence AATNLAD (SEQ ID NO: 34), or a sequence of at least 4, 5 or 6 contiguous amino acids thereof, optionally wherein one or more of these amino acids may be substituted by a different amino acid; a LCDR3 region of 12D12 comprising an amino acid sequence QHFWNTPRT (SEQ ID NO: 35), or a sequence of at least 4, 5, 6 or 7 contiguous amino acids thereof, optionally wherein one or more of these amino acids may be deleted or substituted by a different amino acid.

In one aspect, an antibody or antibody fragment comprises: a HCDR1 region of 3F5 comprising an amino acid sequence NYYIQ (SEQ ID NO: 48), or a sequence of at least 3 or 4 contiguous amino acids thereof, optionally wherein one or more of these amino acids may be substituted by a different amino acid; a HCDR2 region of 3F5 comprising an amino acid sequence WIFPGNNETNYNEKFKG (SEQ ID NO: 49), or a sequence of at least 4, 5, 6, 7, 8, 9 or 10 contiguous amino acids thereof, optionally wherein one or more of these amino acids may be substituted by a different amino acid; a HCDR3 region of 3F5 comprising an amino acid sequence SWNYDARWGY (SEQ ID NO: 50), or a sequence of at least 4, 5, 6, 7, 8, 9 or 10 contiguous amino acids thereof, optionally wherein one or more of these amino acids may be substituted by a different amino acid; a LCDR1 region of 3F5 comprising an amino acid sequence RASEIIDSYGISFMH (SEQ ID NO: 51), or a sequence of at least 4, 5, 6, 7, 8, 9 or 10 contiguous amino acids thereof, optionally wherein one or more of these amino acids may be substituted by a different amino acid; a LCDR2 region of 3F5 comprising an amino acid sequence RASNLES (SEQ ID NO: 52), or a sequence of at least 4, 5 or 6 contiguous amino acids thereof, optionally wherein one or more of these amino acids may be substituted by a different amino acid; a LCDR3 region of 3F5 comprising an amino acid sequence QQSNEDPFT (SEQ ID NO: 53), or a sequence of at least 4, 5, 6 or 7 contiguous amino acids thereof, optionally wherein one or more of these amino acids may be deleted or substituted by a different amino acid.

In one aspect, an antibody or antibody fragment comprises: a HCDR1 region of 48F12 comprising an amino acid sequence SYGVS (SEQ ID NO: 54), or a sequence of at least 3 or 4 contiguous amino acids thereof, optionally wherein one or more of these amino acids may be substituted by a different amino acid; a HCDR2 region of 48F12 comprising an amino acid sequence IIWGDGSTNYHSALVS (SEQ ID NO: 55), or a sequence of at least 4, 5, 6, 7, 8, 9 or 10 contiguous amino acids thereof, optionally wherein one or more of these amino acids may be substituted by a different amino acid; a HCDR3 region of 48F12 comprising an amino acid sequence PNWDYYAMDY (SEQ ID NO: 56), or a sequence of at least 4, 5, 6, 7, 8, 9 or 10 contiguous amino acids thereof, optionally wherein one or more of these amino acids may be substituted by a different amino acid; a LCDR1 region of 48F12 comprising an amino acid sequence RASQDISNYLN (SEQ ID NO: 57), or a sequence of at least 4, 5, 6, 7, 8, 9 or 10 contiguous amino acids thereof, optionally wherein one or more of these amino acids may be substituted by a different amino acid; a LCDR2 region of 48F12 comprising an amino acid sequence YTSRLHS (SEQ ID NO: 58), or a sequence of at least 4, 5 or 6 contiguous amino acids thereof, optionally wherein one or more of these amino acids may be substituted by a different amino acid; a LCDR3 region of 48F12 comprising an amino acid sequence QQGITLPLT (SEQ ID NO: 59), or a sequence of at least 4, 5, 6 or 7 contiguous amino acids thereof, optionally wherein one or more of these amino acids may be deleted or substituted by a different amino acid.

In one aspect, an antibody or antibody fragment comprises: a HCDR1 region of 2H2A comprising an amino acid sequence NYYMQ (SEQ ID NO: 60), or a sequence of at least 3 or 4 contiguous amino acids thereof, optionally wherein one or more of these amino acids may be substituted by a different amino acid; a HCDR2 region of 2H2A comprising an amino acid sequence WIFPGSGESNYNEKFKG (SEQ ID NO: 61) or optionally WIFPGSGESSYNEKFKG (SEQ ID NO: 62), or a sequence of at least 4, 5, 6, 7, 8, 9 or 10 contiguous amino acids thereof, optionally wherein one or more of these amino acids may be substituted by a different amino acid; a HCDR3 region of 2H2A comprising an amino acid sequence TWNYDARWGY (SEQ ID NO: 63), or a sequence of at least 4, 5, 6, 7, 8, 9 or 10 contiguous amino acids thereof, optionally wherein one or more of these amino acids may be substituted by a different amino acid; a LCDR1 region of 2H2A comprising an amino acid sequence IPSESIDSYGISFMH (SEQ ID NO: 64), or a sequence of at least 4, 5, 6, 7, 8, 9 or 10 contiguous amino acids thereof, optionally wherein one or more of these amino acids may be substituted by a different amino acid; a LCDR2 region of 2H2A comprising an amino acid sequence RASNLES (SEQ ID NO: 65), or a sequence of at least 4, 5 or 6 contiguous amino acids thereof, optionally wherein one or more of these amino acids may be substituted by a different amino acid; a LCDR3 region of 2H2A comprising an amino acid sequence QQSNEDPFT (SEQ ID NO: 66), or a sequence of at least 4, 5, 6 or 7 contiguous amino acids thereof, optionally wherein one or more of these amino acids may be deleted or substituted by a different amino acid.

The respective VH and VL and antibodies 3H5, 27C10 and 27H5 are shown in SEQ ID NOS: 36-37, 38-39 and 40-41, respectively. The HCDR1, 2, 3 and LCDR1, 2, 3 sequences of the antibodies can optionally be specified as all (or each, independently) being those of the Kabat numbering system, those of the Chotia numbering, those of the IMGT numbering, or any other suitable numbering system. According to one embodiment, an antibody can comprises the HCDR1, 2, 3 and LCDR1, 2, 3 of antibody 3H-5. According to one embodiment, an antibody can comprises the HCDR1, 2, 3 and LCDR1, 2, 3 of antibody 27010. According to one embodiment, an antibody can comprises the HCDR1, 2, 3 and LCDR1, 2, 3 of antibody 27H5.

In another aspect of any of the embodiments herein, any of the CDRs 1, 2 and 3 of the heavy and light chains of 48F12, 3F5, 2H-2A, 12012, 2608, 18E1, 3H-5, 27H-5 or 27010 may be characterized by a sequence of at least 4, 5, 6, 7, 8, 9 or 10 contiguous amino acids thereof, and/or as having an amino acid sequence that shares at least 50%, 60%, 70%, 80%, 85%, 90% or 95% sequence identity with the particular CDR or set of CDRs listed in the corresponding SEQ ID NO.

TABLE A

| Antibody | SEQ ID | |
|---|---|---|
| domain | NO: | Amino Acid Sequence |
| 26D8 VH | 12 | QVQLQQSGAELVKPGASVKLSCKAS |
| | | GYTFTEHTIHWIKQRSGQGLEWIGW |
| | | FYPGSGSMKYNEKFKDKATLTADKS |
| | | SSTVYMELTRLTSEDSAVYFCARHT |
| | | NWDFDYWGQGTTLTVSS |
| 26D8 VL | 13 | DIVLTQSPASLAVSLGQRATISCKA |
| | | SQSVDYGGDSYMNWYQQKPGQPPKL |
| | | LIYAASNLESGIPARFSGSGSGTDL |
| | | TLNIHPVEEDDAAMYYCQQSNEEPW |
| | | TFGGGTKLEIK |
| 18E1 VH | 20 | QVQLQQSGAELVKPGASVRLSCKAS |
| | | GYTFTAHTIHWVKQRSGQGLEWIGW |
| | | LYPGSGSIKYNEKFKDKATLTADKS |
| | | SSTVYMELSRLTSEDSAVYFCARHT |
| | | NWDFDYWGQGTTLTVSS |
| 18E1 VL | 21 | NIVLTQSPASLAVSLGQRATISCKA |
| | | SQSVDYGGASYMNWYQQKPGQPPKL |
| | | LIYAASNLESGIPARFSGSGSGTDL |
| | | TLNIHPVEEEDAAMYYCQQSNEEPW |
| | | TFGGGTKLEIK12D12VH28QVQLQ |
| | | QPGAELVKPGASVRMSCKASGYTFT |
| | | SYWVHWVKQRPGQGLEWIGVIDPSD |
| | | SYTSYNQNFKGKATLTVDTSSKTAY |
| | | IHLSSLTSEDSAVYFCARGERYDGD |
| | | YFAMDYWGQGTSVTVSS |
| 12D12 VL | 29 | DIVMTQSPASLSVSVGETVTITCRA |
| | | SENIYSNLAWYQQKQGKSPQLLVYA |
| | | ATNLADGVPSRFSGSRSGTQYSLKI |
| | | NSLQSEDFGTYYCQHFWNTPRTFGG |
| | | GTKLEIK |
| 3H5 VH | 36 | QVQLKESGPGLVAPSQSLSITCTVS |
| | | GFSLTSYGVSWVRQPPGKGLEWLGV |
| | | IWGDGSTNYHSALISRLSISKDNSK |
| | | SQVFLKLNSLQTDDTATYYCAKPRW |
| | | DDYAMDYWGQGTSVTVSS |
| 3H5 VL | 37 | DIQMTQTTSSLSASLGDRVTISCRA |
| | | SQDISNYLNWYQQKPDGTVKLLIYY |
| | | TSRLHSGVPSRFSGSGSGTDYSLTI |
| | | SNLEQEDIATYFCQQGNTLWTFGGG |
| | | TKLEIK |
| 27C10 VH | 38 | EVQLQESGPGLVKPSQSLSLTCSVT |
| | | GYSITSGYYWNWIRQFPENKLEWMG |
| | | YIRYDGSNNYNPSLNNRISITRDAS |
| | | KNQFFLKLNSVTTEDTATYYCARGW |
| | | LLWFYAVDYWGQGTSVTVSS |
| 27C10 VL | 39 | DVVMTQTPLSLPVSLGDQASISCRS |
| | | SQSIVHTNGNTYLEWYLQKSGQSPK |
| | | LLIYKVSNRLSGVPDRFSGSGSGTD |
| | | FTLKISRVEAEDLGIYYCFQGSHVP |
| | | WTFGGGTKLEIK |
| 27H5 VH | 40 | QVQLKESGPGLVAPSQSLSITCTVS |
| | | GFSLTSYGVSWVRQPPGKGLEWLGV |
| | | IWGDGNTNYHSALISRLSISKDNSK |
| | | SQVFLKLNSLQTDDTATYYCARTNW |
| | | DGWFAYWGQGTLVTVSA |
| 27H5 VL | 41 | DIVMTQSHKFMSTSVGDRVSITCKA |
| | | SQDVGTAVAWYQQKPGQSPKLLIYW |
| | | ASTRHTGVPDRFTGSGSGTDFTLTI |
| | | SNVQSEDLADYFCQQYRSYPLGTFG |
| | | GGTKLEIK |
| 48F12 VH | 42 | DVQLQESGPELVKPGASVKISCKSS |
| | | GYSFTNFYIHWVKQRPGQGLDWIGW |
| | | IFPGTGETNFNEKFKVKAALTADTS |
| | | SSTVYMQLSTLTSEDSAVYFCARSW |
| | | NYDARWGYWGQGTSITVSS |
| 48F12 VL | 43 | DIQMTQTTSSLSASLGDRVTISCRA |
| | | SQDISNYLNWYQQKVDGTVKLLISY |
| | | TSRLHSGVPSRFSGSGSGTDYSLTI |
| | | SNLEQEDIATYFCQQGITLPLTFGA |
| | | GTKLELK |
| 3F5 VH | 44 | QVQLKESGPELVKPGASVKISCKAS |
| | | GYSFTNYYIHWVKQRPGQGLEWIGW |
| | | IFPGSGETNYSEKFKGEAILTADTS |
| | | SNTAYMQLSSLTSEDSAVYFCARSW |
| | | NYDARWGYWGQGTTLTVSS |
| 3F5 VL | 45 | EIVLTQSPASLAVSLGQRATISCRA |
| | | SEIIDSYGISFMHWYQQKPGQPPKL |
| | | LIYRASNLESGIPARFSGSGSRTDF |
| | | TLTINPVEADDVATYYCQQSNEDPF |
| | | TFGSGTKLEIK |
| 2H2A VH | 46 | EVKLEESGPELVKPGASVKLSCKAS |
| | | GYTFTNYYMQWVKQRPGQGLEWIGW |
| | | IFPGSGESSYNEKFKGKATLSADTS |
| | | STTAYMQLSSLTSEDSAVYFCARTW |
| | | NYDARWGYWGQGTTLTVSS |
| 2H2A VL | 47 | DILMTQSPASLAVSLGQRATISCIP |
| | | SESIDSYGISFMHWYQQKPGQPPKL |
| | | LIYRASNLESGIPARFSGSGSRTDF |
| | | TLTINPVEADDVATYYCQQSNEDPF |
| | | TFGSGTKLELK |

Optionally, in any embodiment, an antibody can be specified as having a heavy chain comprising part or all of an antigen binding region of the respective antibody (e.g. heavy chain CDR1, 2 and 3), fused to an immunoglobulin heavy chain constant region of the human IgG type, optionally a human IgG1, IgG2, IgG3 or IgG4 isotype, optionally further comprising an amino acid substitution to reduce effector function (binding to human Fcγ receptors). Optionally, in any embodiment, an 12012, 2608, 18E1 or 27010 antibody can be specified as having a light chain comprising part or all of an antigen binding region of the respective antibody (e.g. light chain CDR1, 2 and 3), fused to an immunoglobulin light chain constant region of the human kappa type.

In any of the antibodies, e.g., 26D8, 18E1 or 27C10, the specified variable region and CDR sequences may comprise sequence modifications, e.g. a substitution (1, 2, 3, 4, 5, 6, 7, 8 or more sequence modifications). In one embodiment, a CDRs 1, 2 and/or 3 of the heavy and light chains comprises one, two, three or more amino acid substitutions, where the residue substituted is a residue present in a sequence of human origin. In one embodiment the substitution is a conservative modification. A conservative sequence modification refers to an amino acid modification that does not significantly affect or alter the binding characteristics of the antibody containing the amino acid sequence. Such conservative modifications include amino acid substitutions, additions and deletions. Modifications can be introduced into an antibody by standard techniques known in the art, such as site-directed mutagenesis and PCR-mediated mutagenesis. Conservative amino acid substitutions are typically those in which an amino acid residue is replaced with an amino acid residue having a side chain with similar physicochemical properties. Specified variable region and CDR sequences may comprise one, two, three, four or more amino acid insertions, deletions or substitutions. Where substitutions are made, preferred substitutions will be conservative modifications. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g. glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g. threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for retained function (i.e., the properties set forth herein) using the assays described herein.

Optionally, in any embodiment herein, an anti-ILT2 antibody can be characterized as being a function-conservative variant of any of the antibodies, heavy and/or light chains, CDRs or variable regions thereof described herein. In one embodiment, the antibody comprises a heavy chain variable region that is a function-conservative variant of the heavy chain variable region of antibody 12D12, 26D8 or 18E1, and a light chain variable region that is a function-conservative variant of the light chain variable region of the respective 12D12, 26D8 or 18E1 antibody. In one embodiment, the antibody comprises a heavy chain that is a function-conservative variant of the heavy chain variable region of antibody 12D12, 26D8 or 18E1 fused to a human heavy chain constant region disclosed herein, optionally a human IgG4 constant region, optionally a constant region of any of SEQ ID NOS: 42-45, and a light chain that is a function-conservative variant of the light chain variable region of the respective 12D12, 26D8 or 18E1 antibody fused to a human Ckappa light chain constant region.

In one embodiment, the anti-ILT2 antibodies can be prepared such that they do not have substantial specific binding to human Fcγ receptors, e.g., any one or more of CD16A, CD16B, CD32A, CD32B and/or CD64). Such antibodies may comprise constant regions of various heavy chains that are known to lack or have low binding to Fcγ receptors. Alternatively, antibody fragments that do not comprise (or comprise portions of) constant regions, such as F(ab′)2 fragments, can be used to avoid Fc receptor binding. Fc receptor binding can be assessed according to methods known in the art, including for example testing binding of an antibody to Fc receptor protein in a BIACORE assay. Also, generally any antibody IgG isotype can be used in which the Fc portion is modified (e.g., by introducing 1, 2, 3, 4, 5 or more amino acid substitutions) to minimize or eliminate binding to Fc receptors (see, e.g., WO 03/101485, the disclosure of which is herein incorporated by reference). Assays such as cell based assays, to assess Fc receptor binding are well known in the art, and are described in, e.g., WO 03/101485.

In one embodiment, the antibody can comprise one or more specific mutations in the Fc region that result in antibodies that have minimal interaction with effector cells. Reduced or abolished effector functions can be obtained by mutation in the Fc region of the antibodies and have been described in the art: N297A mutation, the LALA mutations, (Strohl, W., 2009, Curr. Opin. Biotechnol. vol. 20(6):685-691); and D265A (Baudino et al., 2008, J. Immunol. 181: 6664-69) see also Heusser et al., WO2012/065950, the disclosures of which are incorporated herein by reference. In one embodiment, an antibody comprises one, two, three or more amino acid substitutions in the hinge region. In one embodiment, the antibody is an IgG1 or IgG2 and comprises one, two or three substitutions at residues 233-236, optionally 233-238 (EU numbering). In one embodiment, the antibody is an IgG4 and comprises one, two or three substitutions at residues 327, 330 and/or 331 (EU numbering). Examples of modified Fc IgG1 antibodies that have reduced FcgammaR interaction are the LALA mutant comprising L234A and L235A mutation in the IgG1 Fc amino acid sequence. Another example of an Fc-reduced mutation is a mutation at residue D265, or at D265 and P329 for example as used in an IgG1 antibody as the DAPA (D265A, P329A) mutation (U.S. Pat. No. 6,737,056). Another modified IgG1 antibody comprises a mutation at residue N297 (e.g., N297A, N297S mutation), which results in aglycosylated/non-glycosylated antibodies. Other mutations include: substitutions at residues L234 and G237 (L234A/G237A); substitutions at residues S228, L235 and R409 (S228P/L235E/R409K,T,M,L); substitutions at residues H268, V309, A330 and A331 (H268Q/V309L/A330S/A331S); substitutions at residues C220, C226, C229 and P238 (C220S/C226S/C229S/P238S); substitutions at residues C226, C229, E233, L234 and L235 (C226S/C229S/E233P/L234V/L235A; substitutions at residues K322, L235 and L235 (K322A/L234A/L235A); substitutions at residues L234, L235 and P331 (L234F/L235E/P331S); substitutions at residues 234, 235 and 297; substitutions at residues E318, K320 and K322 (L235E/E318A/K320A/K322A); substitutions at residues (V234A, G237A, P238S); substitutions at residues 243 and 264; substitutions at residues 297 and 299; substitutions such that residues 233, 234, 235, 237, and 238 defined by the EU numbering system, comprise a sequence selected from PAAAP, PAAAS and SAAAS (see WO2011/066501).

In one embodiment, an antibody comprises a heavy chain constant region comprising the amino acid sequence below, or an amino acid sequence at least 90%, 95% or 99% identical thereto but retaining the amino acid residues at Kabat positions 234, 235 and 331 (underlined):

(SEQ ID NO: 123)
A S T K G P S V F P L A P S S K S T
S G G T A A L G C L V K D Y F P E
P V T V S W N S G A L T S G V H T
F P A V L O S S G L Y S L S S V V
T V P S S S L G T Q T Y I C N V N
H K P S N T K V D K R V E P K S C
D K T H T C P P C P A P E A E G G
P S V F L F P P K P K D T L M I S
R T P E V T C V V V D V S H E D P
E V K F N W Y V D G V E V H N A K
T K P R E E Q Y N S T Y R V V S V
L T V L H Q D W L N G K E Y K C K
V S N K A L P A S I E K T I S K A
K G Q P R E P Q V Y T L P P S R E
E M T K N Q V S L T C L V K G F Y
P S D I A V E W E S N G Q P E N N
Y K T T P P V L D S D G S F F L Y
S K L T V D K S R W Q Q G N V F S
C S V M H E A L H N H Y T Q K S L
S L S P G K.

In one embodiment, an antibody comprises a heavy chain constant region comprising the amino acid sequence below, or an amino acid sequence at least 90%, 95% or 99% identical thereto but retaining the amino acid residues at Kabat positions 234, 235 and 331 (underlined):

(SEQ ID NO: 124)
A S T K G P S V F P L A P S S K S T S
G G T A A L G C L V K D Y F P E P V
T V S W N S G A L T S G V H T F P A
V L Q S S G L Y S L S S V V T V P S
S S L G T Q T Y I C N V N H K P S N
T K V D K R V E P K S C D K T H T C
P P C P A P E F E G G P S V F L F P
P K P K D T L M I S R T P E V T C V
V V D V S H E D P E V K F N W Y V D
G V E V H N A K T K P R E E Q Y N S
T Y R V V S V L T V L H Q D W L N G
K E Y K C K V S N K A L P A S I E K
T I S K A K G Q P R E P Q V Y T L P
P S R E E M T K N Q V S L T C L V K
G F Y P S D I A V E W E S N G Q P E
N N Y K T T P P V L D S D G S F F L
Y S K L T V D K S R W Q Q G N V F S
C S V M H E A L H N H Y T Q K S L S
L S P G K.

In one embodiment, an antibody comprises a heavy chain constant region comprising the amino acid sequence below, or an amino acid sequence at least 90%, 95% or 99% identical thereto but retaining the amino acid residues at Kabat positions 234, 235, 237, 330 and 331 (underlined):

(SEQ ID NO: 125)
A S T K G P S V F P L A P S S K S
T S G G T A A L G C L V K D Y F P
E P V T V S W N S G A L T S G V H
T F P A V L Q S S G L Y S L S S V
V T V P S S S L G T Q T Y I C N V
N H K P S N T K V D K R V E P K S
C D K T H T C P P C P A P E A E G
A P S V F L F P P K P K D T L M I
S R T P E V T C V V V D V S H E D
P E V K F N W Y V D G V E V H N A
K T K P R E E Q Y N S T Y R V V S
V L T V L H Q D W L N G K E Y K C
K V S N K A L P S S I E K T I S K
A K G Q P R E P Q V Y T L P P S R
E E M T K N Q V S L T C L V K G F
Y P S D I A V E W E S N G Q P E N
N Y K T T P P V L D S D G S F F L
Y S K L T V D K S R W Q Q G N V F
S C S V M H E A L H N H Y T Q K S
L S L S P G K.

In one embodiment, an antibody comprises a heavy chain constant region comprising the amino acid sequence below, or a sequence at least 90%, 95% or 99% identical thereto but retaining the amino acid residues at Kabat positions 234, 235, 237 and 331 (underlined):

(SEQ ID NO: 126)
A S T K G P S V F P L A P S S K S
T S G G T A A L G C L V K D Y F P
E P V T V S W N S G A L T S G V H
T F P A V L Q S S G L Y S L S S V
V T V P S S S L G T Q T Y I C N V
N H K P S N T K V D K R V E P K S
C D K T H T C P P C P A P E A E G
A P S V F L F P P K P K D T L M I
S R T P E V T C V V V D V S H E D
P E V K F N W Y V D G V E V H N A
K T K P R E E Q Y N S T Y R V V S
V L T V L H Q D W L N G K E Y K C
K V S N K A L P A S I E K T I S K
A K G Q P R E P Q V Y T L P P S R
E E M T K N Q V S L T C L V K G F
Y P S D I A V E W E S N G Q P E N
N Y K T T P P V L D S D G S F F L
Y S K L T V D K S R W Q Q G N V F
S C S V M H E A L H N H Y T Q K S
L S L S P G K.

Fragments and derivatives of antibodies (which are encompassed by the term “antibody” or “antibodies” as used in this application, unless otherwise stated or clearly contradicted by context) can be produced by techniques that are known in the art. “Fragments” comprise a portion of the intact antibody, generally the antigen binding site or variable region. Examples of antibody fragments include Fab, Fab′, Fab′-SH, F (ab′) 2, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific (e.g., bispecific) antibodies formed from antibody fragments. Included, inter alia, are a nanobody, domain antibody, single domain antibody or a “dAb”.

In certain embodiments, the DNA of a hybridoma producing an antibody, can be modified prior to insertion into an expression vector, for example, by substituting the coding sequence for human heavy- and light-chain constant domains in place of the homologous non-human sequences (e.g., Morrison et al., PNAS pp. 6851 (1984)), or by covalently joining to the immunoglobulin coding sequence all or part of the coding sequence for a non-immunoglobulin polypeptide. In that manner, “chimeric” or “hybrid” antibodies are prepared that have the binding specificity of the original antibody. Typically, such non-immunoglobulin polypeptides are substituted for the constant domains of an antibody.

Optionally an antibody is humanized. “Humanized” forms of antibodies are specific chimeric immunoglobulins, immunoglobulin chains or fragments thereof (such as Fv, Fab, Fab′, F (ab′) 2, or other antigen-binding subsequences of antibodies) which contain minimal sequence derived from the murine immunoglobulin. For the most part, humanized antibodies are human immunoglobulins (recipient antibody) in which residues from a complementary-determining region (CDR) of the recipient are replaced by residues from a CDR of the original antibody (donor antibody) while maintaining the desired specificity, affinity, and capacity of the original antibody.

In some instances, Fv framework residues of the human immunoglobulin may be replaced by corresponding non-human residues. Furthermore, humanized antibodies can comprise residues that are not found in either the recipient antibody or in the imported CDR or framework sequences. These modifications are made to further refine and optimize antibody performance. In general, the humanized antibody will comprise substantially all of at least one, and typically two, variable domains, in which all or substantially all of the CDR regions correspond to those of the original antibody and all or substantially all of the FR regions are those of a human immunoglobulin consensus sequence. The humanized antibody optimally also will comprise at least a portion of an immunoglobulin constant region (Fc), typically that of a human immunoglobulin. For further details see Jones et al., Nature, 321, pp. 522 (1986); Reichmann et al, Nature, 332, pp. 323 (1988); Presta, Curr. Op. Struct. Biol., 2, pp. 593 (1992); Verhoeyen et Science, 239, pp. 1534; and U.S. Pat. No. 4,816,567, the entire disclosures of which are herein incorporated by reference.) Methods for humanizing the antibodies are well known in the art.

The choice of human variable domains, both light and heavy, to be used in making the humanized antibodies is very important to reduce antigenicity. According to the so-called “best-fit” method, the sequence of the variable domain of an antibody is screened against the entire library of known human variable-domain sequences. The human sequence which is closest to that of the mouse is then accepted as the human framework (FR) for the humanized antibody (Sims et al., J. Immunol. 151, pp. 2296 (1993); Chothia and Lesk, J. Mol. 196, 1987, pp. 901). Another method uses a particular framework from the consensus sequence of all human antibodies of a particular subgroup of light or heavy chains. The same framework can be used for several different humanized antibodies (Carter et al., PNAS 89, pp. 4285 (1992); Presta et al., J. Immunol., 151, p. 2623 (1993)).

It is further important that antibodies be humanized with retention of high affinity for ILT-2 or NKG2A receptors and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen (s), is achieved. In general, the CDR residues are directly and most substantially involved in influencing antigen binding.

Another method of making “humanized” monoclonal antibodies is to use a XenoMouse (Abgenix, Fremont, CA) as the mouse used for immunization. A XenoMouse is a murine host according that has had its immunoglobulin genes replaced by functional human immunoglobulin genes. Thus, antibodies produced by this mouse or in hybridomas made from the B cells of this mouse, are already humanized. The XenoMouse is described in U.S. Pat. No. 6,162,963, which is herein incorporated in its entirety by reference.

Human antibodies may also be produced according to various other techniques, such as by using, for immunization, other transgenic animals that have been engineered to express a human antibody repertoire (Jakobovitz et al., Nature 362 (1993) 255), or by selection of antibody repertoires using phage display methods. Such techniques are known to the skilled person and can be implemented starting from monoclonal antibodies as disclosed in the present application.

### Compositions and Kits

Also provided herein are pharmaceutical compositions comprising a NKG2A neutralizing agent such as an anti-NKG2A antibody and/or an ILT-2 neutralizing agent such as an anti-ILT-2 antibody. In particular, in one aspect, provided is a pharmaceutical composition containing a neutralizing anti-NKG2A antibody and a neutralizing anti-ILT-2 antibody, and optionally further a pharmaceutically acceptable carrier.

A NKG2A neutralizing antibody and/or an ILT-2-neutralizing antibody can be incorporated in a pharmaceutical formulation in a concentration from 1 mg/ml to 500 mg/ml, wherein said formulation has a pH from 2.0 to 10.0.

The NKG2A neutralizing agent and the anti-ILT-2 agent can be comprised in the same or separate pharmaceutical formulations.

The formulation may further comprise a buffer system, preservative(s), tonicity agent(s), chelating agent(s), stabilizers and surfactants. In one embodiment, the pharmaceutical formulation is an aqueous formulation, i.e., formulation comprising water. Such formulation is typically a solution or a suspension. In a further embodiment, the pharmaceutical formulation is an aqueous solution. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In another embodiment, the pharmaceutical formulation is a freeze-dried formulation, whereto the physician or the patient adds solvents and/or diluents prior to use.

In another embodiment, the pharmaceutical formulation is a dried formulation (e.g. freeze-dried or spray-dried) ready for use without any prior dissolution.

In a further aspect, the pharmaceutical formulation comprises an aqueous solution of such an antibody, and a buffer, wherein the antibody is present in a concentration from 1 mg/ml or above, and wherein said formulation has a pH from about 2.0 to about 10.0.

In a another embodiment, the pH of the formulation is in the range selected from the list consisting of from about 2.0 to about 10.0, about 3.0 to about 9.0, about 4.0 to about 8.5, about 5.0 to about 8.0, and about 5.5 to about 7.5.

In a further embodiment, the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative embodiment of the invention.

In a further embodiment, the formulation further comprises a pharmaceutically acceptable preservative. In a further embodiment, the formulation further comprises an isotonic agent. In a further embodiment, the formulation also comprises a chelating agent. In a further embodiment of the invention the formulation further comprises a stabilizer. In a further embodiment, the formulation further comprises a surfactant. For convenience reference is made to Remington: The Science and Practice of Pharmacy, 19^{th} edition, 1995.

It is possible that other ingredients may be present in the pharmaceutical formulation of the present invention. Such additional ingredients may include wetting agents, emulsifiers, antioxidants, bulking agents, tonicity modifiers, chelating agents, metal ions, oleaginous vehicles, proteins (e.g., human serum albumin, gelatine or proteins) and a zwitterion (e.g., an amino acid such as betaine, taurine, arginine, glycine, lysine and histidine). Such additional ingredients, of course, should not adversely affect the overall stability of the pharmaceutical formulation of the present invention.

Administration of pharmaceutical compositions according to the invention may be through any appropriate route of administration, for example, intravenous. Suitable antibody formulations can also be determined by examining experiences with other already developed therapeutic monoclonal antibodies.

Also provided are kits, for example kits which include:
(i) a pharmaceutical composition containing a NKG2A neutralizing agent such as an anti-NKG2A antibody, and an ILT-2 neutralizing agent such as an anti-ILT-2 antibody, or
(ii) a first pharmaceutical composition containing an ILT-2 neutralizing agent such as an anti-ILT-2 antibody, and a second pharmaceutical composition containing a NKG2A neutralizing agent such as an anti-NKG2A antibody, or
(iii) a pharmaceutical composition containing a NKG2A neutralizing agent such as an anti-NKG2A antibody, and instructions to administer said NKG2A neutralizing agent with an ILT-2 neutralizing agent such as an anti-ILT-2 antibody, or
(iv) a pharmaceutical composition containing an ILT-2 neutralizing agent such as an anti-ILT-2 antibody, and instructions to administer said ILT-2 neutralizing agent antibody with a NKG2A neutralizing agent such as an anti-NKG2A antibody.

A pharmaceutical composition may optionally be specified as comprising a pharmaceutically-acceptable carrier. An anti-NKG2A or anti-ILT-2 antibody may optionally be specified as being present in a therapeutically effective amount adapted for use in any of the methods herein. The kits optionally also can include instructions, e.g., comprising administration schedules, to allow a practitioner (e.g., a physician, nurse, or patient) to administer the composition contained therein to a patient having cancer. In any embodiment, a kit optionally can include instructions to administer said NKG2A neutralizing agent simultaneously, separately, or sequentially with said anti-ILT-2 antibody. In any embodiment, a kit optionally can include instructions for use in the treatment of a cancer (e.g. a cancer further described herein). In any embodiment, a kit optionally can include instructions for use in the treatment of a colorectal cancer, for example. The kit also can include a syringe.

Optionally, the kits include multiple packages of the single-dose pharmaceutical compositions each containing an effective amount of the NKG2A neutralizing agent, and/or the anti-ILT-2 antibody, for a single administration in accordance with the methods provided above. Instruments or devices necessary for administering the pharmaceutical composition(s) also may be included in the kits. For instance, a kit may provide one or more pre-filled syringes containing an amount of the anti-NKG2A or an anti-ILT-2 antibody.

In one embodiment, the present invention provides a kit for treating a cancer or a tumor in a human patient, optionally wherein said cancer or tumor is a HLA-E and/or HLA-G-positive tumor or cancer (and optionally further an PD-L1-negative tumor or cancer), the kit comprising:
(a) a dose of an anti-NKG2A antibody comprising the H-CDR1, H-CDR2 and H-CDR3 domains disclosed herein, optionally the CDRs of a heavy chain variable region having the sequence set forth in any of SEQ ID NOS: 68-72, and the L-CDR1, L-CDR2 and L-CDR3 domains disclosed herein, optionally the CDRs of a light chain variable region having the sequence set forth in SEQ ID NO:73; and/or
(b) a dose of an anti-ILT-2 antibody, optionally wherein the anti-ILT-2 antibody is capable of potentiating the cytotoxicity of primary NK cells, optionally wherein the anti-ILT-2 antibody comprises the H-CDR1, H-CDR2 and H-CDR3 domains of a heavy chain variable region having the sequence set forth in any of SEQ ID NOS: 12, 20, 28, 36, 38, 40, 42, 44, 46, and the L-CDR1, L-CDR2 and L-CDR3 domains of a respective light chain variable region having the sequence set forth in SEQ ID NO: 13, 21, 29, 37, 39, 41, 43, 45, 47; and
(c) optionally, instructions for using said anti-NKG2A antibody and/or said anti-ILT-2 antibody in any of the methods described herein.

### Diagnostics, Prognostics, and Treatment of Malignancies

Described are methods useful in the diagnosis, prognosis, monitoring and treatment of a cancer in an individual. The methods can be for enhancing and/or eliciting an anti-tumor immune response in an individual. The methods can be for enhancing and/or potentiating the activity (e.g. cytotoxic activity toward cancer cells) of NK and/or CD8 T cells (optionally tumor-infiltrating NK and/or CD8 T cells) in an individual. Optionally, the anti-tumor immune response is at least partially mediated by NK and/or CD8 T cells. In another embodiment, the methods can be for enhancing and/or potentiating the anti-tumor immune response mediated by an antibody that neutralizes the inhibitory activity of PD-1. In another embodiment, the methods can be for making an individual having a cancer suitable for treatment with an antibody that neutralizes the inhibitory activity of PD-1. The methods are particularly useful in treatment of colorectal cancer, renal cell carcinoma, lung cancer (e.g. non-small cell lung carcinoma), melanoma, ovarian cancer, endometrial cancer, pancreatic cancer or a head and neck cancer, e.g., head and neck squamous cell carcinoma (HNSCC). Further, as shown herein, in clear cell renal cell carcinoma, elevated ILT-2 expression in tumor samples is associated with decreased survival and yet further head and neck cancer, lung cancer, kidney cancer and ovarian cancer can be associated with elevated expression of ILT-2 on NK cells, and accordingly such cancers can also advantageous be treated in accordance with the methods and compositions of the disclosure.

In some embodiments, provided is a method of treating a tumor in an individual, e.g., renal cell carcinoma (e.g. clear cell renal cell carcinoma), comprising administering to the individual an effective amount of an antibody that neutralizes NKG2A and an antibody that neutralizes the inhibitory activity of ILT-2. In some embodiments, provided is a method of treating a tumor in an individual, e.g., renal cell carcinoma, comprising administering to the individual an effective amount of an antibody that neutralizes NKG2A, an antibody that neutralizes the inhibitory activity of ILT-2, and an antibody that neutralizes the inhibitory activity of PD-1.

In one embodiment, a cancer is known to be characterized by presence of HLA-A expression, HLA-B expression and/or HLA-G-expression, for example as assessed by detecting HLA-A-, HLA-B- and/or HLA-G-expressing cells in the tumor or tumor environment. In one embodiment, the HLA-A-, HLA-B- and/or HLA-G-expressing cells are tumor cells.

In one embodiment, provided is use of an ILT-2-neutralizing antibody in combination with an anti-NKG2A antibody (optionally further in combination with antibody that neutralizes the inhibitory activity of PD-1) as described herein, to advantageously treat a cancer that is HLA-G-positive, optionally that is HLA-G1 and/or HLA-G5 positive. Accordingly, provided is a method for treating or preventing a cancer or tumors in an individual having a HLA-G-positive tumor or cancer, the method comprising administering to the individual an agent that binds and neutralizes NKG2A, e.g., in combination with an antibody that neutralizes the inhibitory activity of ILT-2. In one embodiment, the disclosure provides a method for the treatment or prevention of an HLA-G-positive cancer in an individual, the method comprising: administering to the individual a NKG2A neutralizing agent. In one embodiment, the disclosure provides a method for the treatment or prevention of a HLA-G-positive cancer in an individual, the method comprising: administering to the individual a NKG2A neutralizing agent and an antibody that neutralizes the inhibitor activity of ILT-2. In one embodiment, the HLA-G is HLA-G1. In one embodiment, the HLA-G is HLA-G5.

In one aspect, a HLA-G-positive cancer is of a type or has a profile known to be generally or regularly characterized by presence of HLA-G-expression (e.g. HLA-G-expression at the surface of tumor cells). Accordingly, there is no requirement for a step of testing individuals or biological samples from individuals. In another aspect, HLA-G-expressing cells (e.g. tumor cells) can be detected in the tumor or tumor environment in order to determine if tumor or cancer is HLA-G positive. In one embodiment, the HLA-G-positive cancer is characterized by a tumor determined (e.g. by in vitro detection of HLA-G in a tumor biopsy) to comprise HLA-G-expressing cells. In one embodiment, the HLA-G-positive cancer is characterized by tumor tissue comprising malignant cells that express HLA-G, thus comprising respective HLA-E and/or HLA-G polypeptides. In one embodiment, an HLA-G-positive cancer is characterized by the presence of soluble HLA-G polypeptides, optionally high or increased levels of soluble HLA-G (compared to levels in healthy individuals). Optionally, soluble HLA-G polypeptides are present in circulation.

In another embodiment, provided is a method for determining whether an individual having a cancer, optionally a NSCLC, will derive particular benefit from, be responsive to and/or suitable for treatment with an agent that neutralizes NKG2A and an agent that neutralizes ILT-2, the method comprising determining whether said individual has a HLA-E and/or HLA-G positive cancer, wherein a determination that said individual has a HLA-E and/or HLA-G positive cancer indicates that the individual may derive particularly strong benefit from, be responsive to and/or suitable for treatment with an agent that neutralizes the inhibitory receptor NKG2A and an agent that neutralizes the inhibitory activity of ILT-2 (optionally further in combination with an antibody that neutralizes the inhibitory activity of PD-1).

In some embodiments, provided is a method of treating a tumor in an individual, comprising (i) identifying an individual who has a HLA-G-positive tumor (e.g. a HLA-G1 positive tumor, a HLA-G5 positive tumor), and (ii) administering to the individual a NKG2A-neutralizing agent and antibody that neutralizes the inhibitory activity of ILT-2. In some embodiments, provided is a method of treating a tumor in an individual, comprising (i) identifying an individual who has a HLA-G-positive tumor (e.g. a HLA-G1 positive tumor, a HLA-G5 positive tumor), and (ii) administering to the individual a NKG2A-neutralizing agent, an antibody that neutralizes the inhibitory activity of ILT-2 and an antibody that neutralizes the inhibitory activity of PD-1.

In one embodiment, a HLA-G-positive tumor or cancer is a tumor or cancer known to be generally characterized by presence of HLA-G-expressing cells (HLA-G1 expressing cells) in the tumor or tumor environment or by high levels of soluble HLA-G (e.g., HLA-G5). Accordingly, an individual having a cancer can be treated with the ILT-2 and/or NKG2A neutralizing agent with or without a prior detection step to assess expression of HLA-G on tumor cells.

In one embodiment, the treatment methods can comprise a step of detecting a HLA-G (e.g. HLA-G1 and/or HLA-G5 nucleic acid or polypeptide in a biological sample from an individual. A membrane bound HLA-G polypeptide can for example be detected in a sample of cancer tissue, or tissue proximal to or at the periphery of a cancer, for example cancer adjacent tissue. A soluble HLA-G polypeptide (e.g., HLA-G5) can be detected in a blood-derived sample to assess HLA-G in circulation, or in cancer and/or cancer adjacent tissue. A determination that a biological sample comprises HLA-G polypeptide(s), e.g. comprises cells, optionally cancer cells, that express the HLA-G (e.g. prominently expressing HLA-G; expressing HLA-G at a high level, high intensity of staining with an anti-HLA-G antibody, high levels of soluble HLA-G in circulation, compared to a reference, optionally compared to a healthy individual or healthy tissue reference), indicates that the patient has a cancer that may have a strong benefit from combination treatments of the disclosure. In one embodiment, the method comprises determining the level of expression of a HLA-G nucleic acid or polypeptide in a biological sample and comparing the level to a reference level corresponding to a healthy individual. A determination that a biological sample comprises cells expressing HLA-G nucleic acid or polypeptide at a level that is elevated compared to a reference level indicates that the patient has a cancer that could be treated with any of the combination treatments of the disclosure. In one embodiment, detecting a HLA-G polypeptide in a biological sample comprises detecting HLA-G polypeptide expressed on the surface of a malignant cell. Optionally, detecting a HLA-G polypeptide in a biological sample comprises detecting soluble HLA-G, e.g., in circulation. An HLA-G polypeptide can be specified as being detected in a substantial number of cells taken from a given individual, for example HLA-G polypeptide can be present on at least 5%, 10%, 20% 30%, 40%, 50° %, 60%, 70%, 80%, or more of the tumor cells or the cells in tumor tissue or tumor-adjacent tissue sample (e.g. biopsy) taken from the individual.

Determining whether an individual has a cancer characterized by cells that express a HLA-G polypeptide can for example comprise obtaining a biological sample (e.g. by performing a biopsy) from the individual that comprises cells from the cancer environment (e.g. tumor or tumor adjacent tissue), bringing said cells into contact with an antibody that binds an HLA-G polypeptide, and detecting whether the cells express HLA-G on their surface. Optionally, determining whether an individual has cells that express HLA-G comprises conducting an immunohistochemistry assay.

As used herein, adjunctive or combined administration (co-administration) includes simultaneous administration of the compounds in the same or different dosage form, or separate administration of the compounds (e.g., sequential administration). Thus, a NKG2A-neutralizing agent can be used in combination with the ILT-2 neutralizing antibody. For example, an anti-NKG2A antibody and an anti-ILT2 antibody can be simultaneously administered in a single formulation. Alternatively, the NKG2A-neutralizing agent and anti-ILT-2 antibody can be formulated for separate administration and are administered concurrently or sequentially.

Unless indicated otherwise, any of the treatment regimens and methods described herein may be used with or without a prior step of detecting the expression of HLA molecules on cells in a biological sample obtained from an individual (e.g. a biological sample comprising cancer cells, cancer tissue or cancer-adjacent tissue). In one embodiment, the cancer treated with the methods disclosed herein is a cancer characterized by HLA-E, optionally high levels of HLA-E. In one embodiment, a cancer is a tumor or cancer known to be generally characterized by presence of HLA-E-expressing cells Advantageously, the treatment methods can comprises a step of detecting a HLA-E nucleic acid or polypeptide in a biological sample of a tumor (e.g. on a tumor cell) from an individual. A determination that a biological sample expresses HLA-E (e.g. prominently expresses; expresses HLA-E at a high level, high intensity of staining with an anti-HLA-E antibody, compared to a reference) indicates that the individual has a cancer that may have a strong benefit from treatment with the treatment regimens and methods described herein. In one embodiment, the method comprises determining the level of expression of a HLA-E nucleic acid or polypeptide in a biological sample and comparing the level to a reference level (e.g. a value, weak cell surface staining, etc.). A determination that a biological sample expresses an HLA-E nucleic acid or polypeptide at a level that is increased compared to the reference level may indicate that the individual has a cancer that can be advantageously treated with the treatment regimens and methods described herein. Determining whether an individual has cancer cells that express an HLA-E polypeptide can for example comprise obtaining a biological sample (e.g. by performing a biopsy) from the individual that comprises cancer cells, bringing said cells into contact with an antibody that binds an HLA-E polypeptide, and detecting whether the cells express HLA-E on their surface. Optionally, determining whether an individual has cancer cells that express HLA-E comprises conducting an immunohistochemistry assay. Optionally determining whether an individual has cancer cells that express HLA-E comprises conducting a flow cytometry assay.

In one embodiment, the ILT2-neutralizing antibodies and the NKG2A-neutralizing antibodies lack binding to human CD16A yet potentiate the activity of CD16A-expressing effector cells (e.g., NK or effector T cells). Accordingly, in one embodiment, the treatment regimens and methods described herein that combine ILT2-neutralizing antibodies and the NKG2A-neutralizing antibodies are used in further combination with an Fc domain-containing protein capable of inducing NK cell-mediated ADCC toward a cell to which it is bound, e.g., via CD16A expressed by an NK cell. Typically, such Fc domain-containing protein is an antibody that binds to an antigen of interest, e.g., an antigen present on a tumor cell (tumor antigen) and comprises an Fc domain or portion thereof, and will exhibit binding to the antigen via the antigen binding domain and to Fcγ receptors (e.g., CD16A) via the Fc domain. Tumor antigens are well known in the art, for example Receptor Tyrosine Kinase-like Orphan Receptor 1 (ROR1), B7-H3, B7-H4, B7-H6, Crypto, CD4, CD20, CD30, CD19, CD38, CD47, EGFR, Her2 (ErbB2/Neu), CD22, CD33, CD79, CD123, CD138, CD171, PSCA, PSMA, BCMA, B7H3, CD52, CD56, CD80, CD70, In one embodiment, its ADCC activity will be mediated at least in part by CD16. In one embodiment, the additional therapeutic agent is an antibody having a native or modified human Fc domain, for example an Fc domain from a human IgG1 or IgG3 antibody. Examples of FDA-approved antibodies that induce ADCC include rituximab (for the treatment of lymphomas, CLL), trastuzumab (for the treatment of breast cancer), alemtuzumab (for the treatment of chronic lymphocytic leukemia), daratumumab (for the treatment of multiple myeloma), and cetuximab or panitumumab (for the treatment of colorectal cancer, head and neck squamous cell carcinoma). Examples also include ADCC-enhanced antibodies with modifications to further increase ADCC, such as: GA-101 (hypofucosylated anti-CD20), margetuximab (Fc enhanced anti-HER2), mepolizumab, MEDI-551 (Fc engineered anti-CD19), obinutuzumab (glyco-engineered/hypofucosuylated anti-CD20), ocaratuzumab (Fc engineered anti-CD20), FPA150 (Fc engineered anti-B7H4), XmAb®5574/MOR208 (Fc engineered anti-CD19). In other aspects, a treatment or use may optionally be specified as not being in combination with (or excluding treatment with) an antibody or other agent that binds CD16 and/or is capable of inducing ADCC toward a cell to which it is bound.

In another embodiment, the treatment regimens and methods described herein that combine ILT2-neutralizing antibodies and the NKG2A-neutralizing antibodies can be advantageously used in further combination with an agent that neutralizes the inhibitory activity of human PD-1, e.g., that inhibits the interaction between PD-1 and PD-L1, optionally further in individuals who are (have been determined to be, or are predicted to be) poor responders to (or not sensitive to) treatment with an agent that neutralizes the inhibitory activity of human PD-1. Examples of agents or antibodies that neutralize the inhibitory activity of human PD-1 include antibodies that bind PD1 or PD-L1. Many such antibodies are known and can be used, for example, at the exemplary the doses and/or frequencies that such agents are typically used. In one embodiment, the second or additional second therapeutic agent is an agent (e.g., an antibody) that inhibits the PD-1 axis (i.e. inhibits PD-1 or PD-L1).

PD-1 is an inhibitory member of the CD28 family of receptors that also includes CD28, CTLA-4, ICOS and BTLA. PD-1 is expressed on activated B cells, T cells, and myeloid cells Okazaki et al. (2002) Curr. Opin. Immunol. 14: 391779-82; Bennett et al. (2003) J Immunol 170:711-8). Two ligands for PD-1 have been identified, PD-L1 and PD-L2, that have been shown to downregulate T cell activation upon binding to PD-1 (Freeman et al. (2000) J Exp Med 192:1027-34; Latchman et al. (2001) Nat Immunol 2:261-8; Carter et al. (2002) Eur J Immunol 32:634-43). PD-L1 is abundant in a variety of human cancers (Dong et al. (2002) Nat. Med. 8:787-9). The interaction between PD-1 and PD-L1 results in a decrease in tumor infiltrating lymphocytes, a decrease in T-cell receptor mediated proliferation, and immune evasion by the cancerous cells. Immune suppression can be reversed by inhibiting the local interaction of PD-1 with PD-L1, and the effect is additive when the interaction of PD-1 with PD-L2 is blocked as well. Blockade of PD-1 can advantageously involve use of an antibody that prevents PD-L1-induced PD-1 signaling, e.g. by blocking the interaction with its natural ligand PD-L1. In one aspect the antibody binds PD-1 (an anti-PD-1 antibody); such antibody may block the interaction between PD-1 and PD-L1 and/or between PD-1 and PD-L2. In another aspect the antibody binds PD-L1 (an anti-PD-L1 antibody) and blocks the interaction between PD-1 and PD-L1.

There are currently at least six agents blocking the PD-1/PD-L1 pathway that are marketed or in clinical evaluation, any of these may be useful in combination with the anti-ILT2 antibodies of the disclosure. One agent is BMS-936558 (Nivolumab/ONO-4538, Bristol-Myers Squibb; formerly MDX-1106). Nivolumab, (Trade name Opdivo®) is an FDA-approved fully human IgG4 anti-PD-L1 mAb that inhibits the binding of the PD-L1 ligand to both PD-1 and CD80 and is described as antibody 5C4 in WO 2006/121168, the disclosure of which is incorporated herein by reference. For melanoma patients, the most significant OR was observed at a dose of 3 mg/kg, while for other cancer types it was at 10 mg/kg. Nivolumab is generally dosed at 10 mg/kg every 3 weeks until cancer progression. Another agent is durvalumab (Imfinzi®, MEDI-4736), an anti-PD-L1 developed by AstraZeneca/Medimmune and described in WO2011/066389 and US2013/034559. Another agent is MK-3475 (human IgG4 anti-PD1 mAb from Merck), also referred to as lambrolizumab or pembrolizumab (Trade name Keytruda®) has been approved by the FDA for the treatment of melanoma and is being tested in other cancers. Pembrolizumab was tested at 2 mg/kg or 10 mg/kg every 2 or 3 weeks until disease progression. Another agent is atezolizumab (Tecentriq®, MPDL3280A/RG7446, Roche/Genentech), a human anti-PD-L1 mAb that contains an engineered Fc domain designed to optimize efficacy and safety by minimizing FcγR binding and consequential antibody-dependent cellular cytotoxicity (ADCC). Doses of ≤1, 10, 15, and 25 mg/kg MPDL3280A were administered every 3 weeks for up to 1 year. In phase 3 trial, MPDL3280A is administered at 1200 mg by intravenous infusion every three weeks in NSCLC. In other aspects, a treatment or use may optionally be specified as not being in combination with (or excluding treatment with) an antibody or other agent that inhibits the PD-1 axis.

The treatment regimens and methods described herein can be useful to enhance the activity of an antibody that neutralizes PD-1. For example, certain antibodies that neutralize PD-1 have shown decreased or limited activity in individuals having low or no detectable PD-L1 expression on tumor cells. Accordingly, in one embodiment, an individual treated according to the disclosure can be characterized as having a cancer characterized by low or no (lack of) PD-L1 expressing cancer cells.

In one embodiment, the disclosure provides a method for the treatment or prevention of a cancer (e.g. NSCLC, HNSCC, colorectal cancer (CRC), ovarian cancer, renal cancer) in an individual, the method comprising:
(a) identifying an individual who has a cancer characterized by low or no detectable PD-L1-expressing cancer cells, optionally obtaining a biological sample comprising tumor cells from the individual and quantifying PD-L1-expressing cancer cells, and
(b) upon a determination that the individual has a cancer characterized by low or no detectable PD-L1-expressing cancer cells, administering to the individual an agent that neutralizes the inhibitory activity of ILT-2 in combination with an antibody that neutralizes the inhibitory activity of NKG2A and an antibody neutralizes the inhibitory activity of PD-1.

In certain embodiments, the individual who is treated with the combination of NKG2A-neutralizing agent and PD-1 neutralizing agent has a cancer (e.g., a renal cell cancer, a clear cell renal cell cancer) whose tumor cells express PD-L1.

In other embodiments, the individual who is treated with the combination of NKG2A-neutralizing agent and PD-1 neutralizing agent has a cancer (e.g., a renal cell cancer, a clear cell renal cell cancer) whose tumor cells do not express PD-L1.

It will be appreciated that a treatment method of the disclosure may or may not involve a step of characterizing tumor cell expression of PD-L1 prior to treatment. In one aspect, the invention permits individuals to be treated independently of their PD-L1 status, and accordingly, provided is the use of the combination of NKG2A-neutralizing agent, ILT-2-neutralizing agent and PD-1 neutralizing agent to treat populations of individuals having a cancer, independently or irrespective of tumors PD-L1 expression levels. In one aspect, an individual can be an individual who has not been tested for tumor cell PD-L1 expression.

In some embodiments, the invention includes a method of treating a tumor in an individual who has a cancer, comprising (i) identifying an individual whose tumor cells express PD-L1, and (ii) administering to the individual an effective amount of a NKG2A-neutralizing agent, an effective amount of a ILT2-neutralizing agent and an effective amount of a PD-1 neutralizing agent.

In some embodiments, the invention includes a method of treating a tumor in an individual who has a cancer, comprising (i) identifying an individual whose tumor cells do not express PD-L1, and (ii) administering to the individual an effective amount of a NKG2A-neutralizing agent, an effective amount of a ILT2-neutralizing agent and optionally further an effective amount of a PD-1 neutralizing agent.

In any embodiment herein, a treatment (e.g. treatment with an ILT2-neutralizing agent and a NKG2A-neutralizing agent) can optionally be specified as being in the absence of combined treatment with an agent that binds and/or neutralizes or decreases the inhibitory activity of a (one more more) Killer Ig-like Receptors (KIRs).

The present disclosure also provides an agent that is an antibody that binds to ILT-2 and neutralizes the inhibitory activity of ILT-2 in an NK cell, for use in treating a human individual who has cancer, wherein said antibody that binds ILT-2 is administered in combination with a NKG2A neutralizing agent.

For instance, also provided are:
the agent for use as described above, wherein said individual has a NSCLC, HNSCC, colorectal cancer (CRC), ovarian cancer, renal cancer (e.g. clear cell renal cell carcinoma); the agent for use as described above, wherein said NKG2A neutralizing agent is an antibody that binds a human NKG2A protein, optionally a human or humanized anti-NKG2A antibody;
the agent for use as described above, wherein said NKG2A neutralizing agent is an antibody that inhibits binding of NKG2A to HLA-E;
the agent for use as described above, wherein said NKG2A neutralizing agent comprises the heavy chain H-CDR1, H-CDR2 and H-CDR3 domains having the sequences of SEQ ID NOS: 80-82, and the light chain L-CDR1, L-CDR2 and L-CDR3 domains having the sequences of SEQ ID NOS: 83-85, respectively;
the agent for use as described above, wherein said NKG2A neutralizing agent is monalizumab;
the agent for use as described above, wherein said ILT-2 neutralizing agent is an antibody that binds a human ILT-2 protein, optionally a human or humanized anti-ILT-2 antibody;
the agent for use as described above, wherein said ILT-2-neutralizing agent is an antibody that inhibits binding of ILT-2 to HLA-G1;
the agent for use as described above, wherein said ILT-2-neutralizing agent comprises (a) the heavy chain H-CDR1, H-CDR2 and H-CDR3 domains having the sequences of SEQ ID NOS: 14-16, and the light chain L-CDR1, L-CDR2 and L-CDR3 domains having the sequences of SEQ ID NOS: 17-19, respectively; or (b) the heavy chain H-CDR1, H-CDR2 and H-CDR3 domains having the sequences of SEQ ID NOS: 22-24, and the light chain L-CDR1, L-CDR2 and L-CDR3 domains having the sequences of SEQ ID NOS: 25-27, respectively; (c) the heavy chain H-CDR1, H-CDR2 and H-CDR3 domains having the sequences of SEQ ID NOS: 30-32, and the light chain L-CDR1, L-CDR2 and L-CDR3 domains having the sequences of SEQ ID NOS: 33-35, respectively; (d) the heavy chain H-CDR1, H-CDR2 and H-CDR3 domains having the sequences of SEQ ID NOS: 48-50, and the light chain L-CDR1, L-CDR2 and L-CDR3 domains having the sequences of SEQ ID NOS: 51-53, respectively; (e) the heavy chain H-CDR1, H-CDR2 and H-CDR3 domains having the sequences of SEQ ID NOS: 54-56, and the light chain L-CDR1, L-CDR2 and L-CDR3 domains having the sequences of SEQ ID NOS: 57-59, respectively; or (f) the heavy chain H-CDR1, H-CDR2 and H-CDR3 domains having the sequences of SEQ ID NOS: 60, 61 (or 62 for H-CDR2), and 63, and the light chain L-CDR1, L-CDR2 and L-CDR3 domains having the sequences of SEQ ID NOS: 64-66, respectively;
the agent for use as described above, wherein said NKG2A neutralizing agent and said antibody that binds ILT-2 are administered simultaneously, separately, or sequentially;
the agent for use as described above, wherein said NKG2A neutralizing agent and said antibody that binds ILT-2 are formulated for separate administration and are administered concurrently or sequentially; and/or
the agent for use as described above, wherein said NKG2A neutralizing agent is administered at a dose ranging from 0.1 to 10 mg/kg and said antibody that binds ILT-2 is administered at a dose ranging from 1 to 20 mg/kg. In one embodiment, an ILT-2-neutralizing antibody can be administered in an amount that induces or increases immune cell (e.g. CD8 T cell, NK cell) infiltration into a tumor.

In the combination treatment methods, when NKG2A-neutralizing agent is administered in combination with an ILT-2-neutralizing antibody, the NKG2A-neutralizing agent and ILT-2-neutralizing antibody can be administered separately, together or sequentially, or in a cocktail. In some embodiments, the NKG2A-neutralizing agent is administered prior to the administration of the ILT-2-neutralizing antibody. For example, the NKG2A-neutralizing agent can be administered approximately 0 to 30 days prior to the administration of the ILT-2-neutralizing antibody. In some embodiments, antibody NKG2A-neutralizing agent is administered from about 30 minutes to about 2 weeks, from about 30 minutes to about 1 week, from about 1 hour to about 2 hours, from about 2 hours to about 4 hours, from about 4 hours to about 6 hours, from about 6 hours to about 8 hours, from about 8 hours to 1 day, or from about 1 to 5 days prior to the administration of the anti-ILT-2 antibodies. In some embodiments, a NKG2A-neutralizing agent is administered concurrently with the administration of the ILT-2-neutralizing antibody. In some embodiments, a NKG2A-neutralizing agent is administered after the administration of the ILT-2-neutralizing antibody. For example, a NKG2A-neutralizing agent can be administered approximately 0 to 30 days after the administration of the ILT-2-neutralizing antibody. In some embodiments, a NKG2A-neutralizing agent is administered from about 30 minutes to about 2 weeks, from about 30 minutes to about 1 week, from about 1 hour to about 2 hours, from about 2 hours to about 4 hours, from about 4 hours to about 6 hours, from about 6 hours to about 8 hours, from about 8 hours to 1 day, or from about 1 to 5 days after the administration of the ILT-2-neutralizing antibody.

### EXAMPLES

### Example 1: ILT2 (LILRB1) is Expressed on Healthy Human Donor Memory CD8 T Cells and CD56dim NK Cells

LILRB1 expression on peripheral blood mononuclear cells was determined by flow cytometry on fresh whole blood from healthy human donors. The NK population was determined as CD3-CD56+ cells (anti CD3 AF700—BioLegend #300424; anti CD56 BV421—BD Biosciences #740076). Among NK cells, CD56bright subset was identify as CD16− cells whereas CD56dim subset as CD16+ cells (anti CD16 BV650—BD Biosciences #563691). CD4+ and CD8+ T cells were identify as CD3+CD56-CD4+ and CD3+CD56-CD8+ cells, respectively (CD3—see above; CD4 BV510—BD Biosciences #740161; CD8 BUV737—BD Biosciences #564629). Among the CD4+ T cell population, Tconv and Treg were identify as CD127+CD25−/low and CD127lowCD25high cells, respectively (CD127 PE-Cy7—BD Biosciences #560822; CD25 VioBright—Miltenyi Biotec #130-104-274). Among the CD8+ T cell population, the naïve, central memory, effector memory and effector memory T cell populations were identify as CD45RA+CCR7+, CD45RA-CCR7+, CD45RA-CCR7−, CD45RA+CCR7− cells, respectively (CD45RA BUV395—BD Biosciences #740298; CCR7 PerCP-Cy5.5—BioLegend #353220). A population named “CD3+CD56+ ly” was an heterogeneous cell population comprising NKT cells and γδ T cells. Monocytes were identify as CD3−CD56−CD14+ cells (CD14 BV786—BD Biosciences #563691) and B cells as CD3−CD56−CD19+ cells (CD19 BUV496—BD Biosciences #564655). Anti-LILRB1 antibody (clone HP-F1-APC—BioLegend #17-5129-42) as used. Whole blood was incubated 20 min at RT in the dark with staining Ab mix then red blood cells were lyzed with Optilyse C (Beckman Coulter #A11895) following the provider TDS. Cells were washed twice with PBS and fluorescence was revealed with Fortessa flow cytometer (BD Biosciences).

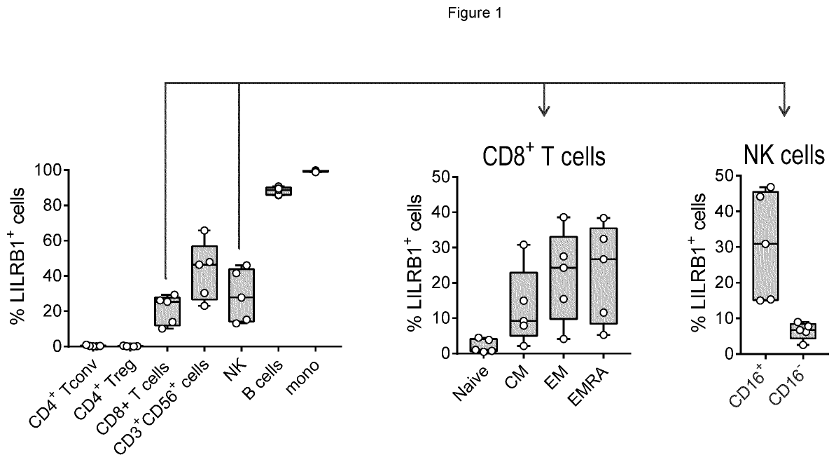

Results are shown in FIG. 1. While B lymphocytes and monocytes generally always express ILT2, conventional CD4 T cells and CD4 Treg cells did not express ILT2, but a significant fraction of CD8 T cells (about 25%), CD3+CD56+ lymphocytes (about 50%) and NK cells (about 30%) expressed ILT2, suggesting that a proportion of each of such CD8 T and NK cell populations can be inhibited by ILT2, as a function of the HLA class I ligands present, for example on tumor cells.

Among the CD8 T cells, ILT2 expression was not present on naïve cells, but was present in effector memory fraction of CD8 T cells, and to a lesser extent, central memory CD8 T cells. Among the NK cells, the ILT2 expression was essentially only on the CD16+ subset (CD56dim), and much less frequently on CD16− NK cells (CD56bright).

### Example 2: ILT2 is Upregulated in Multiple Human Cancers

ILT2 expression on monocytes, B cells, CD4+ T cells, CD8+ T cells and both CD16− and CD16+NK cells was determined by flow cytometry on peripheral blood mononuclear cells (PBMC) purified from whole blood of human cancer patient donors. Cell populations were identified and ILT2 expression was assess using the same antibody mix detailed in example 1. PBMC were incubated 20 min at 4° C. in the dark with the antibody mix, wash twice in staining buffer and fluorescence was measured on a Fortessa flow cytometer.

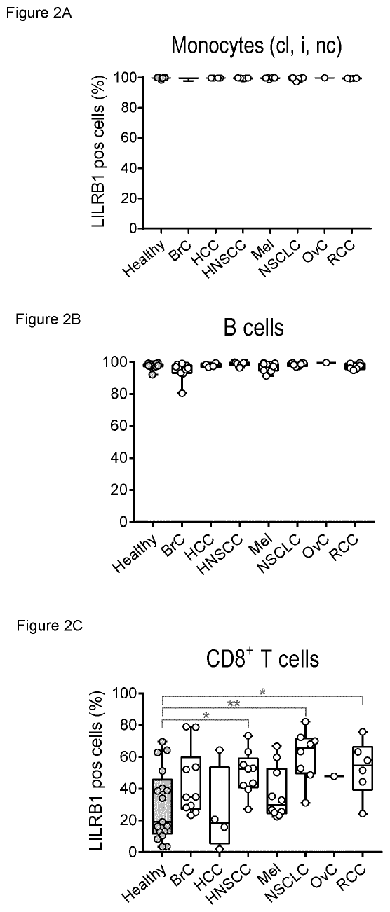

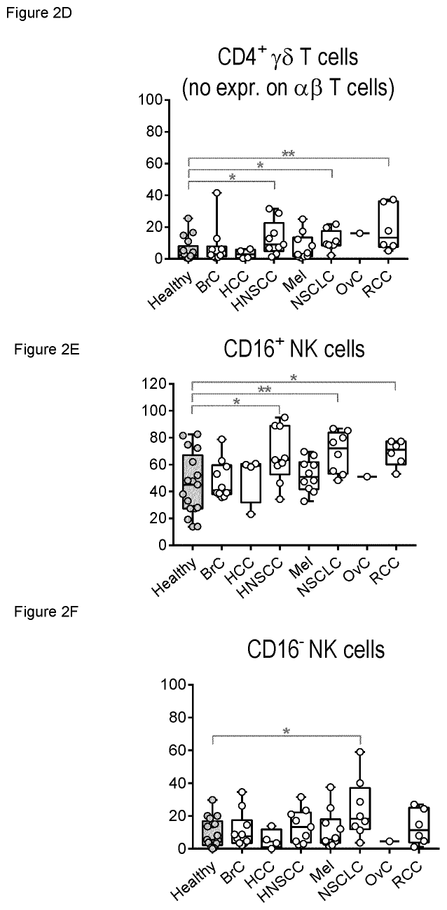

Results from the cancer patient samples are shown in FIG. 2. As can be seen, ILT2 was once again expressed on all monocytes and B cells. However on the lymphocyte subsets, NK cells and CD8 T cells, ILT2 was expressed more frequently with statistical significance on cells from three types of cancers, HNSCC, NSCLC and RCC. ILT2 was upregulated also in ovarian cancer although greater numbers of patient samples need to be studied. This increased expression of ILT2 in cancer patient samples was observed in CD8 T cells, γδ T cells (no expression on as T cells) and CD16+NK cells, in head and neck cancer (HNSCC), lung cancer (NSCLC) and kidney cancer (RCC).

### Example 3: Generation of Anti-ILT2 Antibodies

### Materials and Methods

### Cloning and Production of the ILT-2_6×His Recombinant Protein

The ILT-2 protein (Uniprot access number Q8NHL6) was cloned into the pTT-5 vector between the NruI and BamHI restriction sites. A heavy chain peptide leader was used. The PCR were performed with the following primers:

ILT-2_For_
(SEQ ID NO: 127)
ACAGGCGTGCATTCGGGGCACCTCCCCAAGCCCAC,
ILT-2_Rev_
(SEQ ID NO: 128)
CGAGGTCGGGGGATCCTCAATGGTGGTGATGATG
GTGGTGCCTTCCCAGACCACTCTG,

A 6×His tag was added at the C-terminal part of the protein for purification. The EXP1293 cell line was transfected with the generated vector for transient production. The protein was purified from the supernatant using Ni-NTA beads and monomers were purified using a SEC.

The amino acid sequence for the ILT-2_6×His recombinant protein is shown below:

(SEQ ID NO: 129)
GHLPKPTLWAEPGSVITQGSPVTLRCQGGQETQEYRLYREKKTALW
ITRIPQELVKKGQFPIPSITWEHAGRYRCYYGSDTAGRSESSDPL
ELVVTGAYIKPTLSAQPSPVVNSGGNVILQCDSQVAFDGFSLCKE
GEDEHPQCLNSQPHARGSSRAIFSVGPVSPSRRWWYRCYAYDSNS
PYEWSLPSDLLELLVLGVSKKPSLSVQPGPIVAPEETLTLQCGSD
AGYNRFVLYKDGERDFLQLAGAQPQAGLSQANFTLGPVSRSYGGQ
YRCYGAHNLSSEWSAPSDPLDILIAGQFYDRVSLSVQPGPTVASG
ENVTLLCQSQGWMQTELLTKEGAADDPWRLRSTYQSQKYQAEFPM
GPVTSAHAGTYRCYGSQSSKPYLLTHPSDPLELVVSGPSGGPSSP
TTGPTSTSGPEDQPLTPTGSDPQSGLGRHHHHHHH

### Generation of CHO and KHYG Cell Lines Expressing ILT Family Members at the Cell Surface

The complete forms of ILT-2 were amplified by PCR using the following primers: ILT-2_For ACAGGCGTGCATTCGGGGCACCTCCCCAAGCCC (SEQ ID NO: 130), and ILT-2_Rev_CCGCCCCGACTCTAGACTAGTGGATGGCCAGAGTGG (SEQ ID NO: 131). The PCR products were inserted into the expression vector at appropriate restriction sites. A heavy chain peptide leader was used. The vectors were then transfected into the CHO and KHYG cell lines to obtain stable clones expressing the ILT-2 protein at the cell surface. These cells were then used for hybridoma screening. CHO cells expressing other ILT family members were prepared similarly, including cells expressing ILT-1, ILT-3, ILT-4, ILT-5, ILT-6, ILT7 and ILT-8. The amino acid sequences of the ILT proteins used to prepare the ILT-1, ILT-3, ILT-4, ILT-5 and ILT-6-expressing cells are provided in Table 1 below.

TABLE 1
ILT sequences

| | SEQ ID | |
|---|---|---|
| PrQtein | NO | Sequence (AA) |
| Human | 3 | GHLPKPTLWAEPGSVIIQGSPVTLRCQGSLQAEEYHLYRENKSASWVRRIQEP |
| ILT-1 | | GKNGQFPIPSITWEHAGRYHCQYYSHNHSSEYSDPLELVVTGAYSKPTLSALP |
| | | SPVVTLGGNVTLQCVSQVAFDGFILCKEGEDEHPQRLNSHSHARGWSWAIFSV |
| | | GPVSPSRRWSYRCYAYDSNSPYVWSLPSDLLELLVPGVSKKPSLSVQPGPMVA |
| | | PGESLTLQCVSDVGYDRFVLYKEGERDFLQRPGWQPQAGLSQANFTLGPVSPS |
| | | HGGQYRCYSAHNLSSEWSAPSDPLDILITGQFYDRPSLSVQPVPTVAPGKNVT |
| | | LLCQSRGQFHTFLLTKEGAGHPPLHLRSEHQAQQNQAEFRMGPVTSAHVGTYR |
| | | CYSSLSSNPYLLSLPSDPLELVVSASLGQHPQDYTVENLIRMGVAGLVLVVLG |
| | | ILLFEAQHSQR |
| Human | 2 | GHLPKPTLWAEPGSVITQGSPVTLRCQGGQETQEYRLYREKKTAPWITRIPQE |
| ILT-2 | | LVKKGQFPIPSITWEHAGRYRCYYGSDTAGRSESSDPLELVVTGAYIKPTLSA |
| | | QPSPVVNSGGNVTLQCDSQVAFDGFILCKEGEDEHPQCLNSQPHARGSSRAIF |
| | | SVGPVSPSRRWWYRCYAYDSNSPYEWSLPSDLLELLVLGVSKKPSLSVQPGPI |
| | | VAPEETLTLQCGSDAGYNRFVLYKDGERDFLQLAGAQPQAGLSQANFTLGPVS |
| | | RSYGGQYRCYGAHNLSSEWSAPSDPLDILIAGQFYDRVSLSVQPGPTVASGEN |
| | | VTLLCQSQGWMQTFLLTKEGAADDPWRLRSTYQSQKYQAEFPMGPVTSAHAGT |
| | | YRCYGSQSSKPYLLTHPSDPLELVVSGPSGGPSSPTTGPTSTSAGPEDQPLTP |
| | | TGSDPQSGLGRHLGVVIGILVAVILLLLLLLLLFLILRHRRQGKHWTSTQRKA |
| | | DFQHPAGAVGPEPTDRGLQWRSSPAADAQEENLYAAVKHTQPEDGVEMDTRSP |
| | | HDEDPQAVTYAEVKHSRPRREMASPPSPLSGEFLDTKDRQAEEDRQMDTEAAA |
| | | SEAPQDVTYAQLHSLTLRRKATEPPPSQEGEPPAEPSIYATLAIH |
| Human | 4 | GPLPKPTLWAEPGSVISWGNSVTIWCQGTLEAREYRLDKEESPAPWDRQNPLE |
| ILT-3 | | PKNKARFSIPSMTEDYAGRYRCYYRSPVGWSQPSDPLELVMTGAYSKPTLSAL |
| | | PSPLVTSGKSVTLLCQSRSPMDTFLLIKERAAHPLLHLRSEHGAQQHQAEFPM |
| | | SPVTSVHGGTYRCFSSHGFSHYLLSHPSDPLELIVSGSLEGPRPSPTRSVSTA |
| | | GPEDQPLMPTGSVPHSGLRRHWEVLIGVLVVSILLLSLLLFLLLQHWRQGKHR |
| | | TLAQRQADFQRPPGAAEPEPKDGGLQRRSSPAADVQGENFCAAVKNTQPEDGV |
| | | EMDTRQSPHDEDPQAVTYAKVKHSRPRREMASPPSPLSGEFLDTKDRQAEEDR |
| | | QMDTEAAASEAPQDVTYAQLHSFTLRQKATEPPPSQEGASPAEPSVYA |
| Human | 5 | GTIPKPTLWAEPDSVITQGSPVTLSCQGSLEAQEYRLYREKKSASWITRIRPE |
| ILT-4 | | LVKNGQFHIPSITWEHTGRYGCQYYSRARWSELSDPLVLVMTGAYPKPTLSAQ |
| | | PSPVVTSGGRVTLQCESQVAFGGFILCKEGEEEHPQCLNSQPHARGSSRAIFS |
| | | VGPVSPNRRWSHRCYGYDLNSPYVWSSPSDLLELLVPGVSKKPSLSVQPGPVV |
| | | APGESLTLQCVSDVGYDREVLYKEGERDLRQLPGRQPQAGLSQANFTLGPVSR |
| | | SYGGQYRCYGAHNLSSECSAPSDPLDILITGQIRGTPFISVQPGPTVASGENV |
| | | TLLCQSWRQFHTFLLTKAGAADAPLRLRSIHEYPKYQAEFPMSPVTSAHAGTY |
| | | RCYGSLNSDPYLLSHPSEPLELVVSGPSMGSSPPPTGPISTPGPEDQPLTPTG |
| | | SDPQSGLGRHLGVVIGILVAVVLLLLLLLLLFLILRHRRQGKHWTSTQRKADE |
| | | QHPAGAVGPEPTDRGLQWRSSPAADAQEENLYAAVKDTQPEDGVEMDTRAAAS |
| | | EAPQDVTYAQLHSLTLRRKATEPPPSQEREPPAEPSIYATLAIH |
| Human | 6 | GPFPKPTLWAEPGSVISWGSPVTIWCQGSLEAQEYRLDKEGSPEPLDRNNPLE |
| ILT-5 | | PKNKARFSIPSMTEHHAGRYRCHYYSSAGWSEPSDPLELVMTGFYNKPTLSAL |
| | | PSPVVASGGNMTLRCGSQKGYHHFVLMKEGEHQLPRTLDSQQLHSGGFQALFP |
| | | VGPVNPSHRWRFTCYYYYMNTPQVWSHPSDPLEILPSGVSRKPSLLTLQGPVL |
| | | APGQSLTLQCGSDVGYDRFVLYKEGERDFLQRPGQQPQAGLSQANFTLGPVSP |
| | | SHGGQYRCYGAHNLSSEWSAPSDPLNILMAGQIYDTVSLSAQPGPTVASGENV |
| | | TLLCQSWWQFDTFLLTKEGAAHPPLRLRSMYGAHKYQAEFPMSPVTSAHAGTY |
| | | RCYGSYSSNPHLLSHPSEPLELVVSGHSGGSSLPPTGPPSTPGLGRYLEVLIG |
| | | VSVAFVLLLFLLLFLLLRRQRHSKHRTSDQRKTDFQRPAGAAETEPKDRGLLR |
| | | RSSPAADVQEENLYAAVKDTQSEDRVELDSQSPHDEDPQAVTYAPVKHSSPRR |
| | | EMASPPSSLSGEFLDTKDRQVEEDRQMDTEAAASEASQDVTYAQLHSLTLRRK |
| | | ATEPPPSQEGEPPAEPSIYA |
| Human | 7 | GPLPKPTLWAEPGSVITQGSPVTLRCQGSLETQEYHLYREKKTALWITRIPQE |
| ILT-6 | | LVKKGQFPILSITWEHAGRYCCIYGSHTAGLSESSDPLELVVTGAYSKPTLSA |
| | | LPSPVVTSGGNVTIQCDSQVAFDGFILCKEGEDEHPQCLNSHSHARGSSRAIF |
| | | SVGPVSPSRRWSYRCYGYDSRAPYVWSLPSDLLGLLVPGVSKKPSLSVQPGPV |
| | | VAPGEKLTFQCGSDAGYDRFVLYKEWGRDFLQRPGRQPQAGLSQANFTLGPVS |
| | | RSYGGQYTCSGAYNLSSEWSAPSDPLDILITGQIRARPFLSVRPGPTVASGEN |
| | | VTLLCQSQGGMHTFLLTKEGAADSPLRLKSKRQSHKYQAEFPMSPVTSAHAGT |
| | | YRCYGSLSSNPYLLTHPSDPLELVVSGAAETLSPPQNKSD |
| Human | 8 | ENLPKPILWAEPGPVITWHNPVTIWCQGTLEAQGYRLDKEGNSMSRHILKTLE |
| ILT-7 | | SENKVKLSIPSMMWEHAGRYHCYYQSPAGWSEPSDPLELVVTAYSRPTLSALP |
| | | SPVVTSGVNVTLRCASRLGLGRFTLIEEGDHRLSWTLNSHQHNHGKFQALFPM |
| | | GPLTFSNRGTFRCYGYENNTPYVWSEPSDPLQLLVSGVSRKPSLLTLQGPVVT |
| | | PGENLTLQCGSDVGYIRYTLYKEGADGLPQRPGRQPQAGLSQANFTLSPVSRS |
| | | YGGQYRCYGAHNVSSEWSAPSDPLDILIAGQISDRPSLSVQPGPTVTSGEKVT |
| | | LLCQSWDPMFTFLLTKEGAAHPPLRLRSMYGAHKYQAEFPMSPVTSAHAGTYR |
| | | CYGSRSSNPYLLSHPSEPLELVVSGATETLNPAQKKSDSKTAPHLQDYTVENL |
| | | IRMGVAGLVLLFLGILLFEAQHSQRSPPRCSQEANSRKDNAPFRVVE |
| Human | 9 | GPFPKPTLWAEPGSVISWGSPVTIWCQGSLEAQEYQLDKEGSPEPLDRNNPLE |
| ILT-8 | | PKNKARFSIPSMTQHHAGRYRCHYYSSAGWSEPSDPLELVMTGFYNKPTLSAL |
| | | PSPVVASGGNMTLRCGSQKGYHHFVLMKEGEHQLPRTLDSQQLHSGGFQALFP |
| | | VGPVTPSHRWRFTCYYYYTNTPRVWSHPSDPLEILPSGVSRKPSLLTLQGPVL |
| | | APGQSLTLQCGSDVGYDRFVLYKEGERDFLQRPGQQPQAGLSQANFTLGPVSP |
| | | SHGGQYRCYGAHNLSSEWSAPSDPLNILMAGQIYDTVSLSAQPGPTVASGENV |
| | | TLLCQSRGYFDTFLLTKEGAAHPPLRLRSMYGAHKYQAEFPMSPVTSAHAGTY |
| | | RCYGSYSSNPHLLSFPSEPLELMVSASHAKDYTVENLIRMGMAGLVLVELGIL |
| | | LFEAQHSQRNPQD |

### Generation of K562 Cell Line Expressing HLA-G at the Cell Surface

The complete forms of HLA-G (Genbank access number NP_002118.1, sequence shown below) was amplified by POR using the following primers: HLA-G_For 5′ CCAGAACACAGGATCCGCCGCCACCATGGTGGTCATGGCGCCC 3′ (SEQ ID NO: 132), HLA-G_Rev_5′ TTTTCTAGGTCTCGAGTCAATCTGAGCTCTTCTTTC 3′ (SEQ ID NO: 133). The PCR products were inserted into a vector between the BamHI and XhoI restriction sites and used to transduce K562 cell lines which either did not express HLA-E or were engineered to stably overexpress HLA-E.

```
HLA-G amino acid sequence:
(SEQ ID NO: 10)
    1 MVVMAPRTLF LLLSGALTLT ETWAGSHSMR YFSAAVSRPG RGEPRFIAMG YVDDTQFVRF
  61 DSDSACPRME PRAPWVEQEG PEYWEEETRN TKAHAQTDRM NLQTLRGYYN QSEASSHTLQ
121 WMIGCDLGSD GRLLRGYEQY AYDGKDYLAL NEDLRSWTAA DTAAQISKRK CEAANVAEQR
181 RAYLEGTCVE WLHRYLENGK EMLQRADPPK THVTHHPVED YEATLRCWAL GFYPAEIILT
241 WQRDGEDQTQ DVELVETRPA GDGTFQKWAA VVVPSGEEQR YTCHVQHEGL PEPLMLRWKQ
301 SSLPTIPIMG IVAGLVVLAA VVTGAAVAAV LWRKKSSD.
HLA-E amino acid sequence (Uniprot P 13747):
(SEQ ID NO: 11)
                10 20 30 40 50
MVDGTLLLLL SEALALTQTW AGSHSLKYFH TSVSRPGRGE PRFISVGYVD
                60 70 80 90 100
DTQFVREDND AASPRMVPRA PWMEQEGSEY WDRETRSARD TAQIFRVNLR
              110 120 130 140 150
TLRGYYNQSE AGSHTLQWMH GCELGPDGRF LRGYEQFAYD GKDYLTLNED
              160 170 180 190 200
LRSWTAVDTA AQISEQKSND ASEAEHQRAY LEDTCVEWLH KYLEKGKETL
              210 220 230 240 250
LHLEPPKTHV THHPISDHEA TLRCWALGFY PAEITLTWQQ DGEGHTQDTE
              260 270 280 290 300
LVETRPAGDG TFQKWAAVVV PSGEEQRYTC HVQHEGLPEP VTLRWKPASQ
              310 320 330 340 350
PTIPIVGIIA GLVLLGSVVS GAVVAAVIWR KKSSGGKGGS YSKAEWSDSA
QGSESHSL.
```

### Immunization and Screening

An immunization was performed by immunizing balb/c mice with ILT-2_6×His protein. After the immunization protocol the mice were sacrificed to perform fusions and get hybridomas. The hybridoma supernatants were used to stain CHO-ILT2 and CHO-ILT4 cell lines to check for monoclonal antibody reactivities in a flow cytometry experiment. Briefly, the cells were incubated with 50 μl of supernatant for 1H at 4° C., washed three times and a secondary antibody Goat anti-mouse IgG Fc specific antibody coupled to AF647 was used (Jackson Immunoresearch, J1115-606-071). After 30 min of staining, the cells were washed three times and analyzed using a FACS CANTO II (Becton Dickinson).

About 1500 hybridoma supernatants were screened, to identify those producing antibodies that bind to ILT2 and have the ability to block the interaction between ILT2 with HLA-G. Briefly, recombinant 6×HIS tagged ILT2 was incubated with 50 μl of hybridoma supernatant for 20 min at RT prior incubation with 10⁵ K562 cells expressing HLA-G. Then, cells were washed once and incubated with a secondary complex made of rabbit anti-6×HIS (Bethyl lab, A190-214A) antibody and anti-rabbit IgG F(ab′)² antibody coupled to PE (Jackson lab, 111-116-114). After 30 min of staining, the cells were washed once in PBS and fixed with Cell Fix (Becton Dickinson, 340181). Analysis was performed on a FACS CANTO II flow cytometer.

This assays permitted the identification of a panel of anti-ILT2 antibodies that were highly effective in blocking the interaction of ILT2 with its HLA class I ligand HLA-G. Antibodies 3H5, 12D12, 26D8, 18E1, 27C10, 27H5, 1C11, 1D6, 9G1, 19F10a and 27G10 were identified as having good blocking activity and thus selected for further study.

The resulting antibodies were produced as modified human IgG1 antibodies having heavy chains with Fc domain mutations L234A/L235E/G237A/A330S/P331 S (Kabat EU numbering) which resulted in lack of N-linked glycosylation and substantially lack of binding to human Fcγ receptors CD16A, CD16B, CD32A, CD32B and CD64, Briefly, the VH and Vk sequences of each antibody (the VH and Vk variable regions shown in herein) were cloned into expression vectors containing the huIgG1 constant domains harboring the aforementioned mutations and the huCk constant domain respectively. The two obtained vectors were co-transfected into the CHO cell line. The established pool of cell was used to produce the antibody in the CHO medium.

### Example 4: Binding of Modified Human IgG1 Fc Domains to FcγR

The L234A/L235E/G237A/A330S/P331S Fc domains employed in Example 3, as well as other Fc mutations and wild-type antibodies, were previously evaluated to assess binding to human Fcγ receptors, as follows.

SPR (Surface Plasmon Resonance) measurements were performed on a Biacore T100 apparatus (Biacore GE Healthcare) at 25° C. In all Biacore experiments HBS-EP+ (Biacore GE Healthcare) and 10 mM NaOH, 500 mM NaCl served as running buffer and regeneration buffer respectively. Sensorgrams were analyzed with Biacore T100 Evaluation software. Recombinant human FcR's (CD64, CD32a, CD32b, CD16a and CD16b) were cloned, produced and purified.

Antibodies tested included: antibodies having wild type human IgG1 domain, antibodies having a human IgG4 domain with S241P substitution, human IgG1 antibodies having a N297S substitution, human IgG1 antibodies having L234F/L235E/P331S substitutions, human IgG1 antibodies having L234A/L235E/P331S substitutions, human IgG1 antibodies having L234A/L235E/G237A/A330S/P331S substitutions, and human IgG1 antibodies having L234A/L235E/G237A/P331 S substitutions.

Antibodies were immobilized covalently to carboxyl groups in the dextran layer on a Sensor Chip CM5. The chip surface was activated with EDC/NHS (N-ethyl-N′-(3-dimethylaminopropyl) carbodiimidehydrochloride and N-hydroxysuccinimide (Biacore GE Healthcare)). Antibodies were diluted to 10 μg/ml in coupling buffer (10 mM acetate, pH 5.6) and injected until the appropriate immobilization level was reached (i.e. 800 to 900 RU). Deactivation of the remaining activated groups was performed using 100 mM ethanolamine pH 8 (Biacore GE Healthcare).

Monovalent affinity study was assessed following a classical kinetic wizard (as recommended by the manufacturer). Serial dilutions of soluble analytes (FcRs) ranging from 0.7 to 60 nM for CD64 and from 60 to 5000 nM for all the other FcRs were injected over the immobilized bispecific antibodies and allowed to dissociate for 10 min before regeneration. The entire sensorgram sets were fitted using the 1:1 kinetic binding model for CD64 and with the Steady State Affinity model for all the other FcRs.

The results are shown in Table 6 Results showed that while full length wild type human IgG1 bound to all human Fcγ receptors, and human IgG4 in particular bound significantly to FcγRI (CD64) (KD shown in Table 6), the L234A/L235E/G237A/A330S/P331 S substitutions and L234A/L235E/G237A/P331S substitutions abolished binding to CD64 as well as to CD16a.

### Example 5: Ability of ILT2 Blocking Antibodies to Enhance NK Cell Lysis

The ability of the anti-ILT2 antibodies to control ILT2-mediated inhibition of NK cell activation was determined by the capacity of ILT2-expressing KHYG cells described in Example 3 to lyse target cells in presence of antibodies. Effector cells were KHYG cells expressing ILT2 and GFP as control and target cells were ⁵¹Cr loaded K562 cell line expressing HLA-G. Effector and target cells were mixed at a ratio 1:10. Antibodies were pre-incubated 30 minutes at 37° C. with effector cells and then target cells were co-incubated 4 hours at 37° C. Specific lysis of target cells was calculated by the release of ⁵¹Cr in co-culture supernatant with a TopCount NXT (Perkin Elmer).

This experiment evaluated antibodies 3H5, 12D12, 26D8, 18E1, 27C10, 27H5, 1C11, 1D6, 9G1, 19F10a, 27G10 identified in Example 2, as well as commercially available antibodies GHI/75 (mouse IgG2b, Biolegend #333720), 292319 (mouse IgG2b, Bio-Techne #MAB20172), HP-F1 (mouse IgG1, eBioscience #16-5129-82), 586326 (mouse IgG2b, Bio-Techne #MAB30851) and 292305 (mouse IgG1, Bio-Techne #MAB20171).

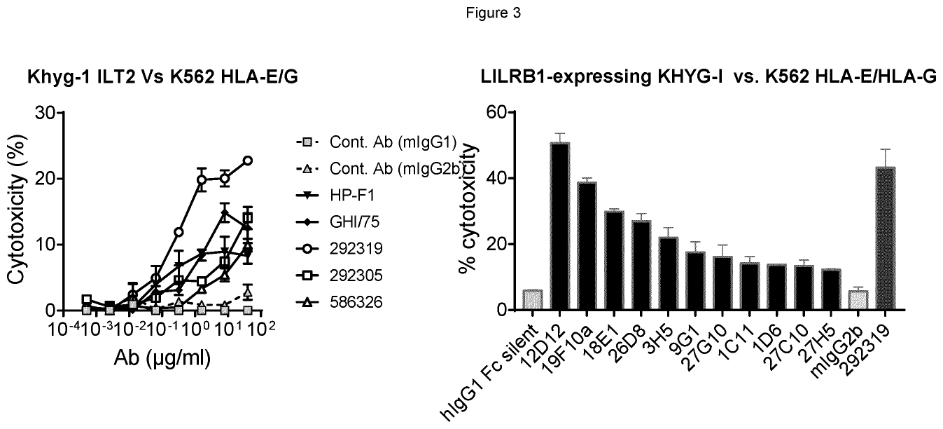

Results are shown in FIG. 3. Most of the ILT2/HLA-G blocking antibodies showed a significant increase in % cytotoxicity by the NK cell lines toward the K562-HLA-G tumor target cells. However, certain antibodies were particular potent at increasing NK cell cytotoxicity. Antibodies 12D12, 19F10a and commercial 292319 were significantly more effective than other antibodies in the ability to enhance NK cell cytotoxicity toward the target cells. Antibodies 18E1, 26D8, although less effective, displayed activity as enhancers of cytotoxicity, followed to a lesser extent by 3H5 and commercial antibody HP-F1. Other antibodies, including 27C10, 27H5, 1C11, 1D6, 9G1 and commercial antibodies 292305, 586326, GHI/75 were considerably less active than 18E1, 26D8 in their ability to induce cytotoxicity toward target cells.

### Example 6: Blockade of ILT2 Binding to HLA Class I Molecules

### HLA/ILT2 Blocking Assay

Ability of anti-ILT2 antibodies to block the interactions between HLA-G or HLA-A2 expressed at the surface of cell lines and recombinant ILT2 protein was assessed by flow cytometry. Briefly, BirA-tagged ILT2 protein was biotinylated to obtain 1 biotin molecule per ILT2 protein. APC-conjugated streptavidin (SA) was mixed with Biotinylated ILT2 protein (ratio 1 Streptavidin per 4 ILT2 protein) to form tetramers. Anti-ILT2 Abs (12D12, 18E1, 26D8) were incubated at 4° C. in staining buffer for 30 min with ILT2-SA tetramers. The Ab-ILT2-SA complexes were added on HLA-G or HLA-A2 expressing cells and incubated for 1 hour at 4° C. The binding of complexes on cells was evaluated on a Accury C6 flow cytometer equipped with an HTFC plate loader and analyzed using the FlowJo software.

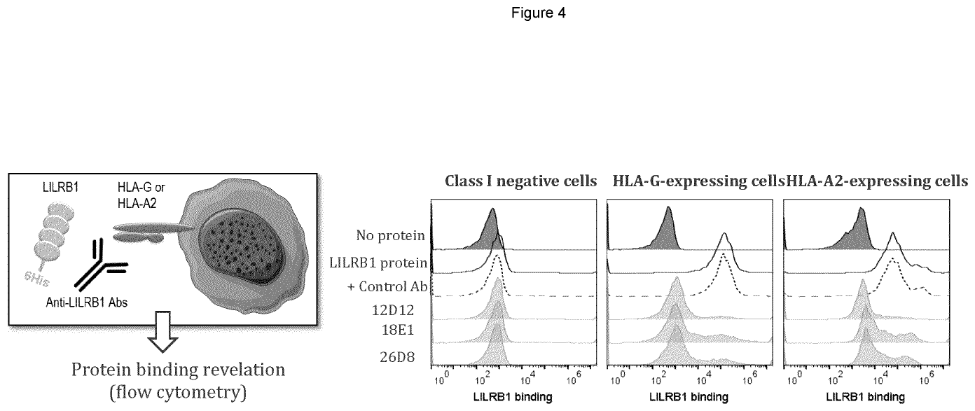

This assays permitted the identification of a panel of anti-ILT2 antibodies that were highly effective in blocking the interaction of ILT2 with its HLA class I ligand HLA-G. Antibodies 3H5, 12D12, 26D8, 18E1, 27C10, 27H5, 1C11, 1D6, 9G1, 19F10a and 27G10 all blocked ILT2 binding to HLA-G and HLA-A2. FIG. 4 shows representative results for antibodies 12D12, 18E1, and 26D8.

### Example 7: Antibody Titration on ILT2-Expressing Cells by Flow Cytometry

In order to explain the differences in NK cytotoxicity induction, unlabeled antibodies 3H5, 12D12, 26D8, 18E1, 27C10, 27H5, 1C11, 1D6, 9G1, 19F10a and 27G10 as well as the commercially available antibodies GHI/75, 292319, HP-F1, 586326 and 292305 were tested in experiments for binding to CHO cells modified to express human ILT-2. Cells were incubated with various concentrations of unlabeled anti-ILT2 antibodies from 30 μg/ml to 5×10⁻⁴ μg/ml, for 30 minutes at 4° C. After washes with staining buffer, cells were incubated for 30 min at 4° C. with Goat anti-human H+L AF488 secondary antibody (Jackson Immunoresearch #109-546-088) or Goat anti-mouse H+L AF488 secondary antibody for commercially available antibodies (Jackson Immuoresearch #115-545-146). Fluoresence was measured on an Accury C6 flow cytometer equipped with an HTFC plate loader.

Results are shown in Table 2, below. Except for antibody GHI/75 which had an EC50 in the range of 1-log higher that the other antibodies, the rest of the antibodies all showed comparable EC50 values, suggesting that differences binding affinity does not explain the observed differences in ability to enhance NK cell cytotoxicity.

TABLE 2

| | CHO-ILT2 | Primary NK |
|---|---|---|
| | cells | cells |
| Antibody | EC50 (μg/mL) | EC50 (μg/mL) |

| | | | | | |
|---|---|---|---|---|---|
| — | 3H5 | — | 0.35 | — | 0.48 |
| — | 12D12 | — | 0.36 | — | 0.09 |
| — | 26D8 | — | 0.15 | — | 0.11 |
| — | 18E1 | — | 0.12 | — | 0.11 |
| — | 27C10 | — | 0.25 | — | 0.33 |
| — | 27H5 | — | 0.52 | — | NA |
| — | 1C11 | — | 0.30 | — | 0.22 |
| — | 1D6 | — | 0.21 | — | 0.20 |
| — | 9G1 | — | 0.35 | — | 0.24 |
| — | 19F10a | — | 0.11 | — | 0.09 |
| — | 27G10 | — | 0.21 | — | 1.1 |
| — | HP-F1 | — | 0.56 | — | 0.09 |
| — | 292319 | — | 0.22 | — | 0.47 |
| — | 586326 | — | 0.13 | — | ND |
| — | GHI/75 | — | 5.39 | — | ND |
| — | 292305 | — | 0.27 | — | ND |

### Example 8: Monovalent Affinity Determination

Antibodies 3H5, 12D12, 26D8, 18E1, 27C10, 27H5, 1C11, 1D6, 9G1, 19F10a, and 27G10 as well as the commercially available antibodies GHI/75, 292319 and HP-F1 were tested for binding affinity to human ILT2 proteins.

SPR methods were used to test antibodies 3H5, 12D12, 26D8, 18E1, 27C10, 27H5, 1C11, 1D6, 9G1, 19F10a, 27G10 (all of human IgG1 isotype). Measurements were performed on a Biacore T200 apparatus (Biacore GE Healthcare) at 25° C. In all Biacore experiments HBS-EP+ (Biacore GE Healthcare) and NaOH 10 mM served as running buffer and regeneration buffer respectively. Sensorgrams were analyzed with Biacore T100 Evaluation software. Protein-A was purchased from (GE Healthcare). Human ILT2 recombinant proteins were cloned, produced and purified at Innate Pharma. Protein-A proteins were immobilized covalently to carboxyl groups in the dextran layer on a Sensor Chip CM5. The chip surface was activated with EDC/NHS (N-ethyl-N′-(3-dimethylaminopropyl) carbodiimidehydrochloride and N-hydroxysuccinimide (Biacore GE Healthcare)). Protein-A was diluted to 10 μg/ml in coupling buffer (10 mM acetate, pH 5.6) and injected until the appropriate immobilization level was reached (i.e. 2000 RU). Deactivation of the remaining activated groups was performed using 100 mM ethanolamine pH 8 (Biacore GE Healthcare). Anti-ILT2 antibodies at 1 μg/mL were captured onto the Protein-A chip and recombinant human ILT2 proteins were injected at 5 μg/mL over captured bispecific antibodies. For blank subtraction, cycles were performed again replacing ILT2 proteins with running buffer. The monovalent affinity analysis was conducted following a regular Capture-Kinetic protocol as recommended by the manufacturer (Biacore GE Healthcare kinetic wizard). Seven serial dilutions of human ILT2 proteins, ranging from 6.25 to 400 nM were sequentially injected over the captured antibodies and allowed to dissociate for 10 min before regeneration. The entire sensorgram sets were fitted using the 1:1 kinetic binding model.

OCTET analysis was used to evaluate antibodies GHI/75, 292319 and HP-F1, (all mouse isotypes). Measurements were performed on an Octet RED96 System (Fortebio). In all Biacore experiments Kinetics Buffer 10× (Fortebio) and Glycine 10 mM pH 1.8 served as running buffer and regeneration buffer respectively. Graphs were analyzed with Data Analysis 9.0 software. Anti-Mouse IgG Fc Capture (AMC) biosensors are used. Anti-ILT2 antibodies at 5 μg/mL were captured onto Anti-Mouse IgG Fc Capture (AMC) biosensors. Seven dilutions of recombinant human ILT2 proteins were injected (from 1000 nM to 15.625 nM for 292319 and HP-F1 and from 100 nM to 1.5625 nM for GHI-75). The curves were fitted using the model 1:1 Results are shown in Table 3, below. While antibody GHI/75 and 27H5 had a somewhat higher KD than other antibodies, the rest of the antibodies all showed comparable affinity and KD values, and the KD differences generally did not correlate to the differences in ability to enhance NK cell cytotoxicity. Binding affinity therefore did not explain the differences in the antibodies' ability to enhance NK cell cytotoxicity.

TABLE 3

| mAb | ka (1/Ms) | kd (1/s) | KD (M) |
|---|---|---|---|
| 3H5 | 4.4 | ka1: 2.8E+5 | kd1: 8.0E−3 |
| | | ka2: 8.7E−4 | kd2: 1.6E−4 |
| 12D12 | 1.0 | 4.3E+5 | 4.2E−4 |
| 26D8 | 0.4 | 6.2E+5 | 2.2E−4 |
| 18E1 | 0.2 | 7.5E+5 | 1.1E−4 |
| 27C10 | 0.2 | 1.4E+5 | 3.0E−4 |
| 27H5 | 13.9 | ka1: 6.6E+5 | kd1: 0.1 |
| | | ka2: 5.3E−3 | kd2: 4.2E−4 |
| 1C11 | 0.3 | 3.4E+5 | 1.1E−4 |
| 1D6 | 0.4 | 3.2E+5 | 1.2E−4 |
| 9G1 | 0.3 | 4.0E+5 | 1.3E−4 |
| 19F10a | 5.3 | 6.6E+5 | 3.5E−3 |
| 27G10 | 0.5 | 3.5E+5 | 1.8E−4 |
| GHI/75 | 28.1 | 1.3E+4 | 3.8E−4 |
| 292319 | 0.6 | 3.0E+5 | 1.7E−4 |
| HP-F1 | 2.3 | 4.6E+5 | 1.1E−3 |

### Example 9: Identification of Antibodies that Increase Cytotoxicity in Primary Human NK Cells

We considered the possibility that the inability of prior antibodies to neutralize ILT2 in NK cells might be related to differences in ILT2 expression in primary NK cells compared for example to highly selected or modified NK cell lines that express much higher levels of ILT2 at their surface. We studied and selected antibodies in primary NK cells from a number of healthy human donors. The effect of the anti-ILT2 antibodies of Example 5 was studied by activation assays by assessing CD137 surface expression on NK cells. In each case, primary NK cells (as fresh NK cells purified from donors) were used as effector cells and K562 cells (chronic myelogenous leukemia (CML)) expressing HLA-E/G were used as targets. The targets consequently thus expressed not only the ILT2 ligand HLA-G, but also HLA-E which is an HLA class I ligand expressed on the surface of a range of cancer cells and which can interact with inhibitory receptors on the surface of NK and CD8 T cells.

Briefly, the effect of the anti-ILT2 antibodies on NK cells activation was determined by analysis by flow cytometry of CD137 expression on total NK cells, ILT2-positive NK cells and ILT2-negative NK cells. Effector cells were primary NK cells (fresh NK cells purified from donors, incubation overnight at 37° C. before use) and target cells (K562 HLA-E/G cell line) were mixed at a ratio 1:1. The CD137 assay was carried out in 96 U well plates in completed RPMI, 200 μL final/well. Antibodies were pre-incubated 30 minutes at 37° C. with effector cells and then target cells were co-incubated overnight at 37° C. The following steps were: spin 3 min at 500 g; wash twice with Staining Buffer (SB); addition of 50 μL of staining Ab mix (anti-CD3 Pacific blue—BD Biosciences; anti-CD56-PE-Vio770—Miltenyi Biotec; anti-CD137-APC—Miltenyi Biotec; anti-ILT2-PE—clone HP-F1, eBioscience); incubation 30 min at 4° C.; wash twice with SB; resuspended pellet with SB; and fluorescence revealed with Canto II (HTS). Negative controls were NK cells vs K562-HLA-E/G alone and in presence of isotype control.

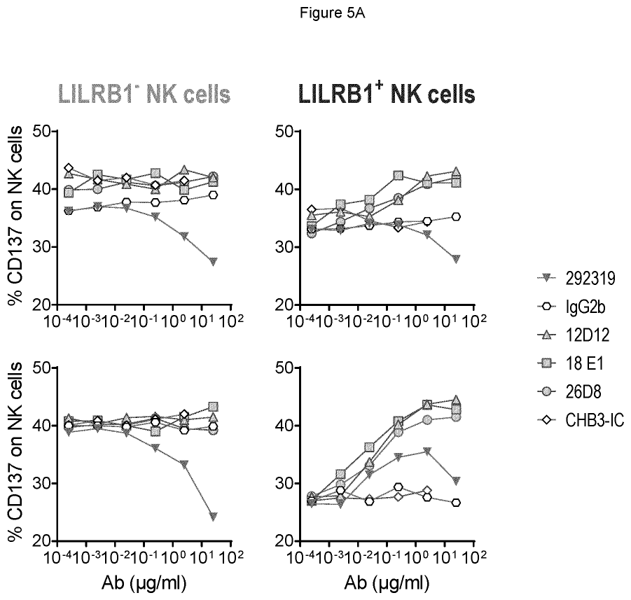

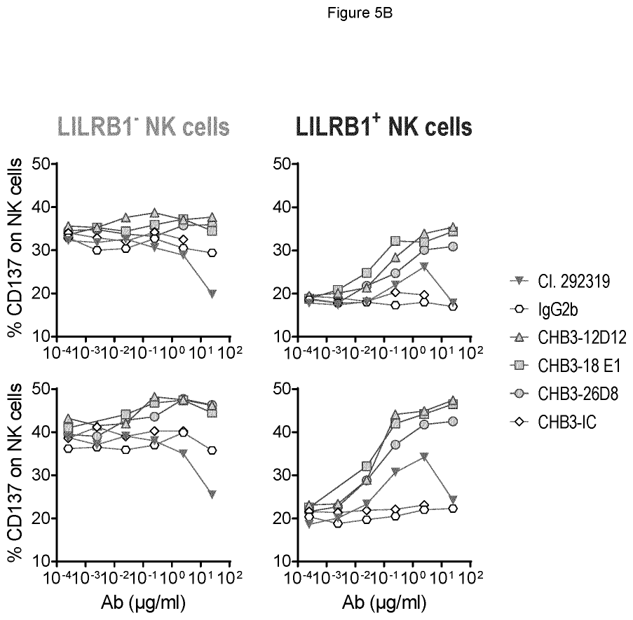

FIG. 5A is a representative figure showing the increase of % of total NK cells expressing CD137 mediated by anti-ILT2 antibodies using NK cells from two human donors and K562 tumor target cells made to express HLA-E and HLA-G. FIG. 5B is a representative figure showing the increase of % of ILT2-positive (left hand panel) and ILT2-negative (right hand panel) NK cells expressing CD137 mediated anti-ILT2 antibodies using NK cells from two human donors and an HLA-A2-expressing B cell line.

Surprisingly, it was observed that antibodies that were most effective in enhancing cytotoxicity of NK cell lines were not necessarily able to activate the primary human NK cells. Among the antibodies 12D12, 19F10a and 292319 that were most effective in enhancing cytotoxicity of NK cell lines, both 19F10a and 292319 substantially lacked the ability to activate the primary NK cells all, compared to isotype control antibodies.

On the other hand, antibodies 12D12, 18E1 and 26D8 showed strong activation of the primary NK cells. Study of ILT2-positive NK cells showed that these antibodies mediated a two-fold increase in activation of the NK cells toward the target cells. As a control, % of ILT2-negative NK cells expressing CD137 were not affected by the antibodies. Similarly, antibodies 2H2A, 3F5 and 48F12, which blocked ILT2 binding to HLA-G and HLA-A2, also showed strong activation of the primary NK cells.

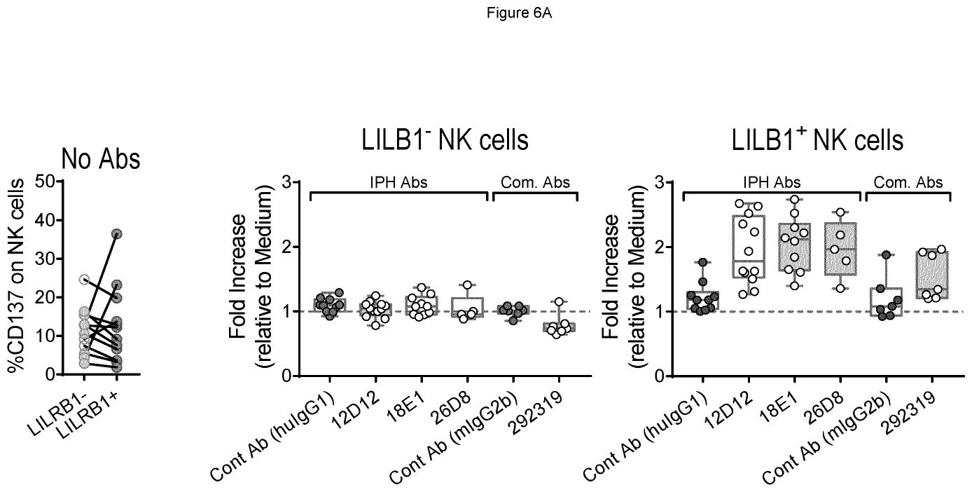

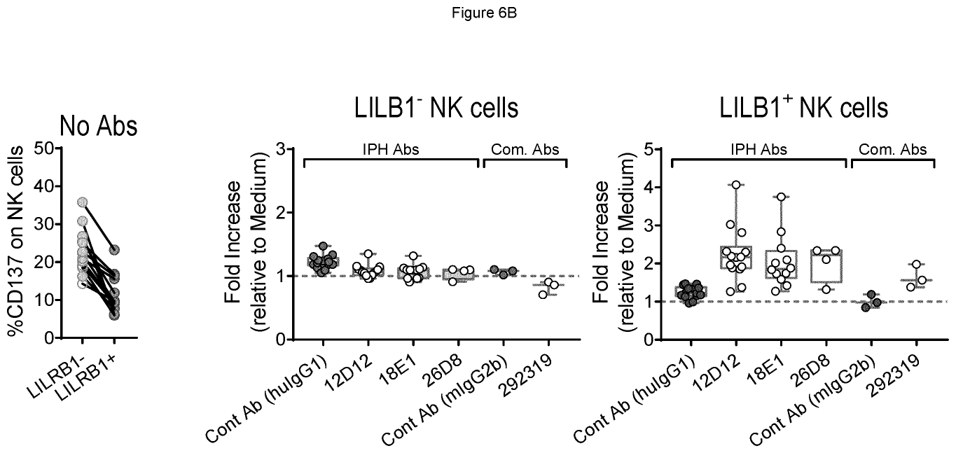

FIGS. 6A and 6B shows the ability of antibodies to enhance cytotoxicity of primary NK cells toward the tumor target cells in terms of fold-increase of cytotoxicity marker CD137. FIG. 6A shows the ability of antibodies to enhance NK cell activation in presence of HLA-G-expressing target cells using primary NK cells from 5-12 different donors against HLA-G and HLA-E expressing K562 target cells. FIG. 6A shows the ability of antibodies to enhance NK cell activation in presence of HLA-G-expressing target cells using primary NK cells from 3-14 different donors against the HLA-A2 expressing target B cells. In each case 12D12, 18E1 and 26D8 had greater enhancement of NK cytotoxicity compared to one of the antibodies (292319) which was among the antibodies showing strongest enhancement of NK cytotoxicity when using NK cell lines in Example 5.

### Example 10: Epitope Mapping

### Anchored ILT2 Domain Fragment Proteins

### Generation of ILT2 Proteins

Nucleic acid sequences encoding different human ILT2 domains D1 (corresponding to residues 24-121 of the sequence shown in SEQ ID NO: 1), D2 (corresponding to residues 122-222 of the sequence shown in SEQ ID NO: 1), D3 (corresponding to residues 223-321 of the sequence shown in SEQ ID NO: 1), D4 (corresponding to residues 322-458 of the sequence shown in SEQ ID NO: 1), and combinations thereof, were amplified by PCR using the primers described in the Table below. The PCR products were inserted into an expression vector at appropriate restriction sites. A heavy chain peptide leader was used and a V5 tag was added at the N-terminal and expression at the surface of cells was confirmed by flow cytometry. For all of the domains that were not followed by a 04 domain, a 0024 GPI anchor was added to permit anchoring at the cell membrane. The amino acid sequences of the resulting different human ILT2 domain fragment-containing proteins are shown below in Table 4, below. The vectors were then transfected into the CHO cell line to obtain stable clones expressing the different ILT2 domain proteins at the cell surface.

TABLE 4

| | | SEQ ID |
|---|---|---|
| Description | Amino acid sequence | NO |
| D1 domain | TGVHSGKPIPNPLLGLDSTGHLPKPTLWAEPGSVITQGSPVTLRCQGGQETQ | 112 |
| | EYRLYREKKTALWITRIPQELVKKGQFPIPSITWEHAGRYRCYYGSDTAGRS | |
| | ESSDPLELVVTGAGALQSTASLFVVSLSLLHLYS | |
| D2 domain | TGVHSGKPIPNPLLGLDSTYIKPTLSAQPSPVVNSGGNVILQCDSQVAFDGF | 113 |
| | SLCKEGEDEHPQCLNSQPHARGSSRAIFSVGPVSPSRRWWYRCYAYDSNSPY | |
| | EWSLPSDLLELLVLGVGALQSTASLFVVSLSLLHLYS | |
| D3 domain | TGVHSGKPIPNPLLGLDSTSKKPSLSVQPGPIVAPEETLTLQCGSDAGYNRF | 114 |
| | VLYKDGERDFLQLAGAQPQAGLSQANFTLGPVSRSYGGQYRCYGAHNLSSEW | |
| | SAPSDPLDILIAGQGALQSTASLFVVSLSLLHLYS | |
| D4 domain | TGVHSGKPIPNPLLGLDSTFYDRVSLSVQPGPTVASGENVTLLCQSQGWMQT | 115 |
| | FLLTKEGAADDPWRLRSTYQSQKYQAEFPMGPVTSAHAGTYRCYGSQSSKPY | |
| | LLTHPSDPLELVVSGPSGGPSSPTTGPTSTSGPEDQPLTPTGSDPQSGLGRH | |
| | LGVVIGILVAVILLLLLLLLLFLILRHRRQGKHWTSTQRKADFQHPAGAVGP | |
| | EPTDRGLQWRSSPAADAQEENLYAAVKHTQPEDGVEMDTRSPHDEDPQAVTY | |
| | AEVKHSRPRREMASPPSPLSGEFLDTKDRQAEEDRQMDTEAAASEAPQDVTY | |
| | AQLHSLTLRREATEPPPSQEGPSPAVPSIYATLAIH | |
| D1-D2 | TGVHSGKPIPNPLLGLDSTGHLPKPTLWAEPGSVITQGSPVTLRCQGGQETQ | 116 |
| domain | EYRLYREKKTALWITRIPQELVKKGQFPIPSITWEHAGRYRCYYGSDTAGRS | |
| | ESSDPLELVVTGAYIKPTLSAQPSPVVNSGGNVILQCDSQVAFDGFSLCKEG | |
| | EDEHPQCLNSQPHARGSSRAIFSVGPVSPSRRWWYRCYAYDSNSPYEWSLPS | |
| | DLLELLVLGVGALQSTASLFVVSLSLLHLYS | |
| D2-D3 | TGVHSGKPIPNPLLGLDSTYIKPTLSAQPSPVVNSGGNVILQCDSQVAFDGF | 117 |
| domain | SLCKEGEDEHPQCLNSQPHARGSSRAIFSVGPVSPSRRWWYRCYAYDSNSPY | |
| | EWSLPSDLLELLVLGVSKKPSLSVQPGPIVAPEETLTLQCGSDAGYNRFVLY | |
| | KDGERDFLQLAGAQPQAGLSQANFTLGPVSRSYGGQYRCYGAHNLSSEWSAP | |
| | SDPLDILIAGQGALQSTASLFVVSLSLLHLYS | |
| D3-D4 | TGVHSGKPIPNPLLGLDSTSKKPSLSVQPGPIVAPEETLTLQCGSDAGYNRF | 118 |
| domain | VLYKDGERDFLQLAGAQPQAGLSQANFTLGPVSRSYGGQYRCYGAHNLSSEW | |
| | SAPSDPLDILIAGQFYDRVSLSVQPGPTVASGENVTLLCQSQGWMQTFLLTK | |
| | EGAADDPWRLRSTYQSQKYQAEFPMGPVTSAHAGTYRCYGSQSSKPYLLTHP | |
| | SDPLELVVSGPSGGPSSPTTGPTSTSGPEDQPLTPTGSDPQSGLGRHLGVVI | |
| | GILVAVILLLLLLLLLFLILRHRRQGKHWTSTQRKADFQHPAGAVGPEPTDR | |
| | GLQWRSSPAADAQEENLYAAVKHTQPEDGVEMDTRSPHDEDPQAVTYAEVKH | |
| | SRPRREMASPPSPLSGEFLDTKDRQAEEDRQMDTEAAASEAPQDVTYAQLHS | |
| | LTLRREATEPPPSQEGPSPAVPSIYATLAIH | |
| D1-D2-D3 | TGVHSGKPIPNPLLGLDSTGHLPKPTLWAEPGSVITQGSPVTLRCQGGQETQ | 119 |
| domain | EYRLYREKKTALWITRIPQELVKKGQFPIPSITWEHAGRYRCYYGSDTAGRS | |
| | ESSDPLELVVTGAYIKPTLSAQPSPVVNSGGNVILQCDSQVAFDGFSLCKEG | |
| | EDEHPQCLNSQPHARGSSRAIFSVGPVSPSRRWWYRCYAYDSNSPYEWSLPS | |
| | DLLELLVLGVSKKPSLSVQPGPIVAPEETLTLQCGSDAGYNRFVLYKDGERD | |
| | FLQLAGAQPQAGLSQANFTLGPVSRSYGGQYRCYGAHNLSSEWSAPSDPLDI | |
| | LIAGQGALQSTASLFVVSLSLLHLYS | |
| D2-D3-D4 | TGVHSGKPIPNPLLGLDSTYIKPTLSAQPSPVVNSGGNVILQCDSQVAFDGF | 120 |
| domain | SLCKEGEDEHPQCLNSQPHARGSSRAIFSVGPVSPSRRWWYRCYAYDSNSPY | |
| | EWSLPSDLLELLVLGVSKKPSLSVQPGPIVAPEETLTLQCGSDAGYNRFVLY | |
| | KDGERDFLQLAGAQPQAGLSQANFTLGPVSRSYGGQYRCYGAHNLSSEWSAP | |
| | SDPLDILIAGQFYDRVSLSVQPGPTVASGENVTLLCQSQGWMQTFLLTKEGA | |
| | ADDPWRLRSTYQSQKYQAEFPMGPVTSAHAGTYRCYGSQSSKPYLLTHPSDP | |
| | LELVVSGPSGGPSSPTTGPTSTSGPEDQPLTPTGSDPQSGLGRHLGVVIGIL | |
| | VAVILLLLLLLLLFLILRHRRQGKHWTSTQRKADFQHPAGAVGPEPTDRGLQ | |
| | WRSSPAADAQEENLYAAVKHTQPEDGVEMDTRSPHDEDPQAVTYAEVKHSRP | |
| | RREMASPPSPLSGEFLDTKDRQAEEDRQMDTEAAASEAPQDVTYAQLHSLTL | |
| | RREATEPPPSQEGPSPAVPSIYATLAIH | |

### Results

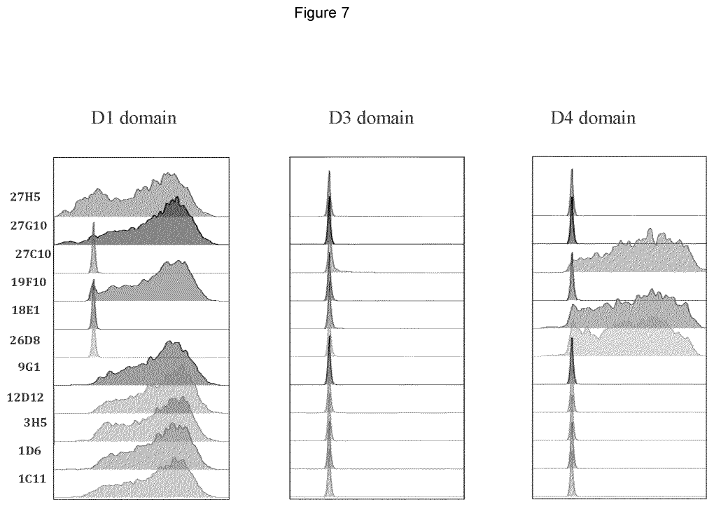

The ILT2 selective antibodies were tested for their binding to the different anchored ILT2 fragments by flow cytometry. 3H-5, 12012 and 27H-5 all bound to the D1 domain of ILT2. These antibodies bound to all cells that expressed proteins that contained the D1 domain of ILT2, (the proteins of SEQ ID NOS: 112, 116 and 119) without binding to any of the cells that expressed the ILT2 proteins that lacked the D1 domain (the proteins of SEQ ID NOS: 113-115, 117, 118 and 120). The antibodies 3H-5, 12012 and 27H-5 thus bind to a domain of ILT2 defined by residues 24-121 of the sequence shown in SEQ ID NO: 1 (also referred to as domain D1). Antibodies 2608, 18E1 and 27010 all bound to the D4 domain of ILT2. These antibodies bound to all cells that expressed proteins that contained the D4 domain of ILT2, (the proteins of SEQ ID NOS: 115, 118 and 120) without binding to any of the cells that expressed the ILT2 proteins that lacked the D4 domain (the proteins of SEQ ID NOS: 112-114, 116, 117, or 119). The antibodies 2608, 18E1 and 27010 thus bind to a domain of ILT2 defined by residues 322-458 of the sequence shown in SEQ ID NO: 1. FIG. 7 shows a representative example binding of the antibodies to the anchored ILT2 domain D1 fragment protein of SEQ ID NO: 112 (left hand panel), the D3 domain fragment protein of SEQ ID NO: 114 (middle panel), and the D4 domain protein of SEQ ID NO: 115 (right hand panel).

### ILT2 Point Mutation Study

The identification of antibodies that bound ILT2 without binding to the closely related ILT6 permitted the design of ILT2 mutations on amino acids exposed and different between ILT2 and ILT6. Anti-ILT2 antibodies that did not cross-react on ILT6 could then be mapped for loss of binding to different ILT2 mutants having amino acid substitutions in the D1, D2 or D4 domains of ILT2.

### Generation of ILT2 Mutants

ILT2 mutants were generated by PCR. The sequences amplified were run on agarose gel and purified using the Macherey Nagel PCR Clean-Up Gel Extraction kit (reference 740609). The purified PCR products generated for each mutant were then ligated into an expression vector, with the ClonTech InFusion system. The vectors containing the mutated sequences were prepared as Miniprep and sequenced. After sequencing, the vectors containing the mutated sequences were prepared as Midiprep using the Promega PureYield™ Plasmid Midiprep System. HEK293T cells were grown in DMEM medium (Invitrogen), transfected with vectors using Invitrogen's Lipofectamine 2000 and incubated at 37° C. in a CO2 incubator for 48 hours prior to testing for transgene expression. Mutants were transfected in Hek-293T cells, as shown in the table below. The targeted amino acid mutations are shown in the Table 5 below, listing the residue present in wild-type ILT2/position of residue/residue present in mutant ILT2, with position reference being to either the ILT2 protein lacking leader peptide shown in SEQ ID NO: 2 in the left column, or to the ILT2 protein with leader peptide shown in SEQ ID NO: 1 in the right column.

TABLE 5

| | Amino acid substitutions with | Amino acid substitutions with |
|---|---|---|
| | reference to ILT2 lacking leader | reference to ILT2 having leader |
| Mutant | peptide of SEQ ID NO: 2 | peptide of SEQ ID NO: 1 |
| 1 | G29S - Q30L - T32A - Q33A - D80H | G52S - Q53L - T55A - Q56A - D103H |
| 2 | E34A - R36A - Y76I - A82S - R84L | E57A - R59A - Y99I- A105S - R107L |
| 3 | Y99A - I100S - V126S - A127S - | Y122A - I123S - V149S - A150S - |
| | D129A - N180R - S181A - E184G | D152A - N203R - S204A - E207G |
| 3b | Q18A - W67A - Y99A - I100S - V126S - | Q41A - W90A - Y122A - I123S - V149S - |
| | S181A - E184G | S204A - E207G |
| 4 | S132A - L145S - N146A - Q148H - | S155A - L168S - N169A - Q171H - |
| | P149S | P172S |
| 5 | A127S - D129A - Q148H - R152A - | A150S - D152A - Q171H - R175A - |
| | N180R | N203R |
| 6 | Q107L - P108A - I119A - R156A | Q130L - P131A - I142A - R179A |
| 7 | P166A - R169A - W171S - L191A - | P189A - R192A - W194S - L214A - |
| | E193G - L195S - L197P | E216G - L218S - L220P |
| 8 | V111S - N113A - L195S - L197P | V134S - N136A - L218S - L220P |
| 4-1 | F299I - Y300R - D301A - W328G - | F322I - Y323R - D324A - W351G - |
| | Q378A - K381N | Q401A - K404N |
| 4-1b | Y300R - D301A - R302A - S304F - | Y323R - D324A - R325A - S327F - |
| | H387A - D390A | H410A - D413A |
| 4-2 | W328G - Q330H - R347A - T349A - | W351G - Q353H - R370A - T372A - |
| | Y350S - Y355A | Y373S - Y378A |
| 4-3 | Q324A - Q326S - S352A - Q353H - | Q347A - Q349S - S375A - Q376H - |
| | K354A | K377A |
| 4-4 | Q308A - P309G - N318A - T320A - | Q331A - P332G - N341A - T343A - |
| | E358S - G362S | E381S - G385S |
| 4-5 | D341A - D342S - W344L - R345A - | D364A - D365S - W367L - R368A - |
| | R347A | R370A |

### Results

The ILT2 selective antibodies were tested for their binding to each of mutants by flow cytometry. A first experiment was performed to determine antibodies that lose their binding to one or several mutants at one concentration. To confirm a loss of binding, titration of antibodies was done on antibodies for which binding seemed to be affected by the ILT2 mutations. A loss or decrease of binding for a test antibody indicated that one or more, or all of, the residues of the particular mutant are important to the core epitope of the antibodies, and thereby permitted the region of binding of ILT2 to be identified.

Antibodies 3H-5, 12012 and 27H-5 bound an epitope in domain D1 of ILT2, as these three antibodies lost binding to mutant 2 having amino acid substitutions at residues 34, 36, 76, 82 and 84 (substitutions E34A, R36A, Y76I, A82S, R84L) in the domain 1 (D1 domain) of ILT2. 12D12 and 27H5 did not lose binding to any other mutant, however 3H5 also had a decrease (partial loss) of binding to mutant 1 having amino acid substitutions at residues 29, 30, 33, 32, 80 (substitutions G29S, Q30L, Q33A, T32A, D80H).

Antibodies 2H2A, 48F12 and 3F5 bound an epitope in domain D1 of ILT2, with loss of binding to mutant 2 having amino acid substitutions at residues 34, 36, 76, 82 and 84 (substitutions E34A, R36A, Y76I, A82S, R84L) in the domain 1 (D1 domain) of ILT2.

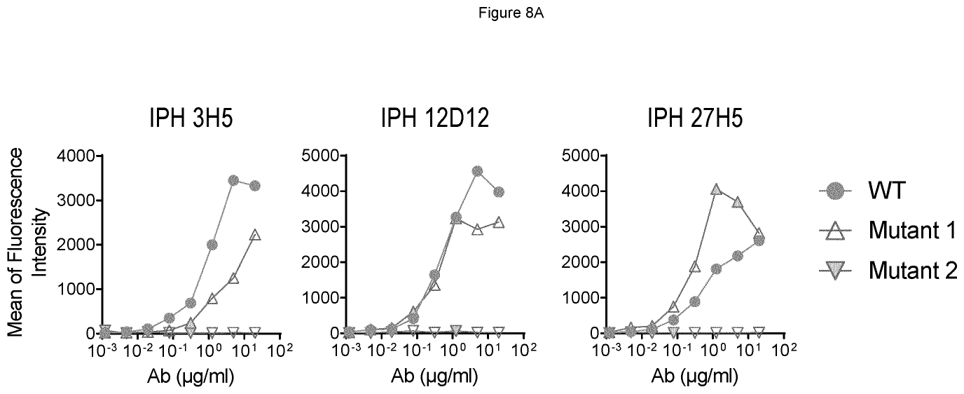

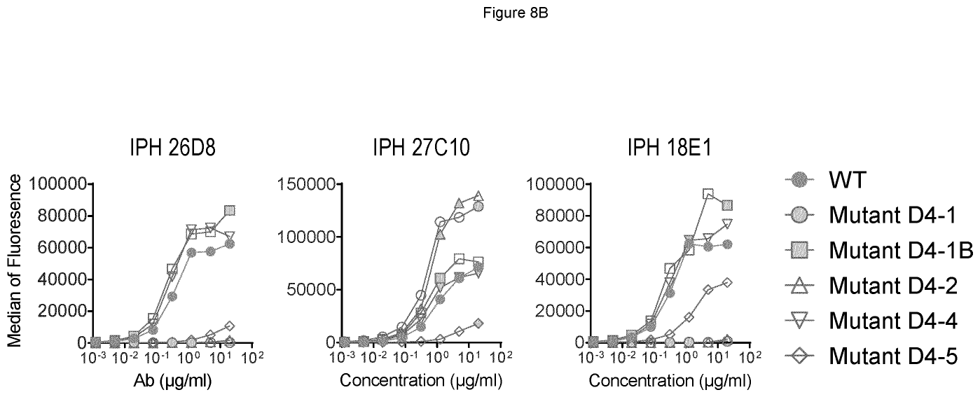

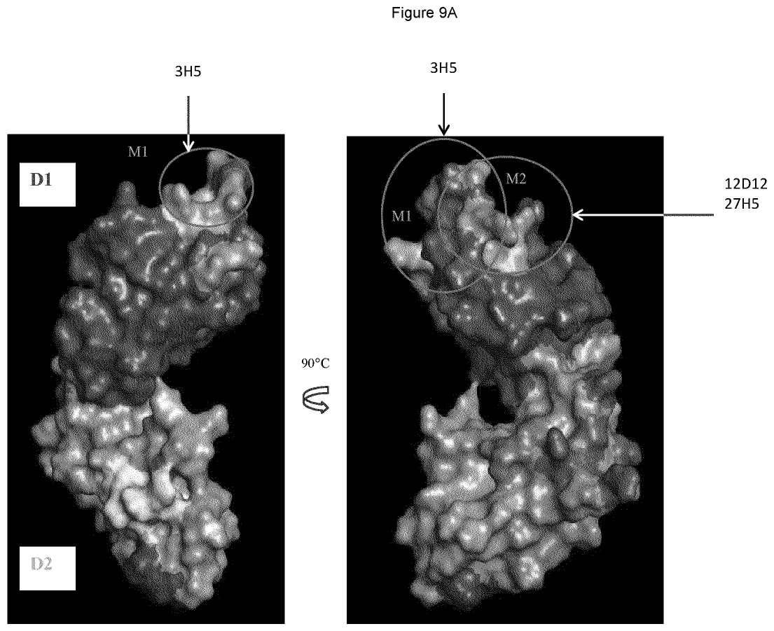

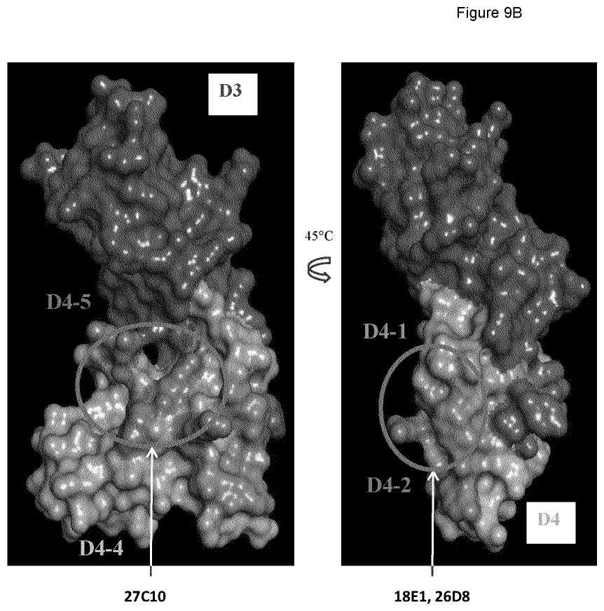

FIG. 8A shows a representative example of titration of antibodies 3H5, 12D12 and 27H5 for binding to mutants 1 and 2 by flow cytometry. FIG. 9A shows a model representing a portion of the ILT2 molecule that includes domain 1 (top portion, shaded in dark gray) and domain 2 (bottom, shaded in light gray). The figure shows the binding site of the antibodies as defined by the amino acid residues substituted in mutant 1 (M1) and mutant 2 (M2).

Antibodies 26D8, 18E1 and 27C10 all bound an epitope in domain D4 of ILT2. Antibodies 26D8 and 18E1 lost binding to mutants 4-1 and 4-2. Mutant 4-1 has amino acid substitutions at residues 299, 300, 301, 328, 378 and 381 (substitutions F299I, Y300R, D301A, W328G, Q378A, K381N). Mutant 4-2 has amino acid substitutions at residues 328, 330, 347, 349, 350 and 355 (substitutions W328G, Q330H, R347A, T349A, Y350S, Y355A). 26D8 furthermore lost binding to mutant 4-5, while antibody 18E1 had a decrease in binding (but not complete loss of binding) to mutant 4-5. 27C10 also lost binding to mutant 4-5, but not to any other mutant. Mutant 4-5 has amino acid substitutions at residues 341, 342, 344, 345 and 347 (substitutions D341A, D342S, W344L, R345A, R347A). 26D8 and 18E1 did not lose binding to any other mutants.

FIG. 8B shows a representative example of titration of antibodies 26D8, 18E1 and 27C10 for binding to D4 domain mutants 4-1, 4-1b, 4-2, 4-4 and 4-5 by flow cytometry FIG. 9B shows a model representing a portion of the ILT2 molecule that includes domain 3 (top portion, shaded in dark gray) and domain 4 (bottom, shaded in light gray). The figure shows the binding site of the antibodies as defined by the amino acid residues substituted in mutants, 4-1, 4-2 and 4-5 which are all located within domain 4 of ILT2. Antibodies 26D8, 18E1 which potentiate the cytotoxicity of primary NK cells bind the site defined by mutants 4-1 and 4-2 without binding to the site defined by mutant 4-5, while antibodies 27C10 which did not potentiate the cytotoxicity of primary NK cells binds to the site defined by mutant 4-5.

### Example 11: ILT2 in Urothelial Cancer

Potentiation of Cytotoxicity in Primary NK Cells from Urothelial Cancers Patients Towards HLA-A2-Expressing Cells

The effect of the anti-ILT2 antibodies on NK cell activation was determined by analysis by flow cytometry of CD137 expression on total NK cells, ILT2-positive NK cells and ILT2-negative NK cells from human urothelial carcinoma patients.

Effector cells were primary NK cells (fresh NK cells purified from human urothelial cancer donors, incubation overnight at 37° C. before use) and target cells (HLA-A2-expressing B cell line reference B104) were mixed at a ratio 1:1. The CD137 assay was carried out in 96 U well plates in completed RPMI, 200 μL final/well. Antibodies were pre-incubated 30 minutes at 37° C. with effector cells and then target cells were co-incubated overnight at 37° C. The following steps were: spin 3 min at 500 g; wash twice with Staining Buffer (SB); addition of 50 μL of staining Ab mix (anti-CD3 Pacific blue—BD Biosciences; anti-CD56-PE-Vio770—Miltenyi Biotec; anti-CD137-APC—Miltenyi Biotec; anti-ILT2-PE—clone HP-F1, eBioscience); incubation 30 min at 4° C.; wash twice with SB; resuspended pellet with SB; and fluorescence revealed with Canto II (HTS). Negative controls were NK cells vs target cells alone and in presence of isotype control.

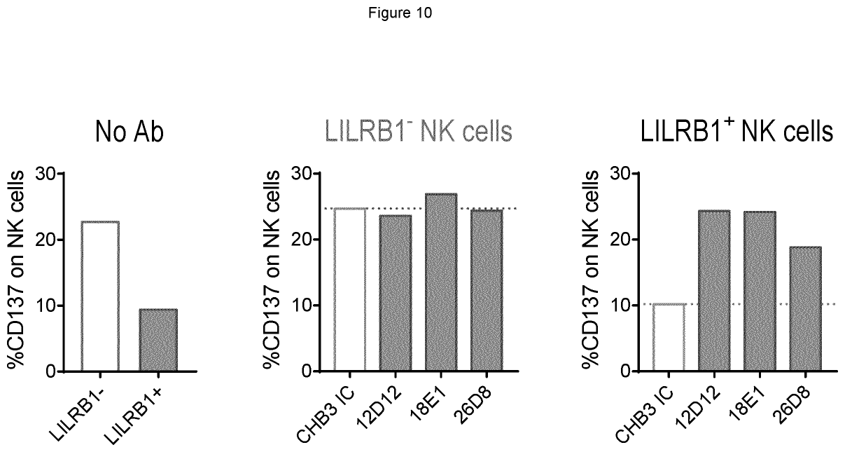

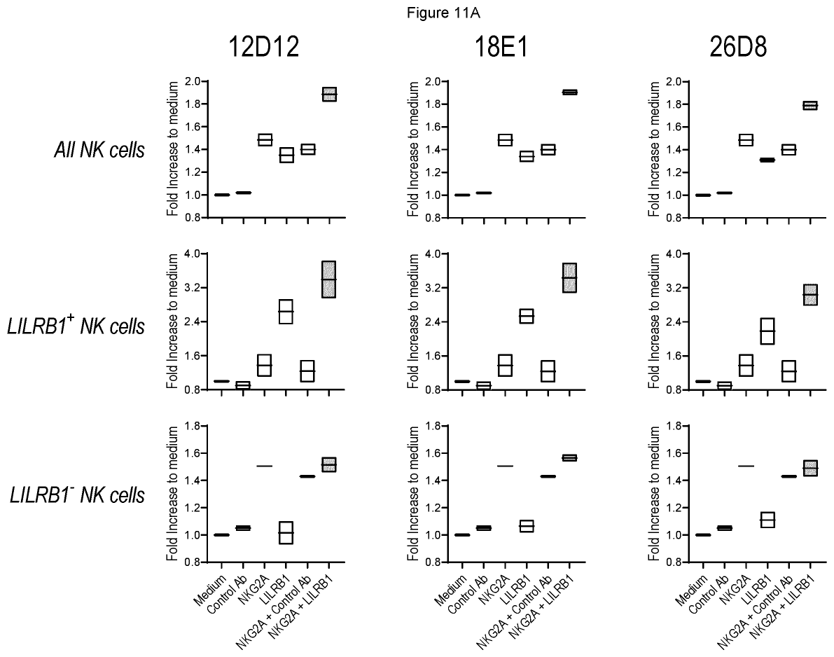

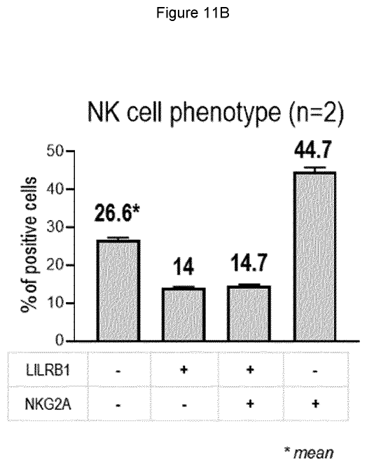

FIG. 10 shows the % of ILT2-positive (right hand panel) and ILT2-negative (middle panel) NK cells from urothelial cancer patients expressing CD137 following incubation with anti-ILT2 antibodies 12D12, 18E1 and 26D8 and the HLA-A2-expressing B cells. Each of the anti-ILT2 antibodies 12D12, 18E1 and 26D8 caused a more than 2-fold increase in NK cell cytotoxicity.

### Example 12: Anti-ILT2 Combined with Antibodies that Block the NKG2A/HLA-E Interaction

### Anti-NKG2A Antibodies and Anti-ILT2 Antibodies Together Strongly Enhance NK Cell Cytotoxicity Towards Tumor Cells

The effect of the anti-ILT2 antibodies on NK cell activation was determined by analysis by flow cytometry of CD137 expression on NK cells, ILT2-positive NK cells and ILT2-negative NK cells from human tumor cells.

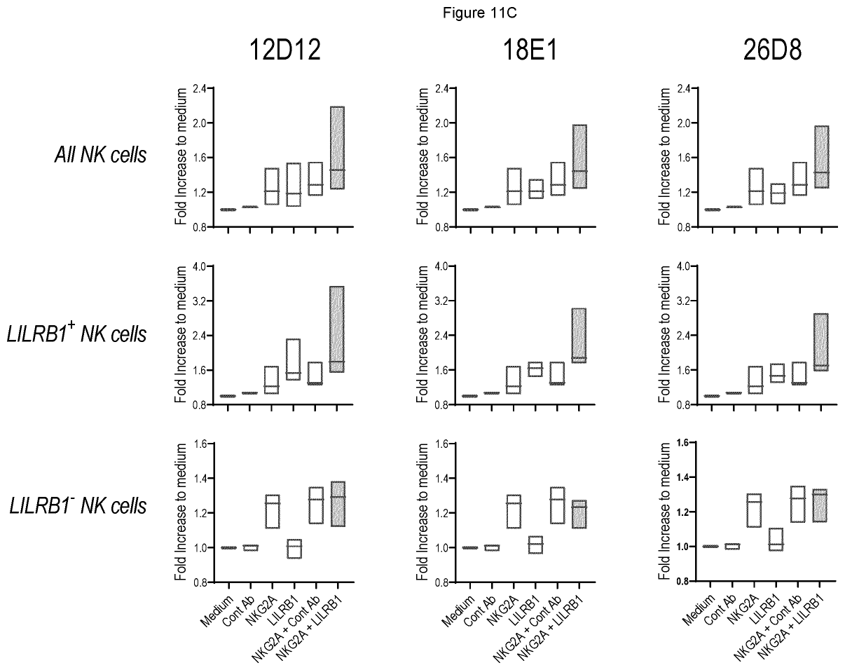

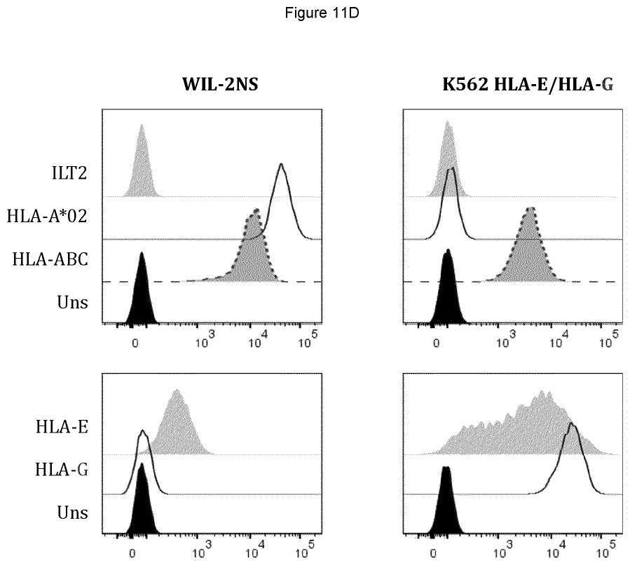

Tumor target cells included K562 cells transfected with HLA-E and HLA-G1, as well as WIL2-NS tumor target cells not transfected with HLA-E or HLA-G, in which ILT-2 was silenced. Phenotyping of WIL-2NS and K562 tumor target cells for expression of ILT2 ligands is shown in FIG. 11D. Effector cells (fresh NK cells purified from human healthy donors) and tumor target cells were mixed at a ratio 1:1. The CD137 assay was carried out in 96 U well plates in completed RPMI, 200 μL final/well. Antibodies used included anti-ILT-2 antibodies 12D12, 18E1 and 26D8, anti-NKG2A neutralizing antibody IPH2201 having the heavy and light chain amino acid sequences of SEQ ID NOS: 65 and 69, and isotype control antibody. Antibodies were pre-incubated 30 minutes at 37° C. with effector cells and then target cells were co-incubated overnight at 37° C. The following steps were: spin 3 min at 400 g; wash twice with Staining Buffer (SB); addition of 50 μL of staining Ab mix (anti-CD3 Pacific blue—BD Biosciences; anti-CD56-PE-Vio770—Miltenyi Biotec; anti-CD137-APC—Miltenyi Biotec; anti-ILT2-PE—clone HP-F1, eBioscience); incubation 30 min at 4° C.; wash twice with SB; resuspended pellet with Cellfix—Becton Dickinson; and fluorescence revealed with a FACS Canto II flow cytometer (Becton Dickinson). Negative controls were NK cells vs target cells alone and in presence of isotype control.

The study aimed to compare the effect of the anti-ILT2 antibodies to anti-NKG2A antibodies, and different antibodies were tested together with negative controls, and well as different samples in combination. The anti-ILT2 antibodies were able to mediate a strong increase of NK cell cytotoxicity which was comparable to that observed with blocking anti-NKG2A antibodies. Surprisingly, the combination of anti-ILT2 antibodies and anti-NKG2A antibodies resulted in much stronger activation of total NK cell activation that either agent was able to mediate on its own. FIG. 11A shows the fold increase (compared to medium) in activation of NK cells following incubation with anti-ILT2 antibodies, anti-NKG2A antibodies, or the combination of anti-ILT2 and anti-NKG2A antibodies, and the HLA-E/HLA-G-expressing K562 tumor target cells, in two human donors. The combination of anti-ILT2 and anti-NKG2A resulted in NK cytotoxicity that was significantly higher that of the NK cell cytotoxicity of each of the anti-ILT2 or anti-NKG2A agents alone. The combination increased NK cell cytotoxicity compared to either agent alone not only in the entire NK cell population, but also in the LILRB1+ population of NK cells, suggesting that NKG2A has a significant role in restricting the cytotoxicity of these cells. NK cell phenotyping of the two human donors is shown in FIG. 11B, showing that LILRB1 and NKG2A expression is found in about 15% of NK cells but is also in large part non-overlapping and that these receptors together identify about three quarters of total NK cells.

FIG. 11C shows the fold increase (compared to medium) in activation of NK cells following incubation with anti-ILT2 antibodies, anti-NKG2A antibodies, or the combination of anti-ILT2 and anti-NKG2A antibodies, and the WIL-2NS tumor target cells, in four human donors. Again, the combination of anti-ILT2 and anti-NKG2A resulted in NK cytotoxicity that was significantly higher that of the NK cell cytotoxicity of each of the anti-ILT2 or anti-NKG2A agents alone.

### Example 13: ILT2 in Clear Cell Renal Carcinoma

Correlation of ILT2 Expression with Survival in Human CCRCC Patients

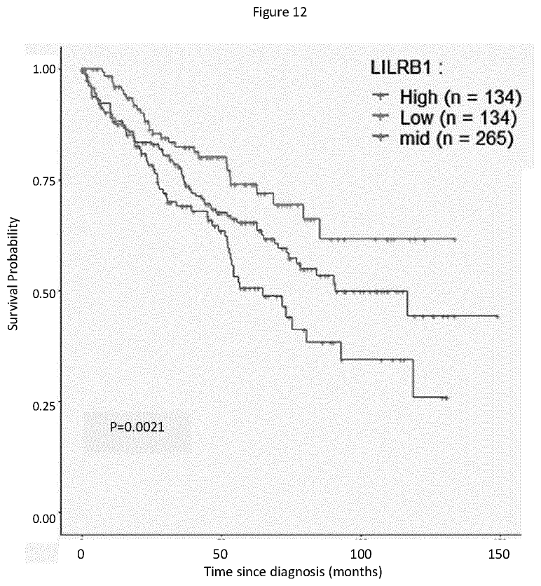

A study of ILT2 gene expression study was carried out using Cancer Genome Atlas (a collaboration between the National Cancer Institute and National Human Genome Research Institute) based on multi-dimensional maps of the key genomic changes in different types of cancer. Levels of expression (indicated as high or low) were considered, taking account of disease stage and time. For ILT2 and kidney clear cell renal cell carcinoma (CCRCC) patients were divided in 3 groups (high, mid and low ILT2 gene expression) according to the p-value of the Cox regression (each group must contain at least 10% of patients). Survival probability curves were drawn for each of the 3 groups. Statistical survival differences between low, mid and high ILT2 expression were observed for CCRCC samples, with high-expressing ILT2 exhibiting lower survival. FIG. 12 shows low ILT2 expressing samples (top line), medium ILT2-expressing samples (middle line) and high ILT2-expressing samples (bottom line). The results show that increased ILT2 expression correlates with lower survival probability. The high ILT2-expressing samples were associated with lower survival probability compared to medium and low ILT2 expressing samples.

TABLE 6

| | | | | L234A/ | | | |
|---|---|---|---|---|---|---|---|
| | | | | L235E/ | L234A/ | Wild type | |
| | | L234F/ | L234A/ | G237A/ | L235E/ | human | Human IgG4 |
| | | L235E/ | L235E/ | A330S/ | G237A/ | IgG1 | antibody |
| Human Fc | N297S | P331S | P331S | P331S | P331S | antibody | with S241P |
| receptor | KD (nM) | KD (nM) | KD (nM) | KD (nM) | KD (nM) | KD (nM) | KD (nM) |
| CD64 | 278 | 933 | 1553 | No binding | No binding | 12.74 | 96.83 |
| CD32a | No binding | 14250 | 19900 | 18190 | 16790 | 2075 | 3218 |
| CD32b | No binding | 17410 | 79830 | 21800 | 16570 | 3914 | 2659 |
| CD16a(F) | No binding | 35580 | No binding | No binding | No binding | 961.9 | Low |
| | | | | | | | binding |
| CD16a(V) | No binding | 8627 | 9924 | No binding | No binding | 733.7 | 8511 |
| CD16b | No binding | No binding | No binding | No binding | No binding | 15020 | Low |
| | | | | | | | binding |
| FcRn | 712 | 627 | 1511 | 714 | 758 | 1272 | 1176 |

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate). Where “about” is used in connection with a number, this can be specified as including values corresponding to +/−10% of the specified number.

The description herein of any aspect or embodiment of the invention using terms such as “comprising”, “having,” “including,” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

1-20. (canceled)

21. A pharmaceutical composition comprising an antibody that binds a human ILT-2 polypeptide and neutralizes the inhibitory activity of ILT-2, an antibody that neutralizes the inhibitory activity of a human NKG2A polypeptide, and a pharmaceutically acceptable carrier.

22. The pharmaceutical composition of claim 21, wherein the antibody that binds ILT-2 is capable of inhibiting the interaction between an ILT-2 polypeptide and a HLA-G and/or HLA-A2 polypeptide expressed at the surface of a cell.

23. The pharmaceutical composition of claim 21, wherein said antibody that binds ILT-2 is capable of enhancing the cytotoxicity of NK cells in a 4-hour in vitro ⁵¹Cr release cytotoxicity assay in which NK cells that express ILT2 are purified from human donors and incubated with target cells that express at their surface HLA-G polypeptides.

24. The pharmaceutical composition of claim 21, wherein said antibody that binds ILT-2 does not bind to any of the wild-type human ILT1, ILT4, ILT5 or ILT6 proteins.

25. The pharmaceutical composition of claim 21, wherein the antibody that binds a human ILT-2 binds: (i) an epitope within the segment of amino acid residues of the ILT2 polypeptide defined by the sequence shown in SEQ ID NO: 110, or (ii) an epitope within the segment of amino acid residues of the ILT2 polypeptide defined by the sequence shown in SEQ ID NO: 111.

26. The pharmaceutical composition of claim 21, wherein the antibody that binds a human ILT-2 has reduced binding to a mutant ILT2 polypeptide comprising the mutations E34A, R36A, Y76I, A82S, R84L with reference to SEQ ID NO: 2, in each case relative to binding between the antibody and a wild-type ILT2 polypeptide comprising the amino acid sequence of SEQ ID NO: 2.

27. The pharmaceutical composition of claim 26, wherein the antibody that binds to a human ILT-2 has furthermore reduced binding to:
(a) a mutant ILT2 polypeptide comprising the mutations G29S, Q30L, Q33A, T32A, D80H with reference to SEQ ID NO: 2;
(b) a mutant ILT2 polypeptide comprising the mutations F299I, Y300R, D301A, W328G, Q378A, K381N with reference to SEQ ID NO: 2;
(c) a mutant ILT2 polypeptide comprising the mutations W328G, Q330H, R347A, T349A, Y350S, Y355A with reference to SEQ ID NO: 2; and/or
(d) a mutant ILT2 polypeptide comprising the mutations D341A, D342S, W344L, R345A, R347A with reference to SEQ ID NO: 2,
in each case relative to binding between the antibody and a wild-type ILT2 polypeptide comprising the amino acid sequence of SEQ ID NO: 2.

28. The pharmaceutical composition of claim 21, wherein the antibody that binds a human ILT-2, optionally in combination with the antibody neutralizes the NKG2A polypeptide, is capable of restoring the cytotoxicity of NK cells toward target cells modified to express at their surface an HLA-G or HLA-A2 polypeptides, wherein said cytotoxicity is restored to at least 60%, 70%, 80% or 90% of the level observed of the NK cells toward parental target cells that do not express said HLA-G or HLA-A2 polypeptides, as determined in a 4-hour in vitro ⁵¹Cr release cytotoxicity assay in which NK cells that express ILT2 are purified from human donors and incubated with target cells.

29. The pharmaceutical composition of claim 21, wherein the antibody that binds a human ILT-2 comprises the heavy and light chains CDR1, 2 and 3 of antibody 2H2A, 48F12, 3F5, 26D8, 18E1 or 27C10.

30. The pharmaceutical composition of claim 21, wherein the antibody that binds a human ILT-2 is antibody 2H2A, 48F12, 3F5, 26D8, 18E1 and 27C10 or a function-conservative variant thereof.

31. The pharmaceutical composition of claim 21, wherein the antibody that binds a human ILT-2 is an antibody having a human Fc domain of the IgG4 isotype or an Fc domain that is modified to reduce binding between the Fc domain and an Fcγ receptor.

32. The pharmaceutical composition of claim 21, wherein the antibody that neutralizes the inhibitory activity of a human NKG2A polypeptide is an antibody that inhibits the binding of HLA-E to NKG2A.

33. The pharmaceutical composition of claim 21, wherein the antibody that neutralizes the inhibitory activity of a human NKG2A polypeptide is monalizumab or a function-conservative variant thereof.

34. A method of treating cancer comprising administering a pharmaceutical composition of claim 21 to an individual having cancer.

35. The method of claim 34, wherein said cancer is a head and neck squamous cell carcinoma (HNSCC), a lung cancer, an NSCLC, a renal cell carcinoma, a colorectal carcinoma, a urothelial cancer or an ovarian cancer.

36. The method of claim 34, further comprising administering an antibody that neutralizes the inhibitory activity of PD-1 to the individual.

37. The method of claim 34, wherein the individual has a tumor characterized by no or low expression of PD-L1 at the tumor cell membrane.

38. A kit for increasing anti-tumor activity toward a tumor of a cancer patient, comprising:
(i) a pharmaceutical composition containing an anti-NKG2A antibody and an antibody that binds ILT-2, or
(ii) a first pharmaceutical composition containing an antibody that binds ILT-2, and a second pharmaceutical composition containing an anti-NKG2A antibody, or
(iii) a pharmaceutical composition containing an anti-NKG2A antibody and instructions to administer said NKG2A neutralizing agent with an antibody that binds ILT-2, or
(iv) a pharmaceutical composition containing an antibody that binds ILT-2 and instructions to administer said antibody that binds ILT-2 with an anti-NKG2A antibody.

39. The kit according to claim 38, wherein said kit further comprises instructions for use in the treatment of a head and neck squamous cell carcinoma (HNSCC), a lung cancer, or an NSCLC.


SEQUENCE LISTING (133 sequences)

SEQ ID NO: 1 - protein, 650 aa, Homo sapiens
MTPILTVLICLGLSLGPRTHVQAGHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 2 - protein, 627 aa, Homo sapiens
GHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 3 - protein, 435 aa, Homo sapiens
GHLPKPTLWAEPGSVIIQGSPVTLRCQGSLQAEEYHLYRENKSASWVRRIQEPGKNGQFPIPSITWEHAGRYHCQYYSHNHSSEYSDPLELVVTGAYSKPTLSALPSPVVTLGGNVTLQCVSQVAFDGFILCKEGEDEHPQRLNSHSHARGWSWAIFSVGPVSPSRRWSYRCYAYDSNSPYVWSLPSDLLELLVPGVSKKPSLSVQPGPMVAPGESLTLQCVSDVGYDRFVLYKEGERDFLQRPGWQPQAGLSQANFTLGPVSPSHGGQYRCYSAHNLSSEWSAPSDPLDILITGQFYDRPSLSVQPVPTVAPGKNVTLLCQSRGQFHTFLLTKEGAGHPPLHLRSEHQAQQNQAEFRMGPVTSAHVGTYRCYSSLSSNPYLLSLPSDPLELVVSASLGQHPQDYTVENLIRMGVAGLVLVVLGILLFEAQHSQR

SEQ ID NO: 4 - protein, 419 aa, Homo sapiens
GPLPKPTLWAEPGSVISWGNSVTIWCQGTLEAREYRLDKEESPAPWDRQNPLEPKNKARFSIPSMTEDYAGRYRCYYRSPVGWSQPSDPLELVMTGAYSKPTLSALPSPLVTSGKSVTLLCQSRSPMDTFLLIKERAAHPLLHLRSEHGAQQHQAEFPMSPVTSVHGGTYRCFSSHGFSHYLLSHPSDPLELIVSGSLEGPRPSPTRSVSTAGPEDQPLMPTGSVPHSGLRRHWEVLIGVLVVSILLLSLLLFLLLQHWRQGKHRTLAQRQADFQRPPGAAEPEPKDGGLQRRSSPAADVQGENFCAAVKNTQPEDGVEMDTRQSPHDEDPQAVTYAKVKHSRPRREMASPPSPLSGEFLDTKDRQAEEDRQMDTEAAASEAPQDVTYAQLHSFTLRQKATEPPPSQEGASPAEPSVYA

SEQ ID NO: 5 - protein, 574 aa, Homo sapiens
GTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 6 - protein, 603 aa, Homo sapiens
GPFPKPTLWAEPGSVISWGSPVTIWCQGSLEAQEYRLDKEGSPEPLDRNNPLEPKNKARFSIPSMTEHHAGRYRCHYYSSAGWSEPSDPLELVMTGFYNKPTLSALPSPVVASGGNMTLRCGSQKGYHHFVLMKEGEHQLPRTLDSQQLHSGGFQALFPVGPVNPSHRWRFTCYYYYMNTPQVWSHPSDPLEILPSGVSRKPSLLTLQGPVLAPGQSLTLQCGSDVGYDRFVLYKEGERDFLQRPGQQPQAGLSQANFTLGPVSPSHGGQYRCYGAHNLSSEWSAPSDPLNILMAGQIYDTVSLSAQPGPTVASGENVTLLCQSWWQFDTFLLTKEGAAHPPLRLRSMYGAHKYQAEFPMSPVTSAHAGTYRCYGSYSSNPHLLSHPSEPLELVVSGHSGGSSLPPTGPPSTPGLGRYLEVLIGVSVAFVLLLFLLLFLLLRRQRHSKHRTSDQRKTDFQRPAGAAETEPKDRGLLRRSSPAADVQEENLYAAVKDTQSEDRVELDSQSPHDEDPQAVTYAPVKHSSPRREMASPPSSLSGEFLDTKDRQVEEDRQMDTEAAASEASQDVTYAQLHSLTLRRKATEPPPSQEGEPPAEPSIYA

SEQ ID NO: 7 - protein, 411 aa, Homo sapiens
GPLPKPTLWAEPGSVITQGSPVTLRCQGSLETQEYHLYREKKTALWITRIPQELVKKGQFPILSITWEHAGRYCCIYGSHTAGLSESSDPLELVVTGAYSKPTLSALPSPVVTSGGNVTIQCDSQVAFDGFILCKEGEDEHPQCLNSHSHARGSSRAIFSVGPVSPSRRWSYRCYGYDSRAPYVWSLPSDLLGLLVPGVSKKPSLSVQPGPVVAPGEKLTFQCGSDAGYDRFVLYKEWGRDFLQRPGRQPQAGLSQANFTLGPVSRSYGGQYTCSGAYNLSSEWSAPSDPLDILITGQIRARPFLSVRPGPTVASGENVTLLCQSQGGMHTFLLTKEGAADSPLRLKSKRQSHKYQAEFPMSPVTSAHAGTYRCYGSLSSNPYLLTHPSDPLELVVSGAAETLSPPQNKSD

SEQ ID NO: 8 - protein, 471 aa, Homo sapiens
ENLPKPILWAEPGPVITWHNPVTIWCQGTLEAQGYRLDKEGNSMSRHILKTLESENKVKLSIPSMMWEHAGRYHCYYQSPAGWSEPSDPLELVVTAYSRPTLSALPSPVVTSGVNVTLRCASRLGLGRFTLIEEGDHRLSWTLNSHQHNHGKFQALFPMGPLTFSNRGTFRCYGYENNTPYVWSEPSDPLQLLVSGVSRKPSLLTLQGPVVTPGENLTLQCGSDVGYIRYTLYKEGADGLPQRPGRQPQAGLSQANFTLSPVSRSYGGQYRCYGAHNVSSEWSAPSDPLDILIAGQISDRPSLSVQPGPTVTSGEKVTLLCQSWDPMFTFLLTKEGAAHPPLRLRSMYGAHKYQAEFPMSPVTSAHAGTYRCYGSRSSNPYLLSHPSEPLELVVSGATETLNPAQKKSDSKTAPHLQDYTVENLIRMGVAGLVLLFLGILLFEAQHSQRSPPRCSQEANSRKDNAPFRVVE

SEQ ID NO: 9 - protein, 437 aa, Homo sapiens
GPFPKPTLWAEPGSVISWGSPVTIWCQGSLEAQEYQLDKEGSPEPLDRNNPLEPKNKARFSIPSMTQHHAGRYRCHYYSSAGWSEPSDPLELVMTGFYNKPTLSALPSPVVASGGNMTLRCGSQKGYHHFVLMKEGEHQLPRTLDSQQLHSGGFQALFPVGPVTPSHRWRFTCYYYYTNTPRVWSHPSDPLEILPSGVSRKPSLLTLQGPVLAPGQSLTLQCGSDVGYDRFVLYKEGERDFLQRPGQQPQAGLSQANFTLGPVSPSHGGQYRCYGAHNLSSEWSAPSDPLNILMAGQIYDTVSLSAQPGPTVASGENVTLLCQSRGYFDTFLLTKEGAAHPPLRLRSMYGAHKYQAEFPMSPVTSAHAGTYRCYGSYSSNPHLLSFPSEPLELMVSASHAKDYTVENLIRMGMAGLVLVFLGILLFEAQHSQRNPQD

SEQ ID NO: 10 - protein, 338 aa, Homo sapiens
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 11 - protein, 358 aa, Homo sapiens
MVDGTLLLLLSEALALTQTWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 12 - protein, 117 aa, mus musculus
QVQLQQSGAELVKPGASVKLSCKASGYTFTEHTIHWIKQRSGQGLEWIGWFYPGSGSMKYNEKFKDKATLTADKSSSTVYMELTRLTSEDSAVYFCARHTNWDFDYWGQGTTLTVSS

SEQ ID NO: 13 - protein, 111 aa, mus musculus
DIVLTQSPASLAVSLGQRATISCKASQSVDYGGDSYMNWYQQKPGQPPKLLIYAASNLESGIPARFSGSGSGTDLTLNIHPVEEDDAAMYYCQQSNEEPWTFGGGTKLEIK

SEQ ID NO: 14 - protein, 5 aa, mus musculus
EHTIH

SEQ ID NO: 15 - protein, 17 aa, mus musculus
WFYPGSGSMKYNEKFKD

SEQ ID NO: 16 - protein, 8 aa, mus musculus
HTNWDFDY

SEQ ID NO: 17 - protein, 15 aa, mus musculus
KASQSVDYGGDSYMN

SEQ ID NO: 18 - protein, 7 aa, mus musculus
AASNLES

SEQ ID NO: 19 - protein, 9 aa, mus musculus
QQSNEEPWT

SEQ ID NO: 20 - protein, 117 aa, mus musculus
QVQLQQSGAELVKPGASVRLSCKASGYTFTAHTIHWVKQRSGQGLEWIGWLYPGSGSIKYNEKFKDKATLTADKSSSTVYMELSRLTSEDSAVYFCARHTNWDFDYWGQGTTLTVSS

SEQ ID NO: 21 - protein, 111 aa, mus musculus
NIVLTQSPASLAVSLGQRATISCKASQSVDYGGASYMNWYQQKPGQPPKLLIYAASNLESGIPARFSGSGSGTDLTLNIHPVEEEDAAMYYCQQSNEEPWTFGGGTKLEIK

SEQ ID NO: 22 - protein, 5 aa, mus musculus
AHTIH

SEQ ID NO: 23 - protein, 17 aa, mus musculus
WLYPGSGSIKYNEKFKD

SEQ ID NO: 24 - protein, 8 aa, mus musculus
HTNWDFDY

SEQ ID NO: 25 - protein, 15 aa, mus musculus
KASQSVDYGGASYMN

SEQ ID NO: 26 - protein, 7 aa, mus musculus
AASNLES

SEQ ID NO: 27 - protein, 9 aa, mus musculus
QQSNEEPWT

SEQ ID NO: 28 - protein, 122 aa, mus musculus
QVQLQQPGAELVKPGASVRMSCKASGYTFTSYWVHWVKQRPGQGLEWIGVIDPSDSYTSYNQNFKGKATLTVDTSSKTAYIHLSSLTSEDSAVYFCARGERYDGDYFAMDYWGQGTSVTVSS

SEQ ID NO: 29 - protein, 107 aa, mus musculus
DIVMTQSPASLSVSVGETVTITCRASENIYSNLAWYQQKQGKSPQLLVYAATNLADGVPSRFSGSRSGTQYSLKINSLQSEDFGTYYCQHFWNTPRTFGGGTKLEIK

SEQ ID NO: 30 - protein, 5 aa, mus musculus
SYWVH

SEQ ID NO: 31 - protein, 17 aa, mus musculus
VIDPSDSYTSYNQNFKG

SEQ ID NO: 32 - protein, 13 aa, mus musculus
GERYDGDYFAMDY

SEQ ID NO: 33 - protein, 11 aa, mus musculus
RASENIYSNLA

SEQ ID NO: 34 - protein, 7 aa, mus musculus
AATNLAD

SEQ ID NO: 35 - protein, 9 aa, mus musculus
QHFWNTPRT

SEQ ID NO: 36 - protein, 118 aa, mus musculus
QVQLKESGPGLVAPSQSLSITCTVSGFSLTSYGVSWVRQPPGKGLEWLGVIWGDGSTNYHSALISRLSISKDNSKSQVFLKLNSLQTDDTATYYCAKPRWDDYAMDYWGQGTSVTVSS

SEQ ID NO: 37 - protein, 106 aa, mus musculus
DIQMTQTTSSLSASLGDRVTISCRASQDISNYLNWYQQKPDGTVKLLIYYTSRLHSGVPSRFSGSGSGTDYSLTISNLEQEDIATYFCQQGNTLWTFGGGTKLEIK

SEQ ID NO: 38 - protein, 120 aa, mus musculus
EVQLQESGPGLVKPSQSLSLTCSVTGYSITSGYYWNWIRQFPENKLEWMGYIRYDGSNNYNPSLNNRISITRDASKNQFFLKLNSVTTEDTATYYCARGWLLWFYAVDYWGQGTSVTVSS

SEQ ID NO: 39 - protein, 112 aa, mus musculus
DVVMTQTPLSLPVSLGDQASISCRSSQSIVHTNGNTYLEWYLQKSGQSPKLLIYKVSNRLSGVPDRFSGSGSGTDFTLKISRVEAEDLGIYYCFQGSHVPWTFGGGTKLEIK

SEQ ID NO: 40 - protein, 117 aa, mus musculus
QVQLKESGPGLVAPSQSLSITCTVSGFSLTSYGVSWVRQPPGKGLEWLGVIWGDGNTNYHSALISRLSISKDNSKSQVFLKLNSLQTDDTATYYCARTNWDGWFAYWGQGTLVTVSA

SEQ ID NO: 41 - protein, 108 aa, mus musculus
DIVMTQSHKFMSTSVGDRVSITCKASQDVGTAVAWYQQKPGQSPKLLIYWASTRHTGVPDRFTGSGSGTDFTLTISNVQSEDLADYFCQQYRSYPLGTFGGGTKLEIK

SEQ ID NO: 42 - protein, 119 aa, mus musculus
DVQLQESGPELVKPGASVKISCKSSGYSFTNFYIHWVKQRPGQGLDWIGWIFPGTGETNFNEKFKVKAALTADTSSSTVYMQLSTLTSEDSAVYFCARSWNYDARWGYWGQGTSITVSS

SEQ ID NO: 43 - protein, 107 aa, mus musculus
DIQMTQTTSSLSASLGDRVTISCRASQDISNYLNWYQQKVDGTVKLLISYTSRLHSGVPSRFSGSGSGTDYSLTISNLEQEDIATYFCQQGITLPLTFGAGTKLELK

SEQ ID NO: 44 - protein, 119 aa, mus musculus
QVQLKESGPELVKPGASVKISCKASGYSFTNYYIHWVKQRPGQGLEWIGWIFPGSGETNYSEKFKGEAILTADTSSNTAYMQLSSLTSEDSAVYFCARSWNYDARWGYWGQGTTLTVSS

SEQ ID NO: 45 - protein, 111 aa, mus musculus
EIVLTQSPASLAVSLGQRATISCRASEIIDSYGISFMHWYQQKPGQPPKLLIYRASNLESGIPARFSGSGSRTDFTLTINPVEADDVATYYCQQSNEDPFTFGSGTKLEIK

SEQ ID NO: 46 - protein, 119 aa, mus musculus
EVKLEESGPELVKPGASVKLSCKASGYTFTNYYMQWVKQRPGQGLEWIGWIFPGSGESSYNEKFKGKATLSADTSSTTAYMQLSSLTSEDSAVYFCARTWNYDARWGYWGQGTTLTVSS

SEQ ID NO: 47 - protein, 111 aa, mus musculus
DILMTQSPASLAVSLGQRATISCIPSESIDSYGISFMHWYQQKPGQPPKLLIYRASNLESGIPARFSGSGSRTDFTLTINPVEADDVATYYCQQSNEDPFTFGSGTKLELK

SEQ ID NO: 48 - protein, 5 aa, mus musculus
NYYIQ

SEQ ID NO: 49 - protein, 17 aa, mus musculus
WIFPGNNETNYNEKFKG

SEQ ID NO: 50 - protein, 10 aa, mus musculus
SWNYDARWGY

SEQ ID NO: 51 - protein, 15 aa, mus musculus
RASEIIDSYGISFMH

SEQ ID NO: 52 - protein, 7 aa, mus musculus
RASNLES

SEQ ID NO: 53 - protein, 9 aa, mus musculus
QQSNEDPFT

SEQ ID NO: 54 - protein, 5 aa, mus musculus
SYGVS

SEQ ID NO: 55 - protein, 16 aa, mus musculus
IIWGDGSTNYHSALVS

SEQ ID NO: 56 - protein, 10 aa, mus musculus
PNWDYYAMDY

SEQ ID NO: 57 - protein, 11 aa, mus musculus
RASQDISNYLN

SEQ ID NO: 58 - protein, 7 aa, mus musculus
YTSRLHS

SEQ ID NO: 59 - protein, 9 aa, mus musculus
QQGITLPLT

SEQ ID NO: 60 - protein, 5 aa, mus musculus
NYYMQ

SEQ ID NO: 61 - protein, 17 aa, mus musculus
WIFPGSGESNYNEKFKG

SEQ ID NO: 62 - protein, 17 aa, mus musculus
WIFPGSGESSYNEKFKG

SEQ ID NO: 63 - protein, 10 aa, mus musculus
TWNYDARWGY

SEQ ID NO: 64 - protein, 15 aa, mus musculus
IPSESIDSYGISFMH

SEQ ID NO: 65 - protein, 7 aa, mus musculus
RASNLES

SEQ ID NO: 66 - protein, 9 aa, mus musculus
QQSNEDPFT

SEQ ID NO: 67 - protein, 233 aa, homo sapiens
MDNQGVIYSDLNLPPNPKRQQRKPKGNKSSILATEQEITYAELNLQKASQDFQGNDKTYHCKDLPSAPEKLIVGILGIICLILMASVVTIVVIPSTLIQRHNNSSLNTRTQKARHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

SEQ ID NO: 68 - protein, 124 aa, Artificial
  Synthetic
EVQLVQSGAEVKKPGESLKISCKGSGYSFTSYWMNWVRQMPGKGLEWMGRIDPYDSETHYSPSFQGQVTISADKSISTAYLQWSSLKASDTAMYYCARGGYDFDVGTLYWFFDVWGQGTTVTVS

SEQ ID NO: 69 - protein, 124 aa, Artificial
  Synthetic
QVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVS

SEQ ID NO: 70 - protein, 124 aa, artificial
  synthetic
EVQLVQSGAEVKKPGESLRISCKGSGYSFTSYWMNWVRQMPGKGLEWMGRIDPYDSETHYSPSFQGHVTISADKSISTAYLQWSSLKASDTAMYYCARGGYDFDVGTLYWFFDVWGQGTTVTVS

SEQ ID NO: 71 - protein, 124 aa, artificial
  synthetic
EVQLVQSGAEVKKPGATVKISCKVSGYTFTSYWMNWVQQAPGKGLEWMGRIDPYDSETHYAEKFQGRVTITADTSTDTAYMELSSLRSEDTAVYYCATGGYDFDVGTLYWFFDVWGQGTTVTVS

SEQ ID NO: 72 - protein, 124 aa, artificial
  synthetic
QVQLVQSGAEVKKPGASVKVSCKASGYTFTSYWMNWVRQAPGQGLEWMGRIDPYDSETHYAQKFQGRVTMTRDTSTSTVYMELSSLRSEDTAVYYCARGGYDFDVGTLYWFFDVWGQGTTVTVS

SEQ ID NO: 73 - protein, 107 aa, artificial
  synthetic
DIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIK

SEQ ID NO: 74 - protein, 452 aa, artificial
  synthetic
EVQLVQSGAEVKKPGESLKISCKGSGYSFTSYWMNWVRQMPGKGLEWMGRIDPYDSETHYSPSFQGQVTISADKSISTAYLQWSSLKASDTAMYYCARGGYDFDVGTLYWFFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 75 - protein, 452 aa, artificial
  synthetic
QVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 76 - protein, 452 aa, artificial
  synthetic
EVQLVQSGAEVKKPGESLRISCKGSGYSFTSYWMNWVRQMPGKGLEWMGRIDPYDSETHYSPSFQGHVTISADKSISTAYLQWSSLKASDTAMYYCARGGYDFDVGTLYWFFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 77 - protein, 452 aa, artificial
  synthetic
EVQLVQSGAEVKKPGATVKISCKVSGYTFTSYWMNWVQQAPGKGLEWMGRIDPYDSETHYAEKFQGRVTITADTSTDTAYMELSSLRSEDTAVYYCATGGYDFDVGTLYWFFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 78 - protein, 452 aa, artificial
  synthetic
QVQLVQSGAEVKKPGASVKVSCKASGYTFTSYWMNWVRQAPGQGLEWMGRIDPYDSETHYAQKFQGRVTMTRDTSTSTVYMELSSLRSEDTAVYYCARGGYDFDVGTLYWFFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 79 - protein, 214 aa, artificial
  synthetic
DIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 5 aa, mus musculus
SYWMN

SEQ ID NO: 81 - protein, 17 aa, artificial
  synthetic
RIDPYDSETHYAQKLQG

SEQ ID NO: 82 - protein, 16 aa, mus musculus
GGYDFDVGTLYWFFDV

SEQ ID NO: 83 - protein, 11 aa, mus musculus
RASENIYSYLA

SEQ ID NO: 84 - protein, 7 aa, mus musculus
NAKTLAE

SEQ ID NO: 85 - protein, 9 aa, mus musculus
QHHYGTPRT

SEQ ID NO: 86 - protein, 5 aa, mus musculus
SHSMN

SEQ ID NO: 87 - protein, 17 aa, mus musculus
AISSSSSYIYYADSVKG

SEQ ID NO: 88 - protein, 9 aa, mus musculus
EEWGLPFDY

SEQ ID NO: 89 - protein, 11 aa, mus musculus
RASQGISSALA

SEQ ID NO: 90 - protein, 11 aa, mus musculus
RASQGIPSALA

SEQ ID NO: 91 - protein, 11 aa, mus musculus
RASQGINSALA

SEQ ID NO: 92 - protein, 7 aa, mus musculus
DASSLKS

SEQ ID NO: 93 - protein, 9 aa, mus musculus
QQFNSYPLT

SEQ ID NO: 94 - protein, 5 aa, mus musculus
NTYIH

SEQ ID NO: 95 - protein, 16 aa, mus musculus
IDPANADTKYAPTFQG

SEQ ID NO: 96 - protein, 12 aa, mus musculus
YRDYLFYYALGY

SEQ ID NO: 97 - protein, 16 aa, mus musculus
RSSKSLLHSNANTYLY

SEQ ID NO: 98 - protein, 7 aa, mus musculus
RMSNLAS

SEQ ID NO: 99 - protein, 9 aa, mus musculus
MQHLEYPYT

SEQ ID NO: 100 - protein, 16 aa, mus musculus
IDPANGDTKYAPTFQG

SEQ ID NO: 101 - protein, 16 aa, mus musculus
RSSKSLLHSNGNTYLY

SEQ ID NO: 102 - protein, 16 aa, mus musculus
IDPANGDTKYAPKFQG

SEQ ID NO: 103 - protein, 12 aa, mus musculus
YGNYLYYYSLDY

SEQ ID NO: 104 - protein, 5 aa, mus musculus
NTYMH

SEQ ID NO: 105 - protein, 16 aa, mus musculus
IDPADGDTQYAPKFQG

SEQ ID NO: 106 - protein, 12 aa, mus musculus
YGNYLFYYSMDY

SEQ ID NO: 107 - protein, 16 aa, mus musculus
IDPANGDTQYDPKFQG

SEQ ID NO: 108 - protein, 12 aa, mus musculus
YGDYLFYYSLKY

SEQ ID NO: 109 - protein, 9 aa, mus musculus
MQHLESPYT

SEQ ID NO: 110 - protein, 119 aa, mus musculus
EVQLVESGGGLVKPGGSLKLSCAASGFTFSSYAMSWVRQSPEKRLEWVAEISSGGSYTYYPDTVTGRFTISRDNAKNTLYLEISSLRSEDTAMYYCTRHGDYPRFFDVWGAGTTVTVSS

SEQ ID NO: 111 - protein, 106 aa, mus musculus
QIVLTQSPALMSASPGEKVTMTCSASSSVSYIYWYQQKPRSSPKPWIYLTSNLASGVPARFSGSGSGTSYSLTISSMEAEDAATYYCQQWSGNPYTFGGGTKLEIK

SEQ ID NO: 112 - protein, 138 aa, artificial
  synthetic
TGVHSGKPIPNPLLGLDSTGHLPKPTLWAEPGSVITQGSPVTLRCQGGQETQEYRLYREKKTALWITRIPQELVKKGQFPIPSITWEHAGRYRCYYGSDTAGRSESSDPLELVVTGAGALQSTASLFVVSLSLLHLYS

SEQ ID NO: 113 - protein, 141 aa, artificial
  synthetic
TGVHSGKPIPNPLLGLDSTYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVGALQSTASLFVVSLSLLHLYS

SEQ ID NO: 114 - protein, 139 aa, artificial
  synthetic
TGVHSGKPIPNPLLGLDSTSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQGALQSTASLFVVSLSLLHLYS

SEQ ID NO: 115 - protein, 348 aa, artificial
  synthetic
TGVHSGKPIPNPLLGLDSTFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 116 - protein, 239 aa, artificial
  synthetic
TGVHSGKPIPNPLLGLDSTGHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVGALQSTASLFVVSLSLLHLYS

SEQ ID NO: 117 - protein, 240 aa, artificial
  synthetic
TGVHSGKPIPNPLLGLDSTYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQGALQSTASLFVVSLSLLHLYS

SEQ ID NO: 118 - protein, 447 aa, artificial
  synthetic
TGVHSGKPIPNPLLGLDSTSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 119 - protein, 338 aa, artificial
  synthetic
TGVHSGKPIPNPLLGLDSTGHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQGALQSTASLFVVSLSLLHLYS

SEQ ID NO: 120 - protein, 548 aa, artificial
  synthetic
TGVHSGKPIPNPLLGLDSTYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 121 - protein, 98 aa, homo sapiens
GHLPKPTLWAEPGSVITQGSPVTLRCQGGQETQEYRLYREKKTALWITRIPQELVKKGQFPIPSITWEHAGRYRCYYGSDTAGRSESSDPLELVVTGA

SEQ ID NO: 122 - protein, 137 aa, homo sapiens
FYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRH

SEQ ID NO: 123 - protein, 330 aa, artificial
  synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - protein, 330 aa, artificial
  synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - protein, 330 aa, artificial
  synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 330 aa, artificial
  synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - DNA, 35 bp, artificial
  synthetic
acaggcgtgcattcggggcacctccccaagcccac

SEQ ID NO: 128 - DNA, 58 bp, artificial
  synthetic
cgaggtcgggggatcctcaatggtggtgatgatggtggtgccttcccagaccactctg

SEQ ID NO: 129 - protein, 441 aa, artificial
  synthetic
GHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHHHHHHH

SEQ ID NO: 130 - DNA, 33 bp, artificial
  synthetic
acaggcgtgcattcggggcacctccccaagccc

SEQ ID NO: 131 - protein, 36 aa, artificial
  synthetic
CCGCCCCGACTCTAGACTAGTGGATGGCCAGAGTGG

SEQ ID NO: 132 - DNA, 43 bp, artificial
  synthetic
ccagaacacaggatccgccgccaccatggtggtcatggcgccc

SEQ ID NO: 133 - DNA, 36 bp, artificial
  synthetic
ttttctaggtctcgagtcaatctgagctcttctttc